(19) United States
(12) Patent Application Publication
(10) Pub. No.: US 2022/0211703 A1
(43) Pub. Date: Jul. 7, 2022

(54) METHOD FOR TREATING COUGH BY USING DIAMINOPYRIMIDINE COMPOUND

(71) Applicant: BEIJING TIDE PHARMACEUTICAL CO., LTD., Beijing (CN)

(72) Inventors: Yanping ZHAO, Beijing (CN); Hongjun WANG, Beijing (CN); Huai HUANG, Beijing (CN); Yuanyuan JIANG, Beijing (CN); Huining LIANG, Beijing (CN); Ran AN, Beijing (CN); Zhou LAN, Beijing (CN); Jin WANG, Beijing (CN); Liying ZHOU, Beijing (CN); Yanan LIU, Beijing (CN)

(21) Appl. No.: 17/607,438

(22) PCT Filed: Apr. 29, 2020
(86) PCT No.: PCT/CN2020/087686

§ 371 Date: Oct. 29, 2021

(30) Foreign Application Priority Data
Apr. 30, 2019 (CN) PCT/CN2019/085180

(51) Int. Cl. A61K 31/506; A61P 11/14

(52) U.S. Cl. CPC A61K 31/506; A61P 11/14

(57) ABSTRACT

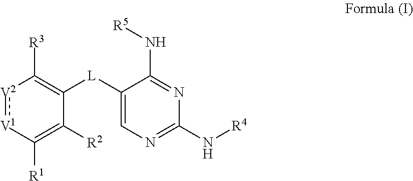

A method for treating, suppressing or alleviating cough or cough impulse in the field of biomedicine, comprising: administering to a subject in need thereof a therapeutically effective amount of a diaminopyrimidine compound of formula (I) or a pharmaceutically acceptable salt, ester, stereoisomer, polymorph, solvate, N-oxide, isotope-labeled compound, metabolite or prodrug thereof.

### FIELD OF THE INVENTION

The present invention belongs to the field of biomedicine, and specifically relates to a method for treating, suppressing or alleviating cough or cough impulse, comprising administering to a subject in need thereof a therapeutically effective amount of a diaminopyrimidine compound or a pharmaceutically acceptable salt, ester, stereoisomer, polymorph, solvate, N-oxide, isotope-labeled compound, metabolite or prodrug thereof.

### BACKGROUND OF THE INVENTION

Cough is a common respiratory symptom, which is caused by inflammation, foreign body, physical or chemical stimuli to tracheal and bronchial mucosae or pleurae. It manifests firstly as closure of glottis, contraction of respiratory muscles, and increase of intrapulmonic pressure, and then as opening of glottis, and air injection from the lung, usually accompanied with sound. A cough plays a protective role in clearing airway foreign bodies and secretions. If cough persists and turns from acute to chronic, it may bring a patient greater pain, such as chest distress, throat itching and/or gasping, etc.

The cause and recurrent attack of cough usually result from combined effects of various complicated factors. The factors causing cough typically include inhaled material, infection, food, climate change, etc. There still lack effective drugs for treating cough caused by various factors at present.

### SUMMARY OF THE INVENTION

In one aspect, the present invention provides a method for treating, suppressing or alleviating cough or cough impulse, comprising administering to a subject in need thereof a therapeutically effective amount of a compound of Formula (I) or a pharmaceutically acceptable salt, ester, stereoisomer, polymorph, solvate, N-oxide, isotopically labeled compound, metabolite or prodrug thereof:

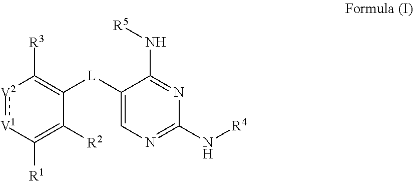

wherein:

L is selected from the group consisting of C(═O), CRR′, NR, O, S, S═O and S(═O)₂;

V¹ is selected from the group consisting of N,

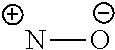

### and NR;

V² is selected from the group consisting of CR⁶ and C(═O); [symbol] represents either a single bond or a double bond, provided that when [symbol] is a single bond, V¹ is NR and V² is C(═O);

R and R′ are each independently selected from the group consisting of H, halogen, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);

R¹, R², R³ and R⁶ are each independently selected from the group consisting of H, halogen, —CN, —NO₂, —NH₂, —OH, —SH, —Se—R, —Si(R)₃, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl, C₆₋₁₂ aralkyl, C₁₋₆ haloalkyl, —C(═O)R^{a}, —OC(═O)R^{a}, —C(═O)R^{a}, —OR^{a}, —SR^{a}, —S(═O)R^{a}, —S(═O)₂R^{a}, —S(═O)₂NR^{a}R^{b}, —S(═O)(═NR)R^{a}, —NR^{a}R^{b}, —C(═O)NR^{a}R^{b}, —C(═S)NR^{a}R^{b}, —C(═NR)NR^{a}R^{b}, —NR^{a}—C(═O)R^{b}, —NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-OR^{a}, —C₁₋₆ alkylene-C(═O)R, —C₁₋₆ alkenylene-OR^{a}, —O—C₁₋₆ alkylene-NR^{a}R^{b} and —P(═O)R^{a}R^{b};

R⁴ and R⁵ are each independently selected from the group consisting of H, —C(═O)OR^{a}, —NR^{a}R^{b}, —NR^{a}—C(═O)R^{b}, —NR^{a}—C(═O)OR^{b}, —C₁₋₆ alkylene-NR^{a}R^{b}, —C₁₋₆ alkylene-OR^{a}, —C₁₋₆ alkylene-O—C₁₋₆ alkylene-OR^{a}, C₁₋₆ alkyl, C₁₋₆ haloalkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl;
alternatively, R¹ and R⁴ together form —NH—(C₁₋₆ alkylene)-L-(C₁₋₆ alkylene)-, preferably —NHCH₂CH₂—O—CH₂CH₂—;
the above alkyl, alkylene, alkenyl, alkynyl, cyclic hydrocarbyl, heterocyclyl, aryl, heteroaryl and aralkyl, at each occurrence, are each optionally substituted with one or more substituents independently selected from the group consisting of halogen, hydroxyl, oxo, amino, cyano, nitro, —Si(R)₃, C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl, C₆₋₁₂ aralkyl, —C(═O)R^{a}, —OC(═O)R^{a}, —C(═O)OR^{a}, —OR^{a}, —SR^{a}, —S(═O)R^{a}, —S(═O)₂R^{a}, —S(═O)₂NR^{a}R^{b}, —NR^{a}R^{b}, —C(═O)NR^{a}R^{b}, —NR^{a}—C(═O)R^{b}, —NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-OR^{a}, —C₁₋₆ alkenylene-OR^{a} and —O—C₁₋₆ alkylene-NR^{a}R^{b}, the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, —NR^{a}R^{b}, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl; and

R^{a} and R^{b}, at each occurrence, are each independently selected from the group consisting of H, —OH, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl; alternatively, R^{a} and R^{b} together with the atom to which they are attached form a 3- to 12-membered heterocycle or heteroaromatic ring, the above groups are further optionally substituted with one or more substituents independently selected from the group consisting of halogen, hydroxyl, oxo, amino, cyano, nitro, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl.

In another aspect, the present invention provides use of a compound of above Formula (I) or a pharmaceutically acceptable salt, ester, stereoisomer, polymorph, solvate, N-oxide, isotopically labeled compound, metabolite or prodrug thereof in the manufacture of a medicament for treating, suppressing or alleviating cough or cough impulse.

In another aspect, the present invention provides a compound of above Formula (I) or a pharmaceutically acceptable salt, ester, stereoisomer, polymorph, solvate, N-oxide, isotopically labeled compound, metabolite or prodrug thereof for use of treating, suppressing or alleviating cough or cough impulse.

### BRIEF DESCRIPTION OF THE DRAWINGS

FIG. 1 shows the antitussive effect of Compound 2 at doses of 23 mg/kg and 92 mg/kg.

FIG. 2 shows the antitussive effect of compound 66 at doses of 46 mg/kg and 92 mg/kg.

FIG. 3 shows the antitussive effect of compound 66 at doses of 5.1 mg/kg, 15.3 mg/kg and 46 mg/kg.

FIG. 4 shows the effect of compound 66 on the P2X3-mediated current in 1321N1 cells stably transfected with P2X3.

FIG. 5 shows the concentration-inhibition curve of compound 66 on the P2X3-mediated current in 1321N1 cells stably transfected with P2X3.

FIG. 6 shows the effect of compound 66 on the P2X3-mediated current in rat dorsal root ganglion cells.

FIG. 7 shows the concentration-inhibition curve of compound 66 on the P2X3-mediated current in rat dorsal root ganglion cells.

### DETAILED DESCRIPTION OF THE INVENTION

### Definition

Unless otherwise defined in the context, all technical and scientific terms used herein are intended to have the same meaning as commonly understood by a person skilled in the art. References to techniques employed herein are intended to refer to the techniques as commonly understood in the art, including variations on those techniques or substitutions of equivalent techniques which would be apparent to a person skilled in the art. While it is believed that the following terms will be readily understood by a person skilled in the art, the following definitions are nevertheless put forth to better illustrate the present invention.

The terms “contain”, “include”, “comprise”, “have”, or “relate to”, as well as other variations used herein are inclusive or open-ended, and do not exclude additional, unrecited elements or method steps.

As used herein, the term “alkylene” refers to a saturated divalent hydrocarbyl, preferably refers to a saturated divalent hydrocarbyl having 1, 2, 3, 4, 5 or 6 carbon atoms, e.g., methylene, ethylene, propylene or butylene.

As used herein, the term “alkyl” is defined as a linear or branched saturated aliphatic hydrocarbon. In some embodiments, alkyl has 1-12, e.g., 1-6, carbon atoms. For example, as used herein, the term “C₁₋₆ alkyl” refers to a linear or branched group having 1-6 carbon atoms (such as methyl, ethyl, n-propyl, isopropyl, n-butyl, isobutyl, sec-butyl, tent-butyl, n-pentyl, isopentyl, neopentyl, or n-hexyl), which is optionally substituted with one or more (e.g., 1 to 3) suitable substituents such as halogen (in which case the group may be referred to as “haloalkyl”) (e.g., CH₂F, CHF₂, CF₃, CCl₃, C₂F₅, C₂Cl₅, CH₂CF₃, CH₂Cl or —CH₂CH₂CF₃ etc.). The term “C₁₋₄ alkyl” refers to a linear or branched aliphatic hydrocarbon chain having 1-4 carbon atoms (i.e., methyl, ethyl, n-propyl, isopropyl, n-butyl, isobutyl, sec-butyl or tent-butyl).

As used herein, the term “alkenyl” refers to a linear or branched monovalent hydrocarbyl having a double bond and 2-6 carbon atoms (“C₂₋₆ alkenyl”). The alkenyl is e.g., vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl and 4-methyl-3-pentenyl. When the compound of the present invention contains an alkenylene group, the compound may exist as the pure E (entgegen) form, the pure Z (zusammen) form, or any mixture thereof.

As used herein, the term “alkynyl” refers to a monovalent hydrocarbyl containing one or more triple bond, and preferably having 2, 3, 4, 5 or 6 carbon atoms, e.g., ethynyl or propynyl.

As used herein, the term “cycloalkyl” refers to a saturated monocyclic or polycyclic (e.g., bicyclic) hydrocarbon ring (e.g., monocyclic, such as cyclopropyl, cyclobutyl, cyclopentyl, cyclohexyl, cycloheptyl, cyclooctyl, or cyclononyl, or bicyclic, including spiro, fused or bridged cyclic system (such as bicyclo[1.1.1]pentyl, bicyclo[2.2.1]heptyl, bicyclo[3.2.1]octyl or bicyclo[5.2.0]nonyl, or decahydronaphthalene etc.)), which is optionally substituted with one or more (e.g., 1 to 3) suitable substituents. The cycloalkyl has 3 to 15 carbon atoms. For example, the term “C₃₋₆ cycloalkyl” refers to a saturated monocyclic or polycyclic (e.g., bicyclic) hydrocarbon ring having 3 to 6 ring forming carbon atoms (e.g., cyclopropyl, cyclobutyl, cyclopentyl, or cyclohexyl), which is optionally substituted with one or more (e.g., 1 to 3) suitable substituents, e.g., methyl substituted cyclopropyl.

As used herein, the terms “cyclic hydrocarbylene”, “cyclic hydrocarbyl” and “hydrocarbon ring” refer to a saturated (i.e., “cycloalkylene” and “cycloalkyl”) or unsaturated (i.e., having one or more double and/or triple bonds in the ring) monocyclic or polycyclic hydrocarbon ring having e.g., 3-10 (suitably having 3-8, and more suitably having 3-6) ring carbon atoms, including but not limited to cyclopropyl(ene) (ring), cyclobutyl(ene) (ring), cyclopentyl(ene) (ring), cyclohexyl(ene) (ring), cycloheptyl(ene) (ring), cyclooctyl(ene) (ring), cyclononyl(ene) (ring), cyclohexenyl(ene) (ring), and the like.

As used herein, the terms “heterocyclyl”, “heterocyclylene” and “heterocycle” refer to a saturated (i.e., heterocycloalkyl) or partially unsaturated (i.e., having one or more double and/or triple bonds in the ring) cyclic group having e.g., 3-10 (suitably having 3-8, and more suitably having 3-6) ring atoms, wherein at least one ring atom is a heteroatom selected from the group consisting of N, O and S, and the remaining ring atoms are C. For example, “3- to 10-membered heterocyclyl(ene)” of “3- to 10-membered heterocycle” refers to saturated or partially unsaturated heterocyclyl(ene) or heterocycle having 2-9 (e.g., 2, 3, 4, 5, 6, 7, 8 or 9) ring carbon atoms and one or more (e.g., 1, 2, 3, or 4) heteroatoms independently selected from the group consisting of N, O and S. Examples of heterocyclylene, heterocyclyl and heterocycle include, but are not limited to oxiranyl(ene), aziridinyl(ene), azetidinyl(ene), oxetanyl(ene), tetrahydrofuranyl(ene), dioxolinyl(ene), pyrrolidinyl(ene), pyrrolidonyl(ene), imidazolidinyl(ene), pyrazolidinyl(ene), pyrrolinyl(ene), tetrahydropyranyl(ene), piperidinyl(ene), morpholinyl(ene), dithianyl(ene), thiomorpholinyl(ene), piperazinyl(ene) or trithianyl(ene). Said group also encompasses a bicyclic system, including a spiro, fused, or bridged system (e.g., 8-azaspiro[4.5]decane, 3,9-diazaspiro[5.5]undecane, 2-azabicyclo[2.2.2]octane, etc.). Heterocyclylene, heterocyclyl and heterocycle may optionally be substituted with one or more (e.g., 1, 2, 3 or 4) suitable substituents. As used herein, the terms “aryl(ene)” and “aromatic ring” refer to an all-carbon monocyclic or fused-ring polycyclic aromatic group having a conjugated 7E electron system. For example, as used herein, the terms “C₆₋₁₀ aryl(ene)” and “C₆₋₁₀ aromatic ring” refer to an aromatic group containing 6 to 10 carbon atoms, such as phenyl(ene) (benzene ring) or naphthyl(ene) (naphthalene ring). Aryl(ene) or aromatic ring is optionally substituted with one or more (such as 1 to 3) suitable substituents (e.g., halogen, -OH, -CN, —NO₂, and C₁₋₆ alkyl, etc.).

As used herein, the terms “heteroaryl(ene)” and “heteroaromatic ring” refer to a monocyclic, bicyclic or tricyclic aromatic ring system having 5, 6, 8, 9, 10, 11, 12, 13 or 14 ring atoms, particularly 1 or 2 or 3 or 4 or 5 or 6 or 9 or 10 carbon atoms, and containing at least one heteroatom (such as O, N, or S), which can be same to different. Moreover, in each case, it can be benzo-fused. In particular, “heteroaryl(ene)” or “heteroaromatic ring” is selected from the group consisting of thienyl(ene), furyl(ene), pyrrolyl(ene), oxazolyl(ene), thiazolyl(ene), imidazolyl(ene), pyrazolyl(ene), isoxazolyl(ene), isothiazolyl(ene), oxadiazolyl(ene), triazolyl(ene), thiadiazolyl(ene) etc., and benzo derivatives thereof; or pyridinyl(ene), pyridazinyl(ene), pyrimidinyl(ene), pyrazinyl(ene), triazinyl(ene), etc., and benzo derivatives thereof.

As used herein, the term “aralkyl” preferably means aryl or heteroaryl substituted alkyl, wherein aryl, heteroaryl and alkyl are as defined herein. Normally, the aryl group may have 6-14 carbon atoms, the heteroaryl group may have 5-14 ring atoms, and the alkyl group may have 1-6 carbon atoms. Exemplary aralkyl group includes, but is not limited to, benzyl, phenylethyl, phenylpropyl, phenylbutyl.

As used herein, the term “halo” or “halogen” are defined to include F, Cl, Br, or I.

As used herein, the term “nitrogen containing heterocycle” refers to a saturated or unsaturated monocyclic or bicyclic group having 2, 3, 4, 5, 6, 7, 8, 9, 10, 11, 12 or 13 carbon atoms and at least one nitrogen atom in the ring, which may further optionally comprise one or more (e.g., one, two, three or four) ring members selected from the group consisting of N, O, C═O, S, S═O and S(═O)₂. The nitrogen containing heterocycle is attached to the rest of the molecule through the nitrogen atom and any other ring atom in said nitrogen containing heterocycle. The nitrogen containing heterocycle is optionally benzo-fused, and is preferably attached to the rest of the molecule through the nitrogen atom in said nitrogen containing heterocycle and any carbon atom in the fused benzene ring.

The term “substituted” means that one or more (e.g., one, two, three, or four) hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

If a substituent is described as being “optionally substituted”, the substituent may be either (1) not substituted, or (2) substituted. If a carbon of a substituent is described as being optionally substituted with one or more of a list of substituents, one or more of the hydrogens on the carbon (to the extent there are any) may separately and/or together be replaced with an independently selected optional substituent. If a nitrogen of a substituent is described as being optionally substituted with one or more from a list of substituents, one or more of the hydrogens on the nitrogen (to the extent there are any) may each be replaced with an independently selected optional substituent.

If substituents are described as being “independently selected” from a group, each substituent is selected independent of the other(s). Each substituent therefore may be identical to or different from the other substituent(s).

As used herein, the term “one or more” means one or more than one (e.g., 2, 3, 4, 5 or 10) as reasonable.

As used herein, unless specified, the point of attachment of a substituent can be from any suitable position of the substituent.

When a bond to a substituent is shown to cross a bond connecting two atoms in a ring, then such substituent may be bonded to any of the ring-forming atoms in that ring that are substitutable.

The present invention also includes all pharmaceutically acceptable isotopically labeled compounds, which are identical to those of the present invention except that one or more atoms are replaced by an atom having the same atomic number, but an atomic mass or mass number different from the atomic mass or mass number which predominates in nature. Examples of isotopes suitable for inclusion in the compound of the present invention include, but are not limited to, isotopes of hydrogen, such as ²H, ³H; carbon, such as ¹¹C, ¹³C, and ¹⁴C; chlorine, such as ³⁶Cl; fluorine, such as ¹⁸F; iodine, such as ¹²³I and ¹²⁵I; nitrogen, such as ¹³N and ¹⁵N; oxygen, such as ¹⁵O, ¹⁷O, and ¹⁸O; phosphorus, such as ³²P; and sulfur, such as ³⁵S. Certain isotopically labeled compounds of the present invention, for example those incorporating a radioactive isotope, are useful in drug and/or substrate tissue distribution studies (e.g., assays). The radioactive isotopes tritium, i.e., ³H, and carbon-14, i.e., ¹⁴C, are particularly useful for this purpose in view of their ease of incorporation and ready means of detection. Substitution with positron-emitting isotopes, such as ¹¹C, ¹⁸F, ¹⁵O and ¹³N, can be useful in positron emission tomography (PET) studies for examining substrate receptor occupancy. Isotopically labeled compounds of the present invention can generally be prepared by processes analogous to those described in the accompanying Schemes and/or in the Examples and Preparations, by using an appropriate isotopically labeled reagent in place of the non-labeled reagent previously employed. Pharmaceutically acceptable solvates in accordance with the invention include those wherein the solvent of crystallization may be isotopically substituted, e.g., D₂O, acetone-d₆, or DMSO-d₆.

The term “stereoisomer” refers to isomers with at least one asymmetric center. A compound having one or more (e.g., one, two, three or four) asymmetric centers can give rise to a racemic mixture, single enantiomer, diastereomer mixture and individual diastereomer. Certain individual molecules may exist as geometric isomers (cis/trans). Similarly, the compound of the present invention may exist as a mixture of two or more structurally different forms in rapid equilibrium (generally referred to as tautomer). Typical examples of a tautomer include a keto-enol tautomer, phenol-keto tautomer, nitroso-oxime tautomer, imine-enamine tautomer and the like. It is to be understood that all such isomers and mixtures thereof in any proportion (such as 60%, 65%, 70%, 75%, 80%, 85%, 90%, 95%, 96%, 97%, 98%, and 99%) are encompassed within the scope of the present invention.

The chemical bonds of the compound of the present invention may be depicted herein using a solid line ( [symbol] ), a solid wedge ( [symbol] ), or a dotted wedge ( [symbol] ). The use of a solid line to depict bonds to asymmetric carbon atoms is meant to indicate that all possible stereoisomers (e.g., specific enantiomers, racemic mixtures, etc.) at that carbon atom are included. The use of either a solid or dotted wedge to depict bonds to asymmetric carbon atoms is meant to indicate that the stereoisomer shown is present. When present in racemic compounds, solid and dotted wedges are used to define relative stereochemistry, rather than absolute stereochemistry. Unless stated otherwise, it is intended that the compound of the present invention can exist as stereoisomers, which include cis and trans isomers, optical isomers such as R and S enantiomers, diastereomers, geometric isomers, rotational isomers, conformational isomers, atropisomers, and mixtures thereof. The compound of the present invention may exhibit more than one type of isomerism, and consist of mixtures thereof (such as racemates and diastereomeric pairs).

The present invention includes all possible crystalline forms or polymorphs of the compound of the present invention, either as a single polymorph, or as a mixture of more than one polymorphs, in any ratio.

It also should be understood that, certain compounds of the present invention can be used for the treatment in a free form, or where appropriate, in a form of a pharmaceutically acceptable derivative. In the present invention, the pharmaceutically acceptable derivative includes, but is not limited to a pharmaceutically acceptable salt, ester, solvate, N-oxide, metabolite or prodrug, which can directly or indirectly provide the compound of the present invention or a metabolite or residue thereof after being administered to a patient in need thereof. Therefore, “the compound of the present invention” mentioned herein also means to encompass various derivative forms of the compound as mentioned above.

A pharmaceutically acceptable salt of the compound of the present invention includes an acid addition salt and a base addition salt thereof.

A suitable acid addition salt is formed from an acid which forms a pharmaceutically acceptable salt. Specific examples include acetate, adipate, aspartate, benzoate, besylate, bicarbonate/carbonate, bisulfate/sulfate, borate, camphorsulfonate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

A suitable base addition salt is formed from a base which forms a pharmaceutically acceptable salt. Specific examples include aluminum, arginine, benzathine, calcium, choline, diethylamine, diolamine, glycine, lysine, magnesium, meglumine, olamine, potassium, sodium, tromethamine and zinc salts.

For a review on suitable salts, see “Handbook of Pharmaceutical Salts: Properties, Selection, and Use” by Stahl and Wermuth (Wiley-VCH, 2002). The method for preparing a pharmaceutically acceptable salt of the compound of the present invention is known to a person skilled in the art.

As used herein, the term “ester” refers to those derived from the compounds of the various formulae in the present application, which include physiologically-hydrolyzable esters (which may be hydrolyzed under physiological conditions to release the compounds of the present invention in the form of free acids or alcohols). The compound of the present invention itself may be an ester as well.

The compound of the present invention can exist as a solvate (preferably a hydrate), wherein the compound of the present invention contains a polar solvent, in particular water, methanol or ethanol for example, as a structural element of the crystal lattice of the compound. The amount of the polar solvent, in particular water, may exist in a stoichiometric or non-stoichiometric ratio.

As can be appreciated by a person skilled in the art, not all nitrogen containing heterocycles can form N-oxides since the nitrogen requires an available lone-pair electron for oxidation to the oxide; a person skilled in the art will recognize those nitrogen containing heterocycles which can form N-oxides. A person skilled in the art will also recognize that tertiary amines can form N-oxides. Synthetic methods for the preparation of N-oxides of heterocycles and tertiary amines are well known to a person skilled in the art, and they include the oxidation of heterocycles and tertiary amines with peroxy acids such as peracetic acid and m-chloroperbenzoic acid (MCPBA), hydrogen peroxide, alkyl hydroperoxides such as tent-butyl hydroperoxide, sodium perborate, and dioxiranes such as dimethyldioxirane. These methods for the preparation of N-oxides have been extensively described and reviewed in literatures, see e.g., T. L. Gilchrist, Comprehensive Organic Synthesis, vol. 7, pp 748-750; A. R. Katritzky and A. J. Boulton, Eds., Academic Press; and G. W. H. Cheeseman and E. S. G. Werstiuk, Advances in Heterocyclic Chemistry, vol. 22, pp 390-392, A. R. Katritzky and A. J. Boulton, Eds., Academic Press.

The metabolite of the compound of the present invention, namely a substance formed in vivo upon administration of the compound of the present invention, is also included within the scope of the present invention. Such a product may result e.g., from the oxidation, reduction, hydrolysis, amidation, de-amidation, esterification, enzymolysis, and the like, of the administered compound. Accordingly, the present invention encompasses the metabolite of the compound of the present invention, including a compound produced by a method comprising contacting the compound of the present invention with a mammal for a period of time sufficient to result in a metabolic product thereof.

Also within the scope of the present invention is a prodmg of the compound of the invention, which is certain derivative of the compound of the invention that may have little or no pharmacological activity itself, but can, when administered into or onto the body, be converted into the compound of the invention having the desired activity, for example, by hydrolytic cleavage. In general, such prodmg will be a functional derivative of the compound which is readily converted in vivo into the compound with desired therapeutic activity. Further information on the use of the prodmg may be found in “Pro-drugs as Novel Delivery Systems”, Vol. 14, ACS Symposium Series (T. Higuchi and V. Stella). The prodrug in accordance with the invention can, for example, be produced by replacing appropriate functionalities present in the compound of the present invention with certain moieties known to those skilled in the art as “pro-moieties” as described, for example, in “Design of Prodrugs” by H. Bundgaard (Elsevier, 1985).

The present invention further encompasses the compound of the present invention having a protecting group. During any of the processes for preparation of the compound of the present invention, it may be necessary and/or desirable to protect sensitive or reactive groups on any of the molecules concerned, thereby resulting in the chemically protected form of the compound of the present invention. This may be achieved by means of conventional protecting groups, e.g., those described in T. W. Greene & P. G. M. Wuts, Protective Groups in Organic Synthesis, John Wiley & Sons, 1991, which is incorporated herein by reference. The protecting groups may be removed at a convenient subsequent stage using methods known from the art.

The term “about” refers to a range within ±10%, preferably within ±5%, and more preferably within ±2% of the specified value.

As used herein, the term “effective amount” refers to the amount of a compound being administered which will relieve to some extent one or more of the symptoms of the disorder being treated.

Unless otherwise indicated, the term “treating” or “treatment”, as used herein, means reversing, alleviating, inhibiting the progress of, or preventing the disorder or condition to which such term applies, or one or more symptoms of such disorder or condition.

As used herein, the “treatment of cough” or “treating cough” means any therapy that reduces the number and/or the severity of cough. Preferably, it means a reduction in the number of coughs, i.e., a direct antitussive effect that reduces the body's urge to cough. In preferred embodiments, compared with the control, the method of the present invention reduces the number of coughs by 40%-95%, preferably 50%-90% in a same period of time.

As used herein, the term “subject” includes a human or non-human animal An exemplary human subject includes a human subject having a disease (such as one described herein) (referred to as a patient), or a normal subject. The term “non-human animal” as used herein includes all vertebrates, such as non-mammals (e.g., birds, amphibians, reptiles) and mammals, such as non-human primates, livestock and/or domesticated animals (such as sheep, dog, cat, cow, pig and the like).

### MODE OF CARRYING OUT THE INVENTION

In some embodiments, the present invention provides a method for treating, suppressing or alleviating cough or cough impulse, comprising administering to a subject in need thereof a therapeutically effective amount of a compound of Formula (I) or a pharmaceutically acceptable salt, ester, stereoisomer, polymorph, solvate, N-oxide, isotopically labeled compound, metabolite or prodrug thereof:

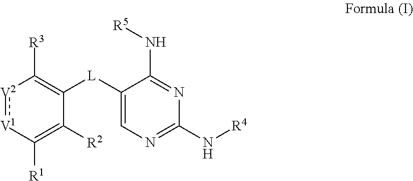

wherein:

L is selected from the group consisting of C(═O), CRR′, NR, O, S, S═O and S(═O)₂;

V¹ is selected from the group consisting of N,

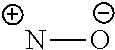

### and NR;

V² is selected from the group consisting of CR⁶ and C(═O);

[symbol] represents either a single bond or a double bond, provided that when [symbol] is a single bond, V¹ is NR and V² is C(═O);

R and R′ are each independently selected from the group consisting of H, halogen, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);

R¹, R², R³ and R⁶ are each independently selected from the group consisting of H, halogen, —CN, —NO₂, —NH₂, —OH, —SH, —Se—R, —Si(R)₃, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl, C₆₋₁₂ aralkyl, C₁₋₆ haloalkyl, —C(═O)R^{a}, —OC(═O)R^{a}, —C(═O)OR^{a}, —OR^{a}, —SR^{a}, —S(═O)R^{a}, —S(═O)₂R^{a}, —S(═O)₂NR^{a}R^{b}, —S(═O)(═NR)R^{a}, —NR^{a}R^{b}, —C(═O)NR^{a}R^{b}, —C(═S)NR^{a}R^{b}, —C(═NR)NR^{a}R^{b}, —NR^{a}—C(═O)R^{b}, —NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-OR^{a}, —C₁₋₆ alkylene-C(═O)R, —C₁₋₆ alkenylene-OR^{a}, —O—C₁₋₆ alkylene-NR^{a}R^{b} and —P(═O)R^{a}R^{b};

R⁴ and R⁵ are each independently selected from the group consisting of H, —C(═O)OR^{a}, —NR^{a}R^{b}, —NR^{a}—C(═O)R^{b}, —NR^{a}—C(═O)OR^{b}, —C₁₋₆ alkylene-NR^{a}R^{b}, —C₁₋₆ alkylene-OR^{a}, —C₁₋₆ alkylene-O—C₁₋₆ alkylene-OR^{a}, C₁₋₆ alkyl, C₁₋₆ haloalkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl;

alternatively, R¹ and R⁴ together form —NH—(C₁₋₆ alkylene)-L-(C₁₋₆ alkylene)-, preferably —NHCH₂CH₂—O—CH₂CH₂—;

the above alkyl, alkylene, alkenyl, alkynyl, cyclic hydrocarbyl, heterocyclyl, aryl, heteroaryl and aralkyl, at each occurrence, are each optionally substituted with one or more substituents independently selected from the group consisting of halogen, hydroxyl, oxo, amino, cyano, nitro, —Si(R)₃, C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl, C₆₋₁₂ aralkyl, —C(═O)R^{a}, —OC(═O)R^{a}, —C(═O)OR^{a}, —OR^{a}, —SR^{a}, —S(═O)R^{a}, —S(═O)₂R^{a}, —S(═O)₂NR^{a}R^{b}, —NR^{a}R^{b}, —C(═O)NR^{a}R^{b}, —NR^{a}—C(═OR^{b}, —NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-OR^{a}, —C₁₋₆ alkenylene-OR^{a} and —O—C₁₋₆ alkylene-NR^{a}R^{b}, the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, —NR^{a}R^{b}, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl; and

R^{a} and R^{b}, at each occurrence, are each independently selected from the group consisting of H, —OH, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl; alternatively, R^{a} nd R^{b} together with the atom to which they are attached form a 3- to 12-membered heterocycle or heteroaromatic ring, the above groups are further optionally substituted with one or more substituents independently selected from the group consisting of halogen, hydroxyl, oxo, amino, cyano, nitro, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl.

In other embodiments, the present invention provides use of a compound of above Formula (I) or a pharmaceutically acceptable salt, ester, stereoisomer, polymorph, solvate, N-oxide, isotopically labeled compound, metabolite or prodrug thereof in the manufacture of a medicament for treating, suppressing or alleviating cough or cough impulse.

In other embodiments, the present invention provides a compound of above Formula (I) or a pharmaceutically acceptable salt, ester, stereoisomer, polymorph, solvate, N-oxide, isotopically labeled compound, metabolite or prodrug thereof for use of treating, suppressing or alleviating cough or cough impulse.

In preferred embodiments, L is selected from the group consisting of CH₂, O, S and NH.

In preferred embodiments, V¹ is selected from the group consisting of N,

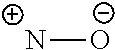

### and NCH₃.

In preferred embodiments, V² is selected from the group consisting of CH, C—NHCH₃, C—OCH₃, C—F and C(═O).

In preferred embodiments, R^{a} and R^{b}, at each occurrence, are each independently selected from the group consisting of H, —OH, methyl, ethyl, n-propyl, isopropyl, cyclopropyl, phenyl, benzyl, methoxy and ethoxy; alternatively, R^{a} and R^{b} together with the atom to which they are attached form a 5- to 8-membered heterocycle or heteroaromatic ring.

In preferred embodiments, R², R³ and R⁶ are each independently selected from the group consisting of H, F, Cl, Br, I, —CN, —NH₂, —OH, —SH, —Se—CH₃, —Si(CH₃)₃, —CH₂NH₂, —CH₂NHCH₃, —CH₂N(CH₃)₂, methyl, ethyl, n-propyl, isopropyl, n-butyl, isobutyl, tent-butyl, cyclopropyl, cyclobutyl, cyclopentyl, cyclohexyl, vinyl, propenyl, allyl, ethynyl, propynyl, trifluoromethyl, acetyl, —C(═O)OH, —C(═O)NH₂, —C(═S)NH₂, —C(═NH)NH₂, —NHCH₃, —NHCH₂CH₃, —NHCH₂CF₃—N(CH₃)₂, —N(CH₃)(C₂H₅), —N(C₂H₅)₂, —NHCH₂CH₂OH, —NH—C(═O)CH₃, —NH—C(═O)CH═CH₂, methoxy, ethoxy, propoxy, phenyl, —NH—C(═O)—NH₂, —NH—C(═O)OCH₃, —SCH₃, —SCH₂CH₃, —SC(CH₃)₃, —SBn, —S(═O)CH₃, —S(═O)Bn, —S(═O)₂CH₃, —S(═O)₂Bn, —S(═O)₂NH₂, —S(═O)2NHCH₃, —S(═O)₂N(CH₃)₂, —S(═O)(═NH)CH₃, —P(═O)(CH₃)₂, —P(═O)(C₂H₅)₂,

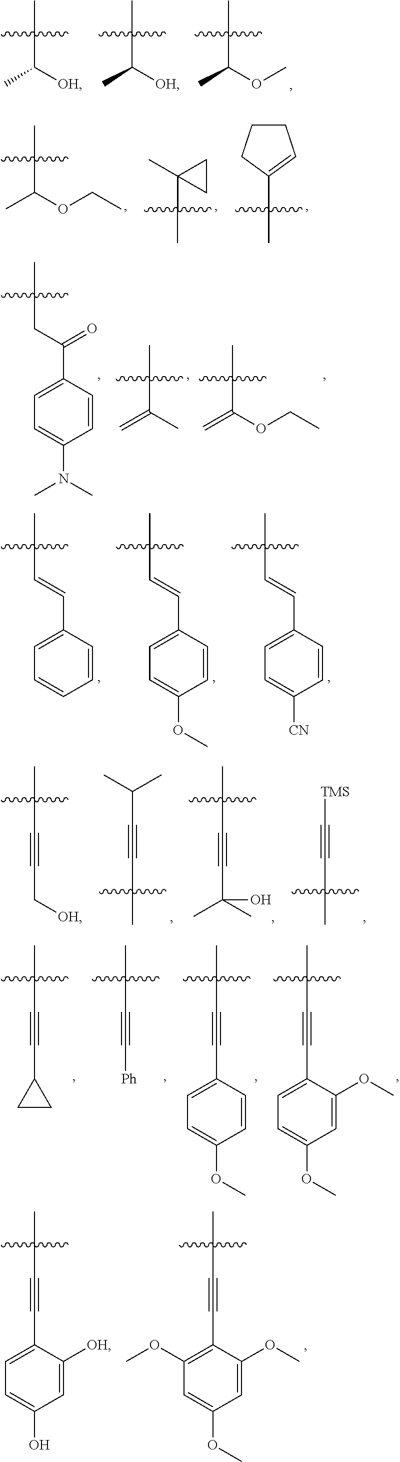

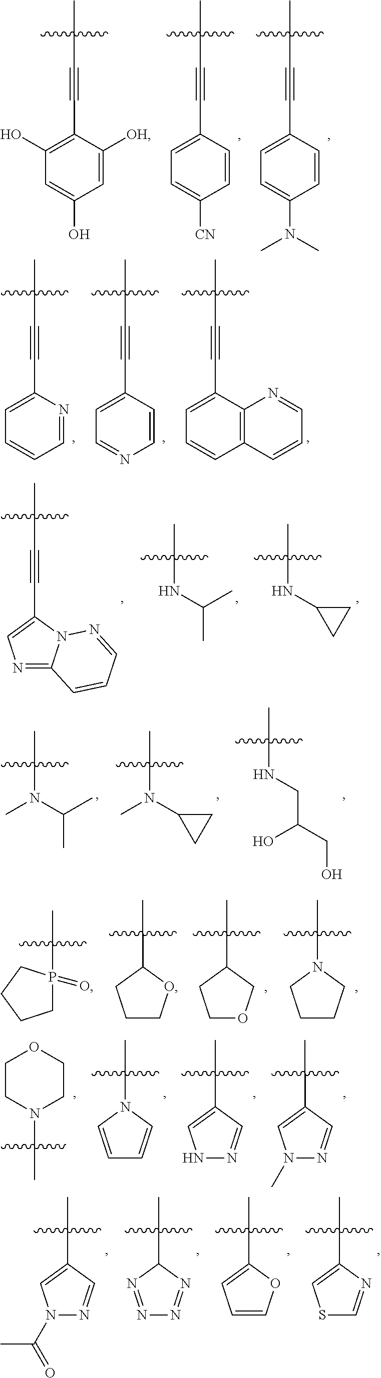

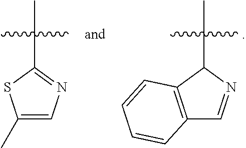

In preferred embodiments, R⁴ and R⁵ are each independently selected from the group consisting of H, —C(═O)OC(CH₃)₃, —NH₂, —NHCH₃, —NHPh, —NHC(═O)CH₃, —NHBoc, methyl, ethyl, —CH₂CF₃, n-propyl, isopropyl, n-butyl, isobutyl, tent-butyl, cyclopropyl, cyclobutyl, cyclopentyl, cyclohexyl, phenyl, benzyl,

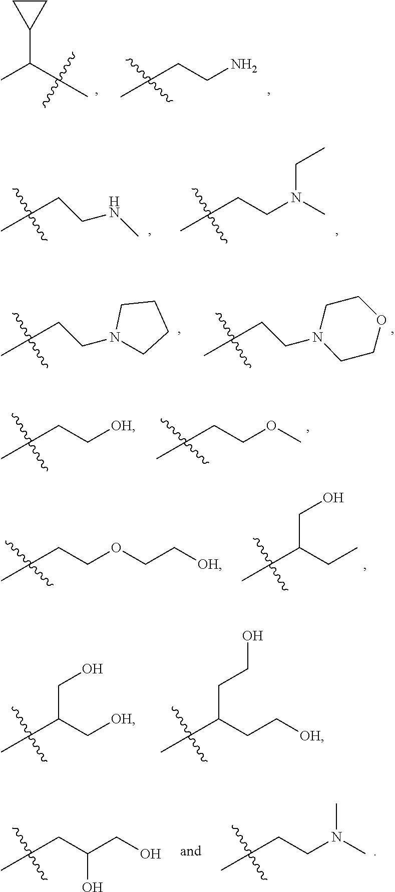

In preferred embodiments, the compound of Formula (I) has the structure of any of the following formulae:

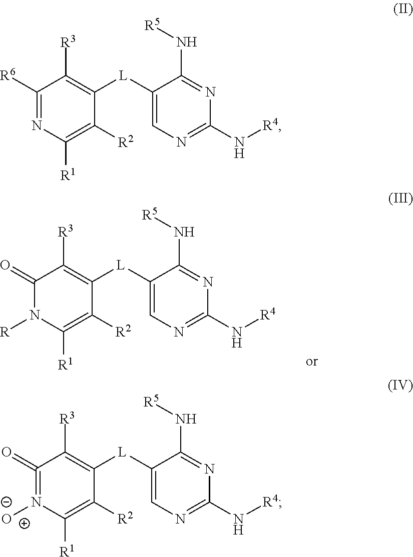

preferably has the structure of any of the following formulae:

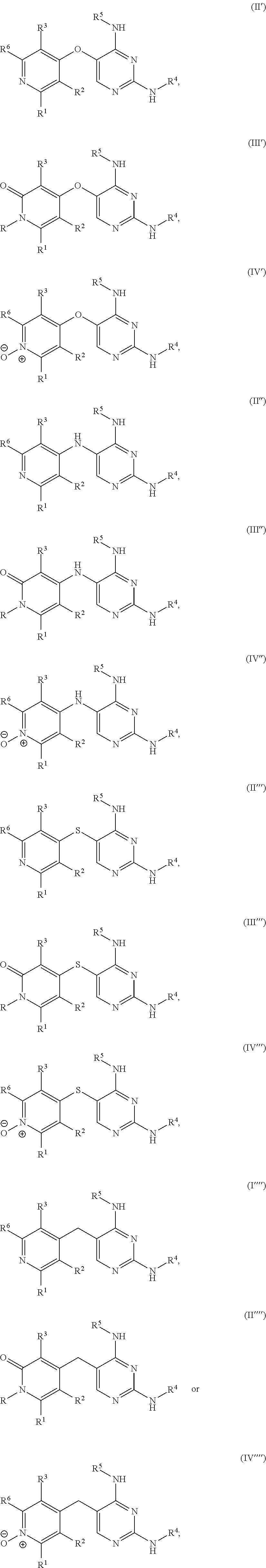

more preferably, the compound of Formula (I) has the structure of any of the following formulae:

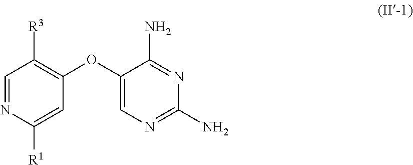

wherein:

R¹ is selected from the group consisting of F, Cl, Br, I and C₂₋₆ alkynyl, preferably Br or ethynyl; and

R³ is C₁₋₆ alkyl, preferably isopropyl.

The technical solution obtained by any combination of the various embodiments is encompassed by the invention.

In preferred embodiments, the compound of Formula (I) has the following structure:

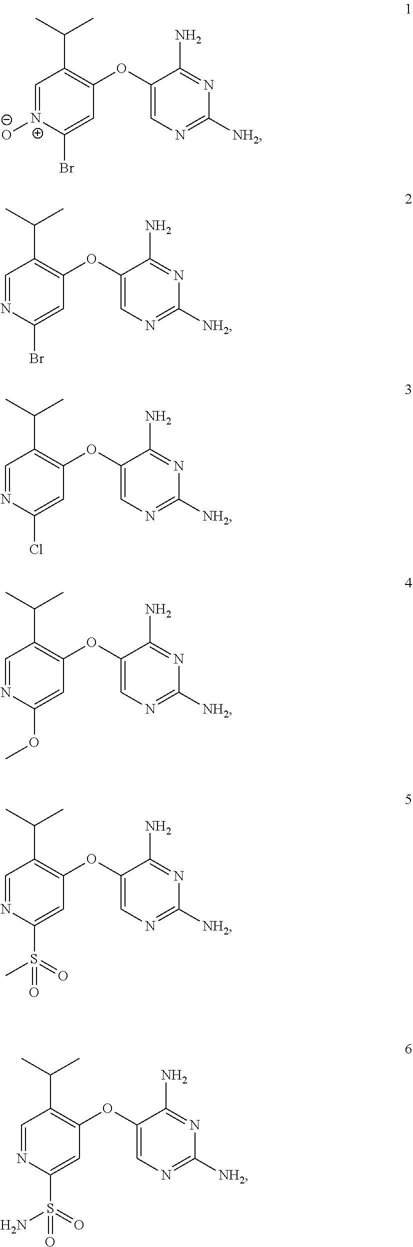

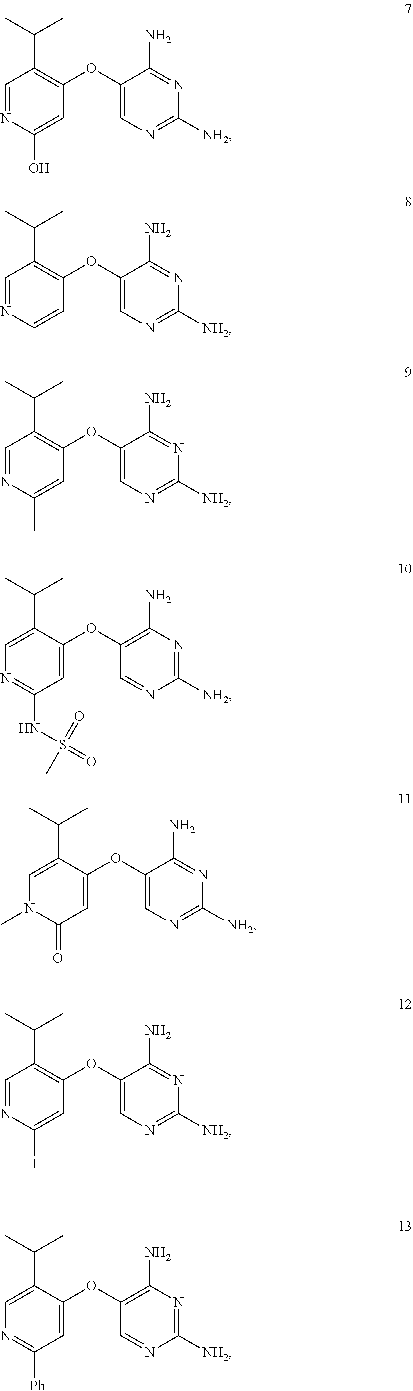

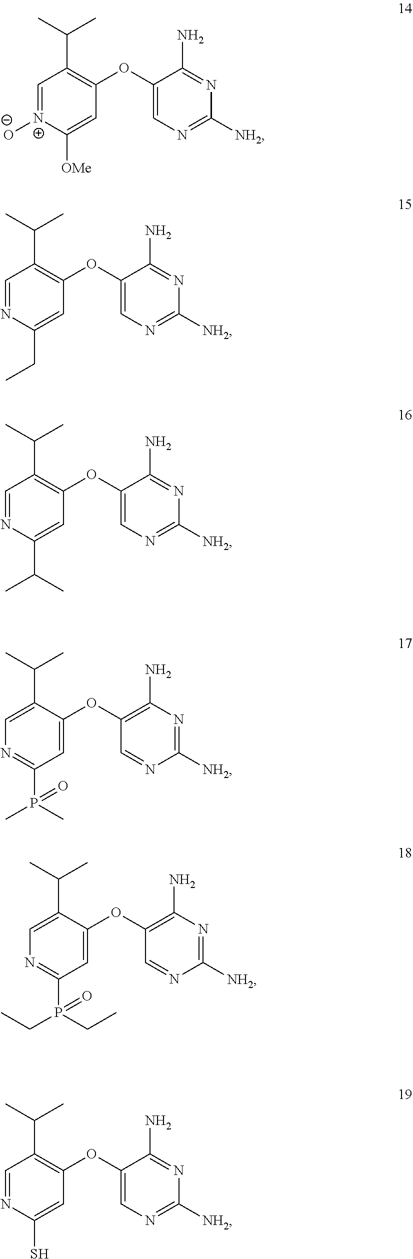

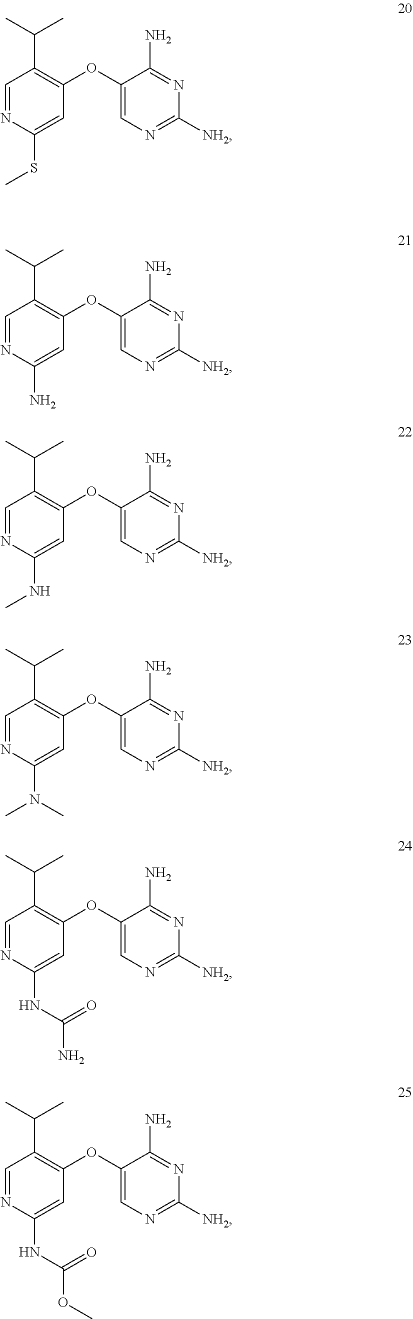

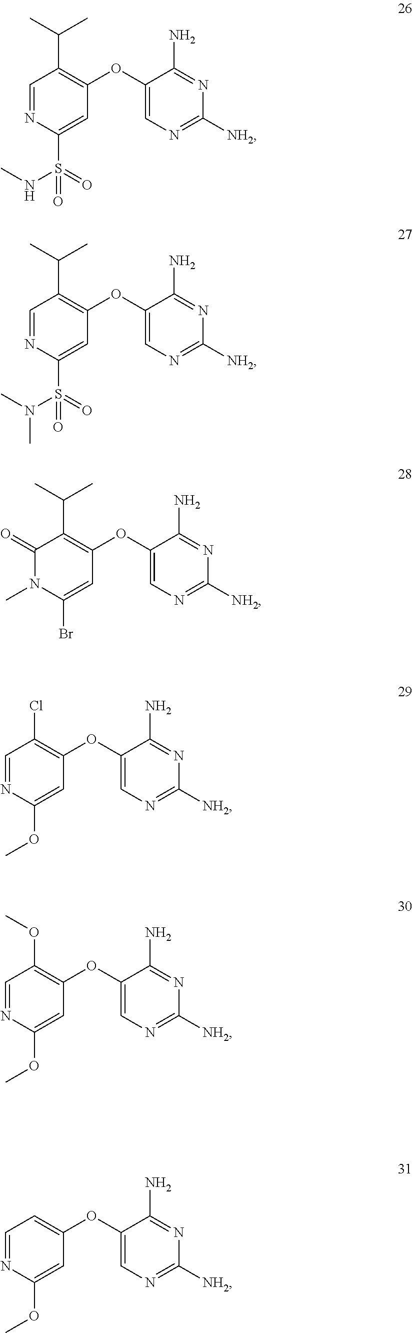

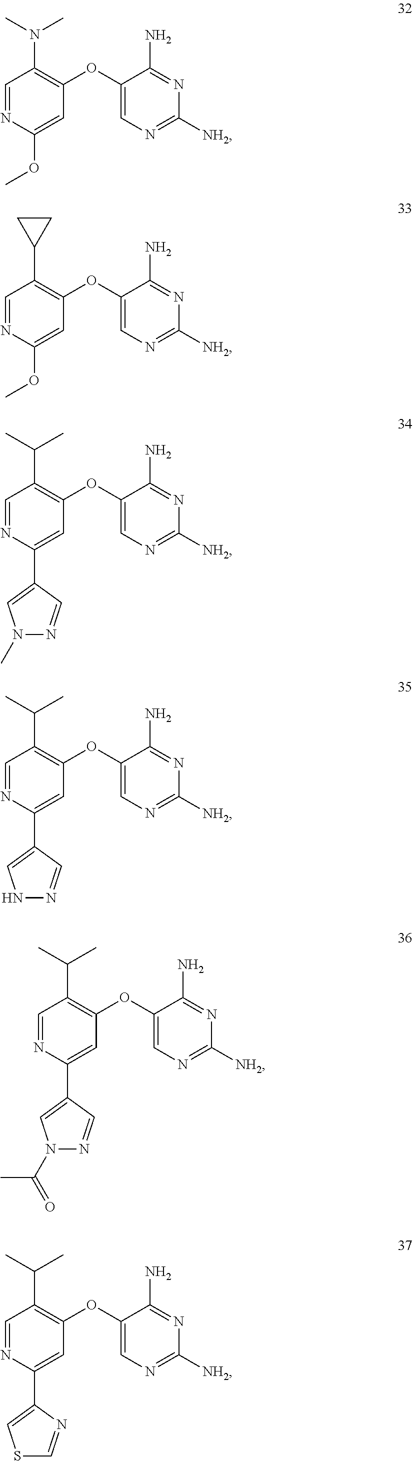

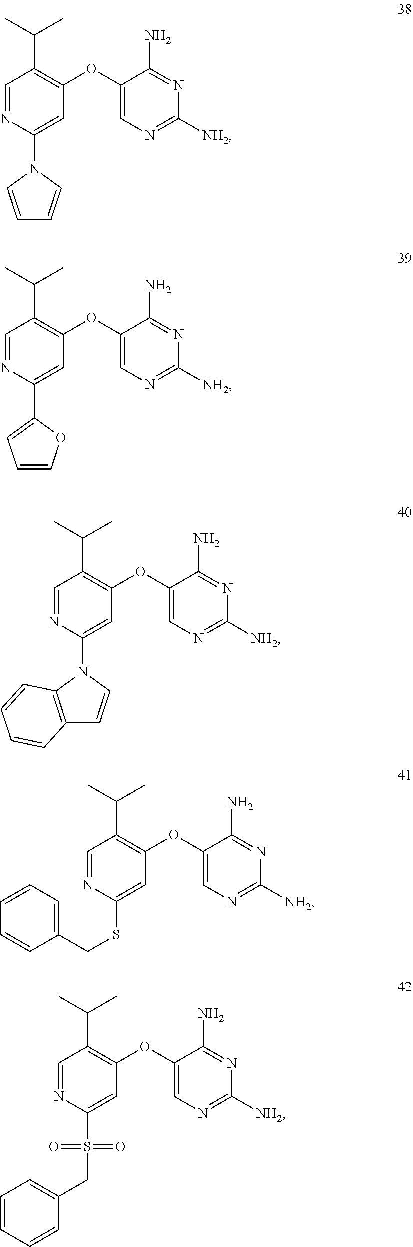 [SMILES: CC(C)c1cnc(-n2cccc2)cc1Oc1cnc(N)nc1N ; CC(C)c1cnc(-c2ccco2)cc1Oc1cnc(N)nc1N ; CC(C)c1cnc(-n2ccc3ccccc32)cc1Oc1cnc(N)nc1N ; CC(C)c1cnc(SCc2ccccc2)cc1Oc1cnc(N)nc1N ; CC(C)c1cnc(S(=O)(=O)Cc2ccccc2)cc1Oc1cnc(N)nc1N]

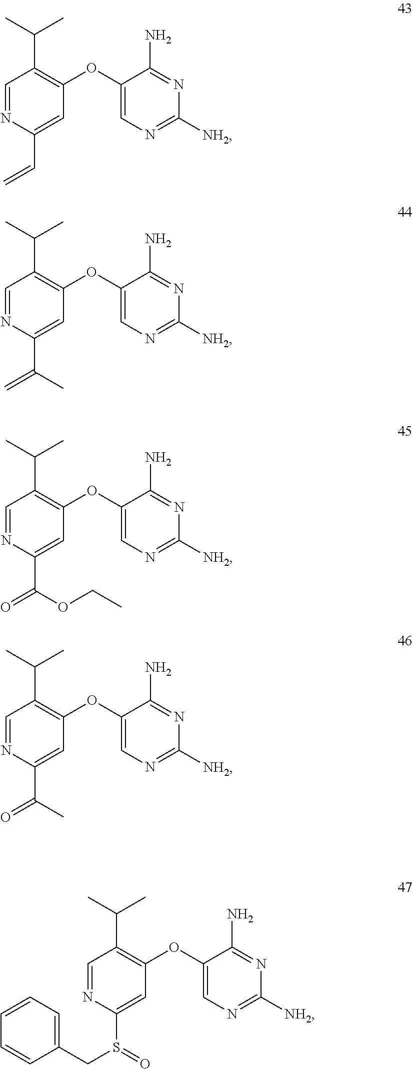

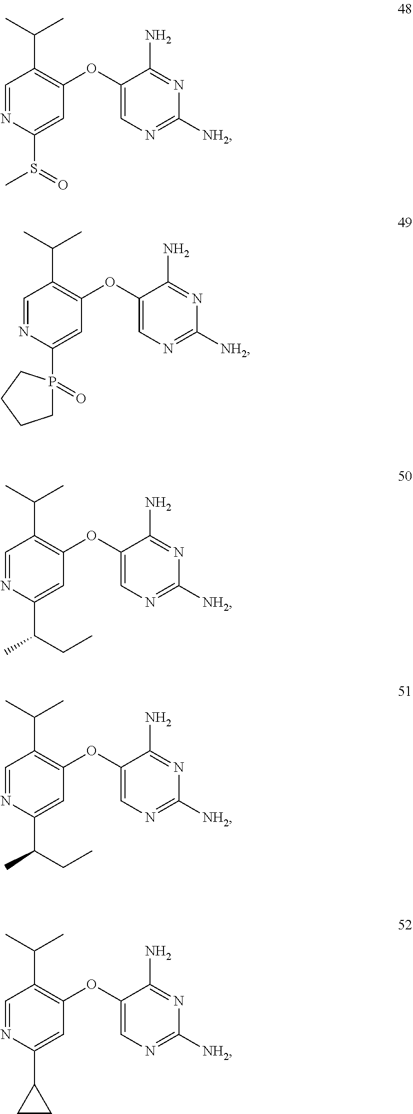 [SMILES: CC(C)c1cnc(S(C)=O)cc1Oc1cnc(N)nc1N ; CC(C)c1cnc(P2(=O)CCCC2)cc1Oc1cnc(N)nc1N ; CC[C@H](C)c1cc(Oc2cnc(N)nc2N)c(C(C)C)cn1 ; CC[C@@H](C)c1cc(Oc2cnc(N)nc2N)c(C(C)C)cn1 ; CC(C)c1cnc(C2CC2)cc1Oc1cnc(N)nc1N]

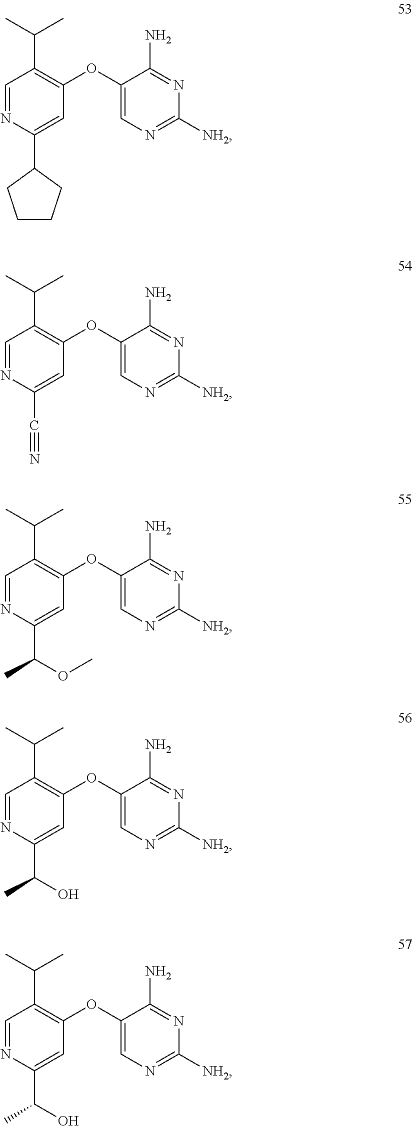 [SMILES: CC(C)c1cnc(C2CCCC2)cc1Oc1cnc(N)nc1N ; CC(C)c1cnc(C#N)cc1Oc1cnc(N)nc1N ; CO[C@@H](C)c1cc(Oc2cnc(N)nc2N)c(C(C)C)cn1 ; CC(C)c1cnc([C@H](C)O)cc1Oc1cnc(N)nc1N ; CC(C)c1cnc([C@@H](C)O)cc1Oc1cnc(N)nc1N]

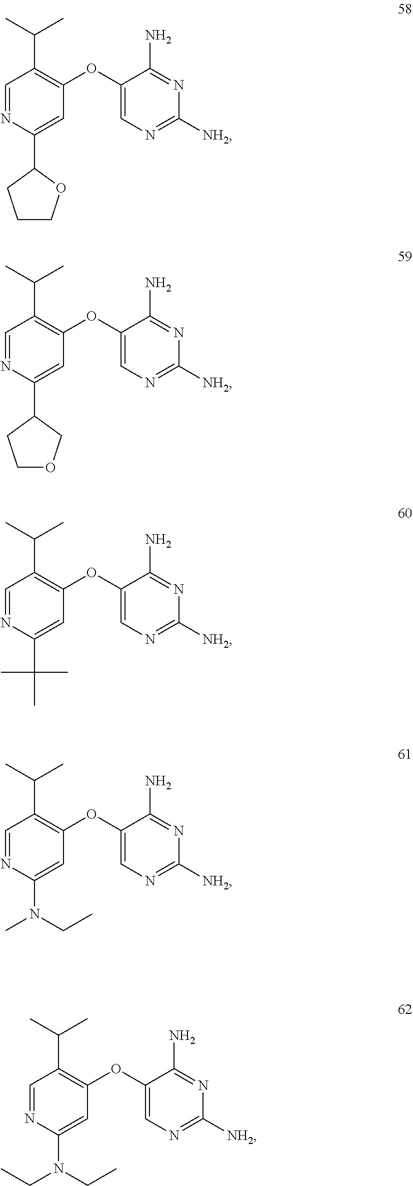 [SMILES: CC(C)c1cnc(C2CCCO2)cc1Oc1cnc(N)nc1N ; CC(C)c1cnc(C2CCOC2)cc1Oc1cnc(N)nc1N ; CC(C)c1cnc(C(C)(C)C)cc1Oc1cnc(N)nc1N ; CCN(C)c1cc(Oc2cnc(N)nc2N)c(C(C)C)cn1 ; CCN(CC)c1cc(Oc2cnc(N)nc2N)c(C(C)C)cn1]

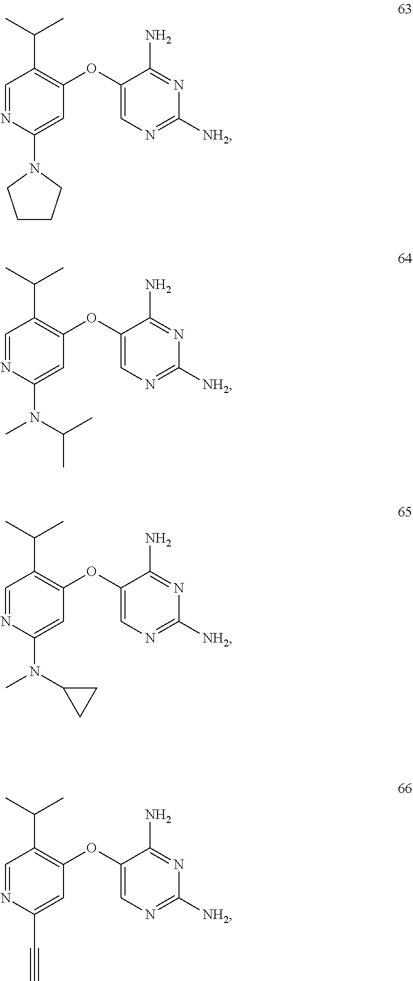 [SMILES: CC(C)c1cnc(N2CCCC2)cc1Oc1cnc(N)nc1N ; CC(C)c1cnc(N(C)C(C)C)cc1Oc1cnc(N)nc1N ; CC(C)c1cnc(N(C)C2CC2)cc1Oc1cnc(N)nc1N ; C#Cc1cc(Oc2cnc(N)nc2N)c(C(C)C)cn1]

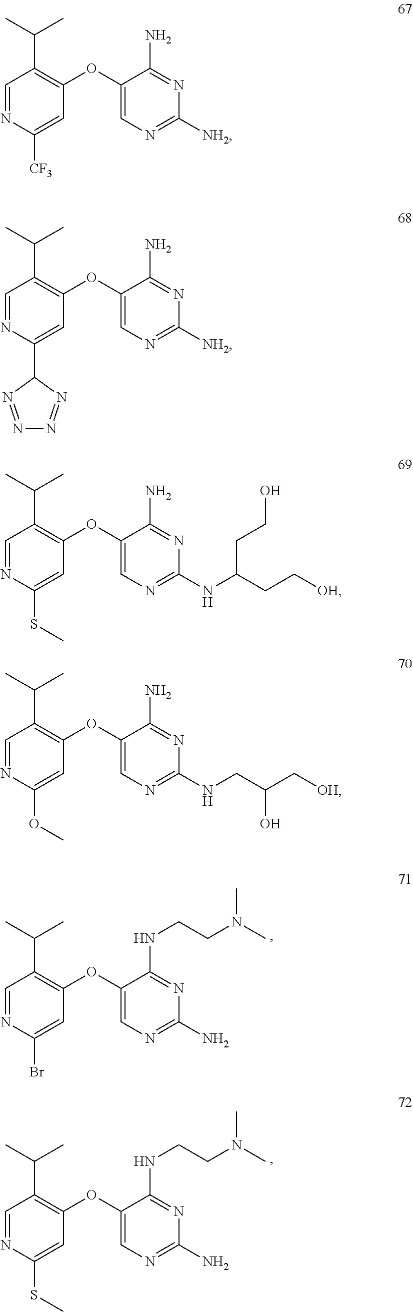

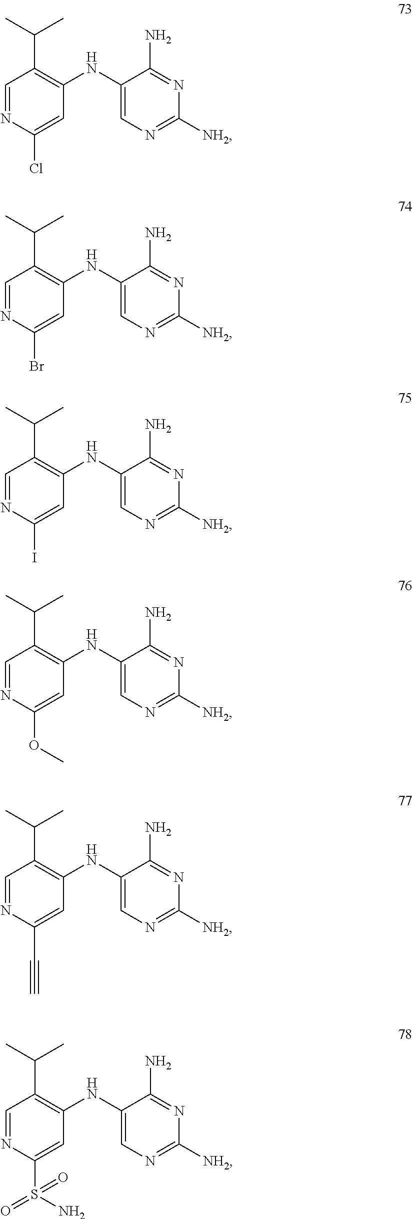 [SMILES: CC(C)c1cnc(Cl)cc1Nc1cnc(N)nc1N ; CC(C)c1cnc(Br)cc1Nc1cnc(N)nc1N ; CC(C)c1cnc(I)cc1Nc1cnc(N)nc1N ; COc1cc(Nc2cnc(N)nc2N)c(C(C)C)cn1 ; C#Cc1cc(Nc2cnc(N)nc2N)c(C(C)C)cn1 ; CC(C)c1cnc(S(N)(=O)=O)cc1Nc1cnc(N)nc1N]

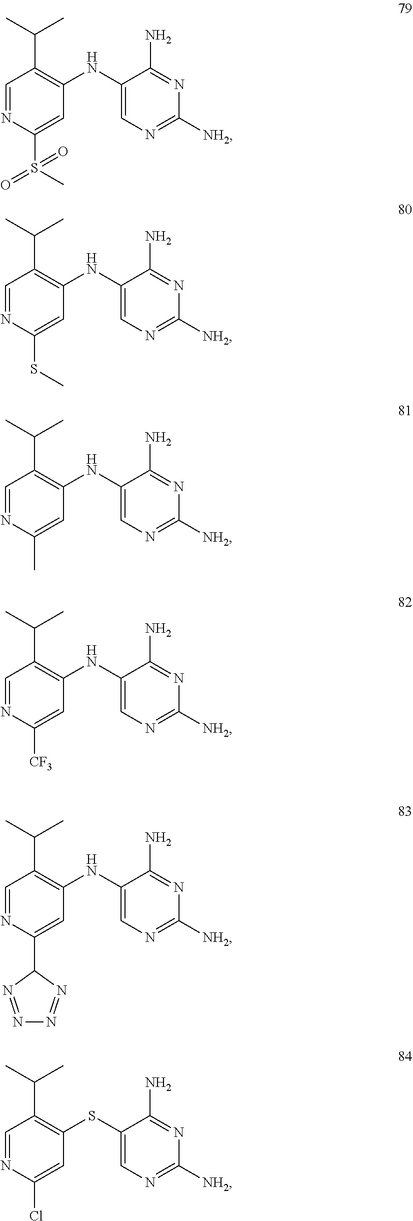

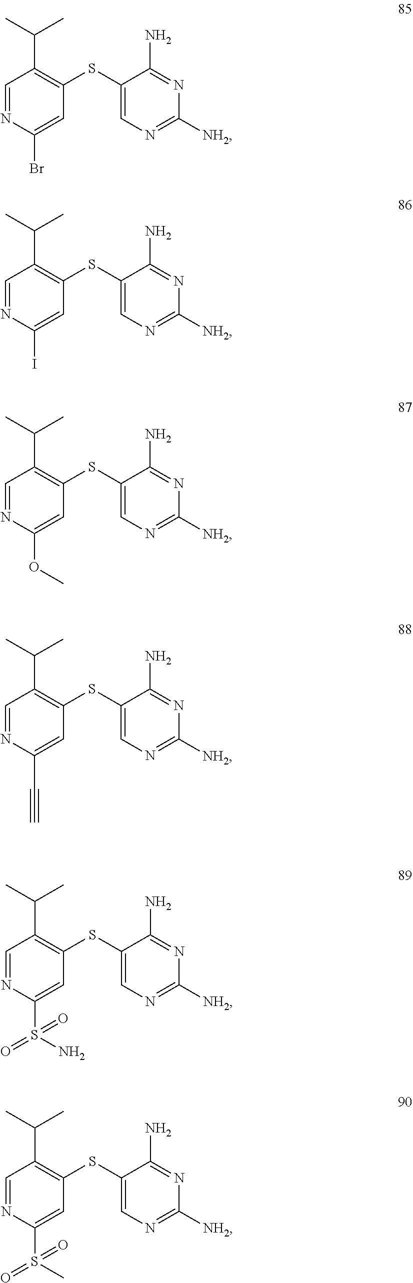 [SMILES: CC(C)c1cnc(Br)cc1Sc1cnc(N)nc1N ; CC(C)c1cnc(I)cc1Sc1cnc(N)nc1N ; COc1cc(Sc2cnc(N)nc2N)c(C(C)C)cn1 ; C#Cc1cc(Sc2cnc(N)nc2N)c(C(C)C)cn1 ; CC(C)c1cnc(S(N)(=O)=O)cc1Sc1cnc(N)nc1N ; CC(C)c1cnc(S(C)(=O)=O)cc1Sc1cnc(N)nc1N]

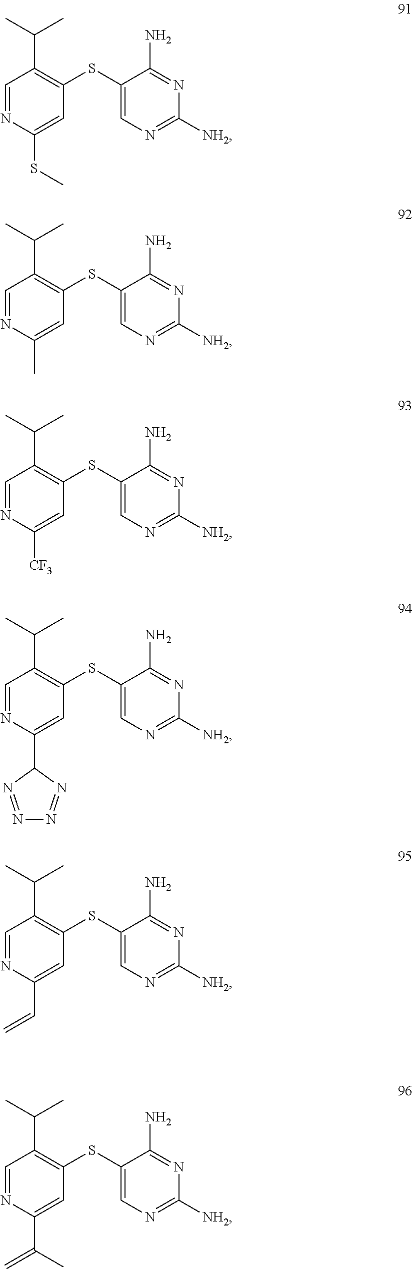

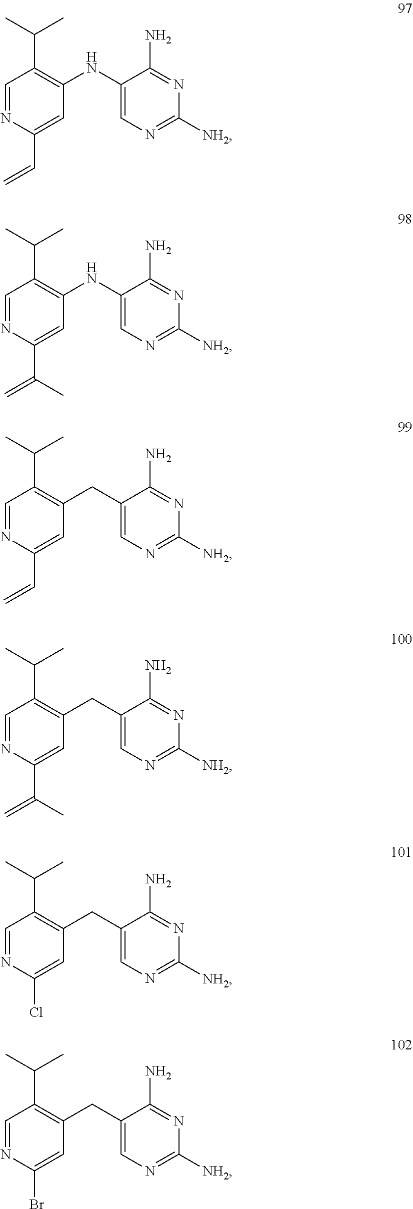

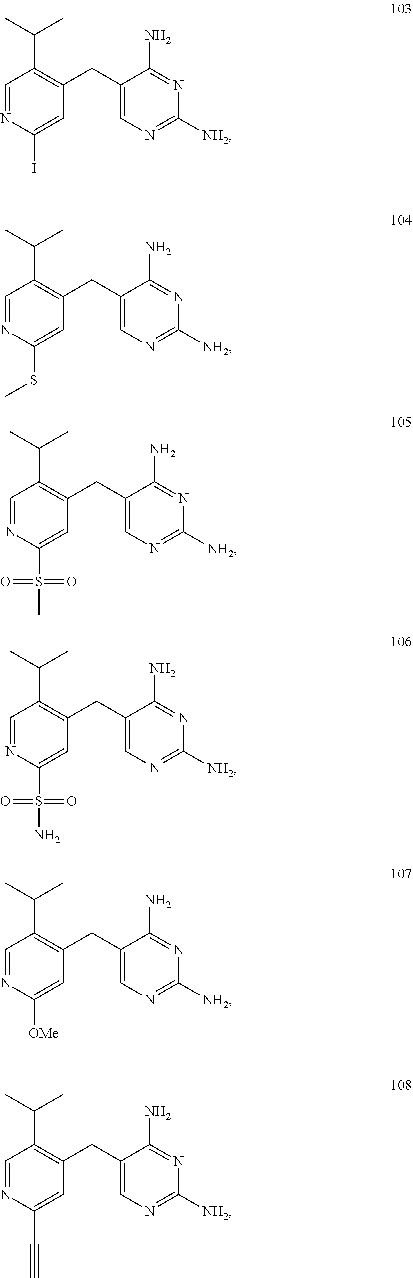

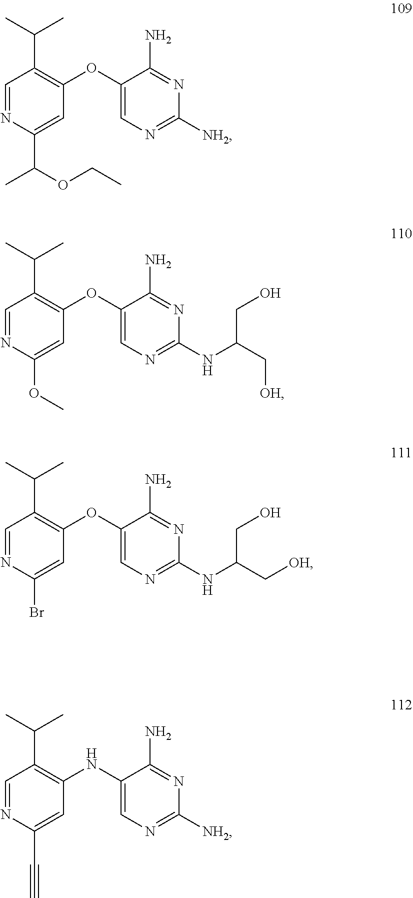 [SMILES: CCOC(C)c1cc(Oc2cnc(N)nc2N)c(C(C)C)cn1 ; COc1cc(Oc2cnc(NC(CO)CO)nc2N)c(C(C)C)cn1 ; CC(C)c1cnc(Br)cc1Oc1cnc(NC(CO)CO)nc1N ; C#Cc1cc(Nc2cnc(N)nc2N)c(C(C)C)cn1]

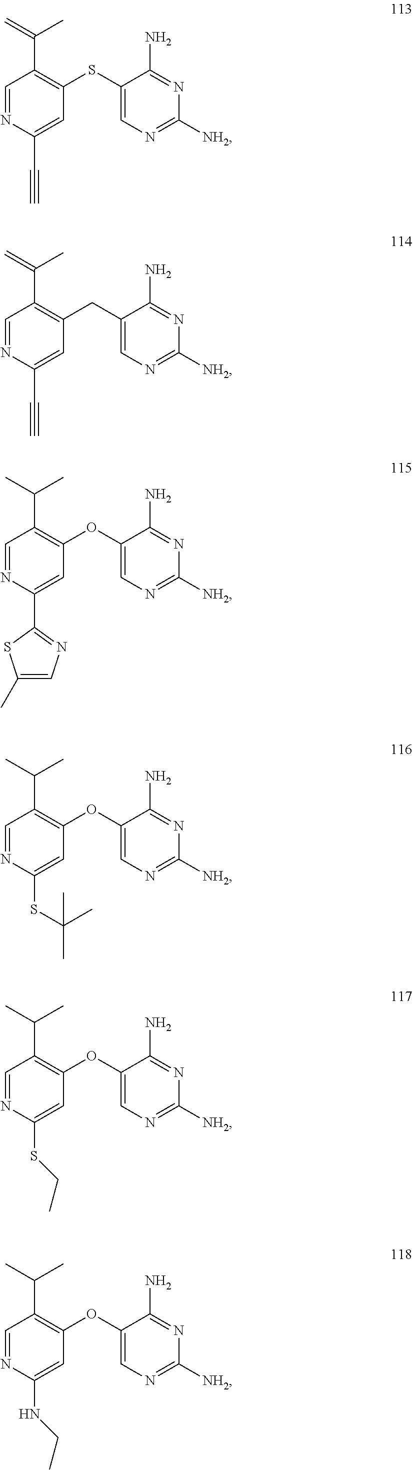

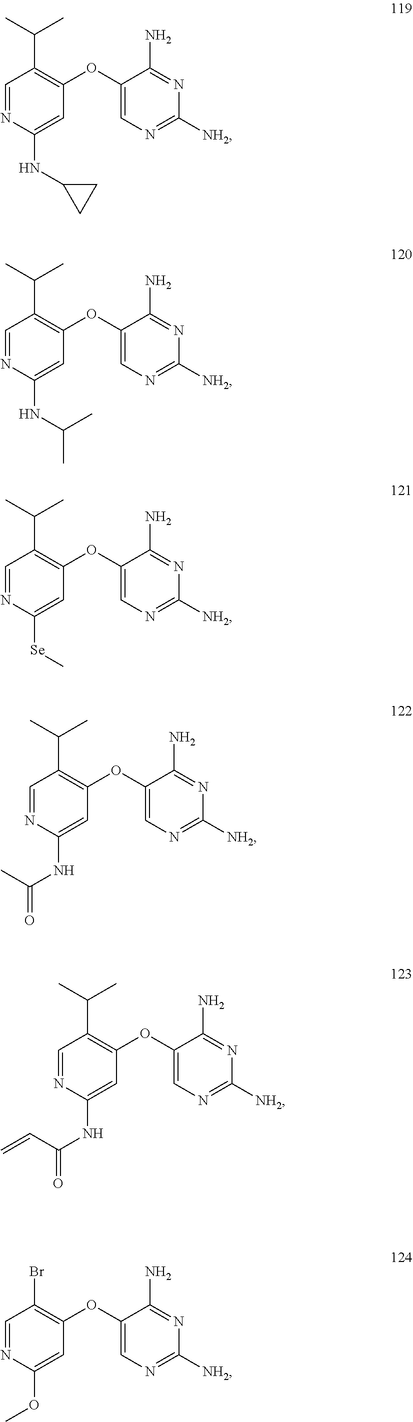

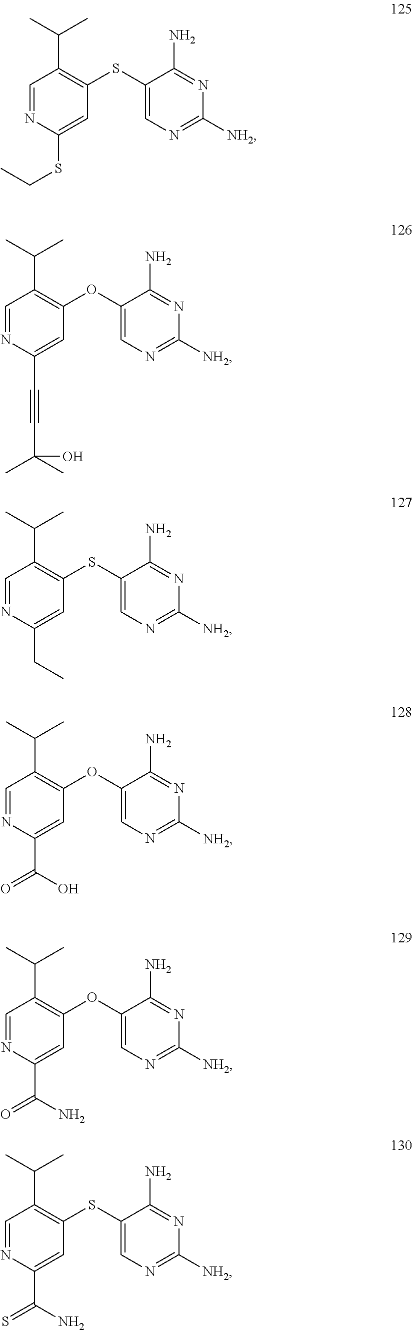

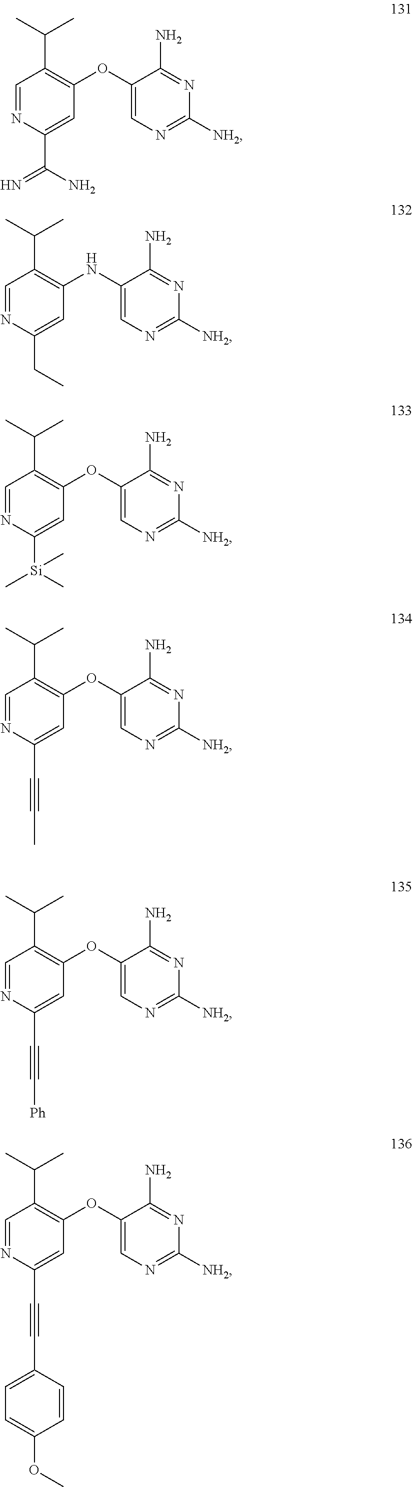

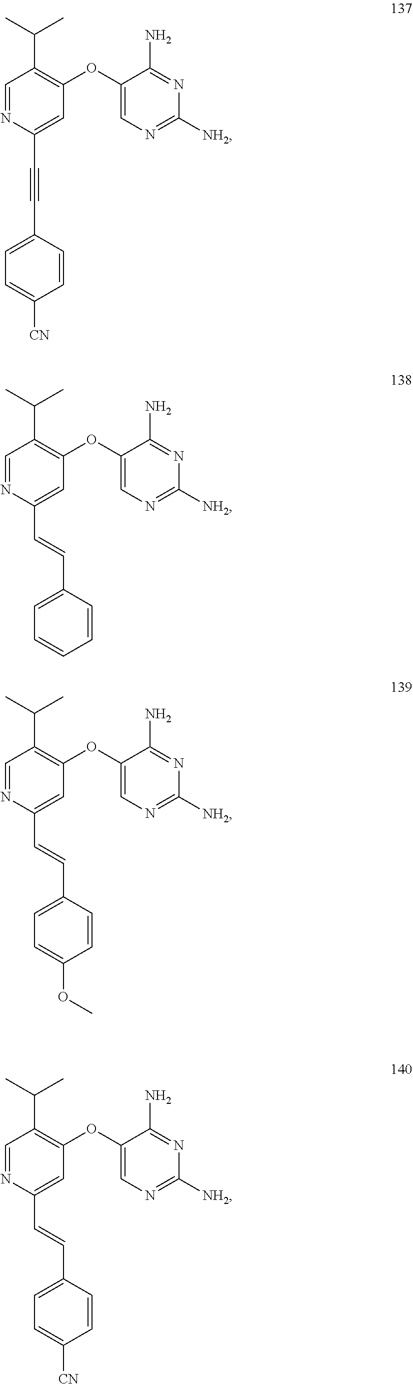

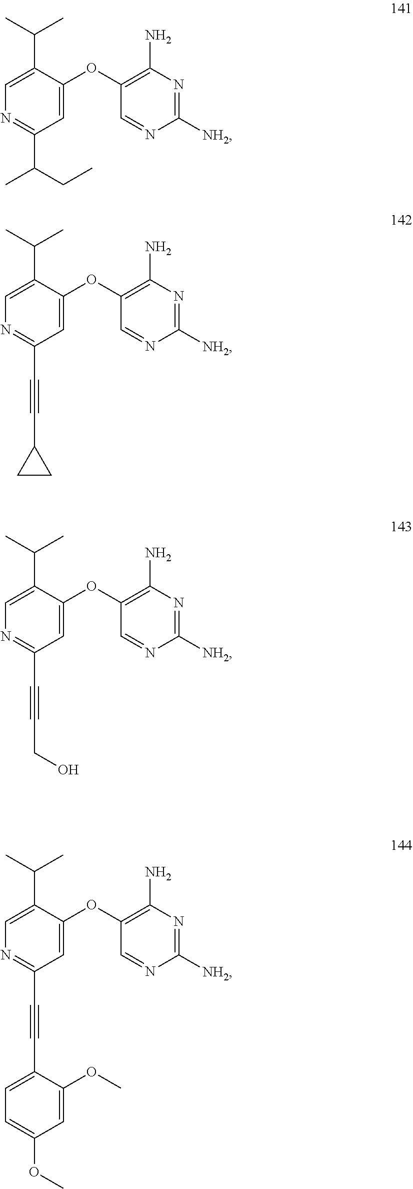

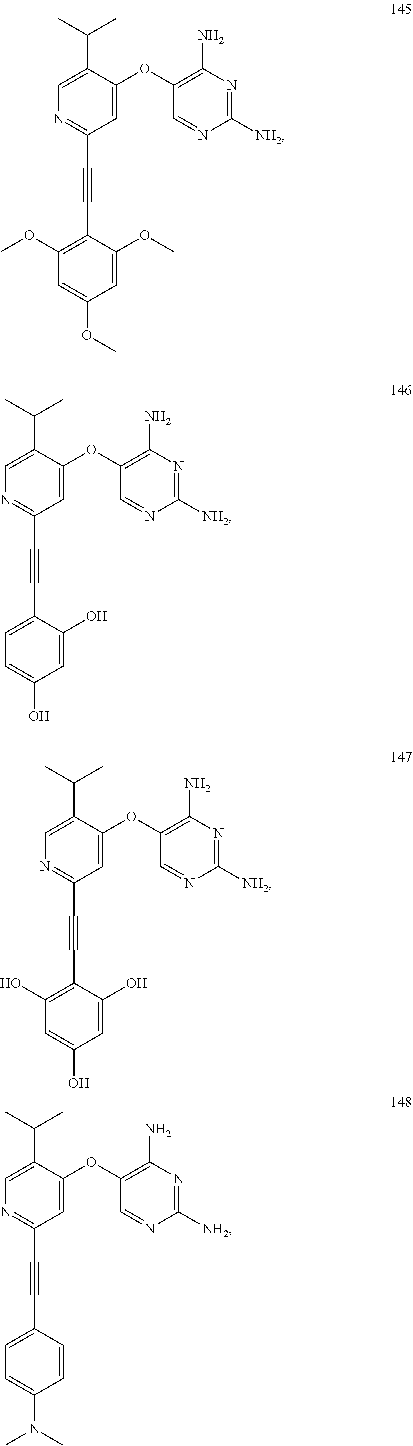

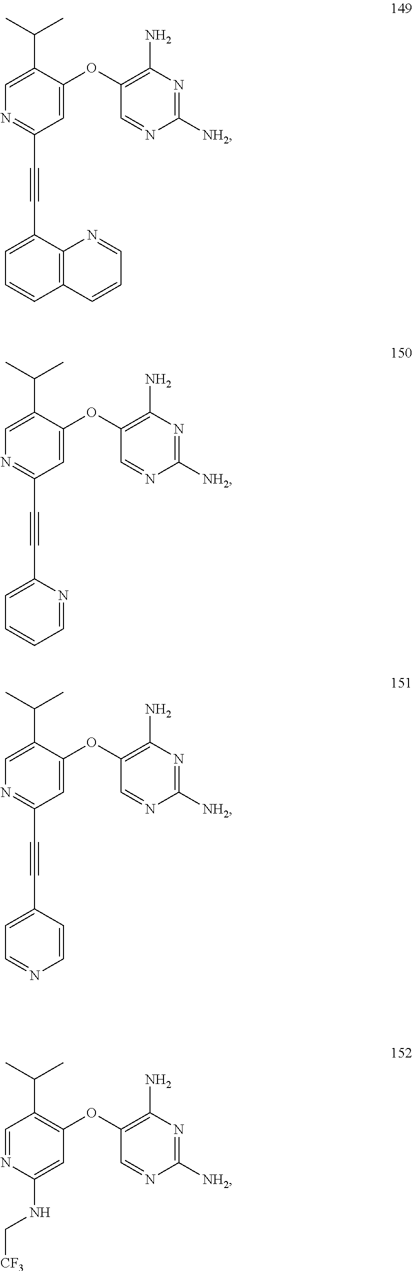 [SMILES: CC(C)c1cnc(C#Cc2cccc3cccnc23)cc1Oc1cnc(N)nc1N ; CC(C)c1cnc(C#Cc2ccccn2)cc1Oc1cnc(N)nc1N ; CC(C)c1cnc(C#Cc2ccncc2)cc1Oc1cnc(N)nc1N ; CC(C)c1cnc(NCC(F)(F)F)cc1Oc1cnc(N)nc1N]

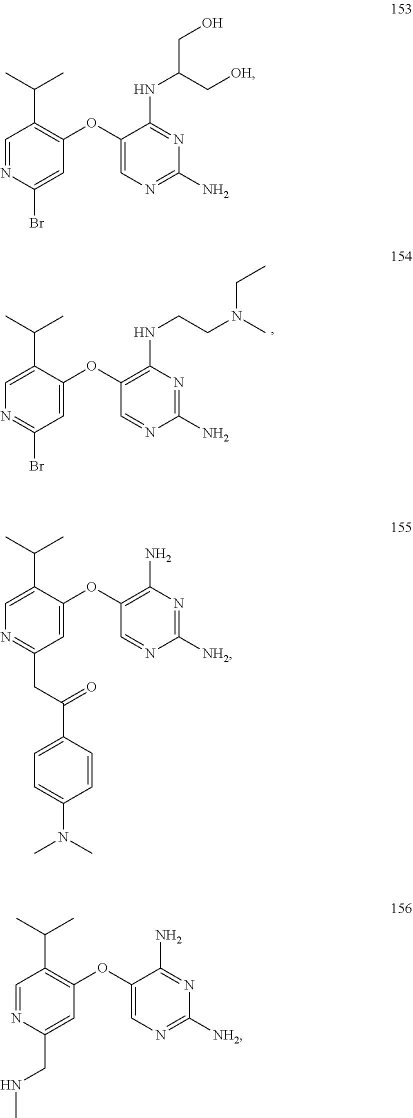 [SMILES: CC(C)c1cnc(Br)cc1Oc1cnc(N)nc1NC(CO)CO ; CCN(C)CCNc1nc(N)ncc1Oc1cc(Br)ncc1C(C)C ; CC(C)c1cnc(CC(=O)c2ccc(N(C)C)cc2)cc1Oc1cnc(N)nc1N ; CNCc1cc(Oc2cnc(N)nc2N)c(C(C)C)cn1]

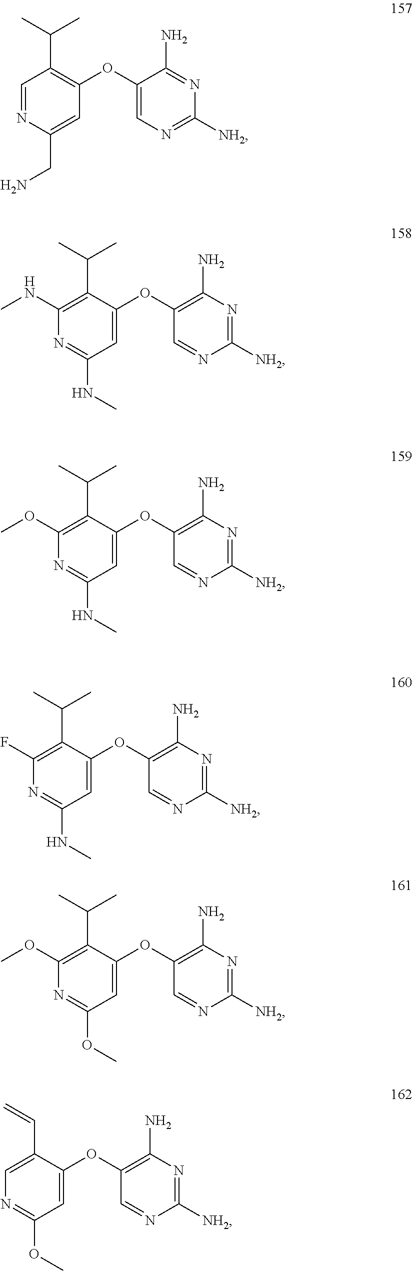

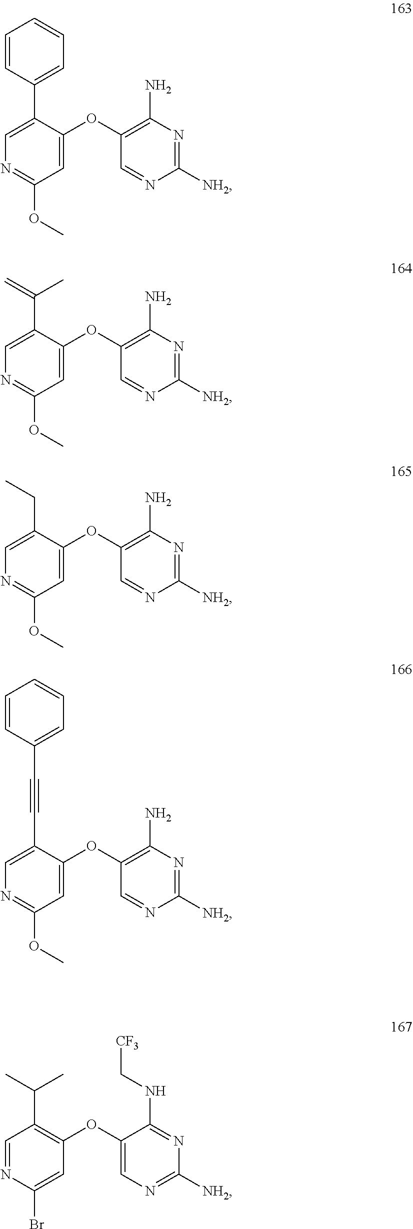

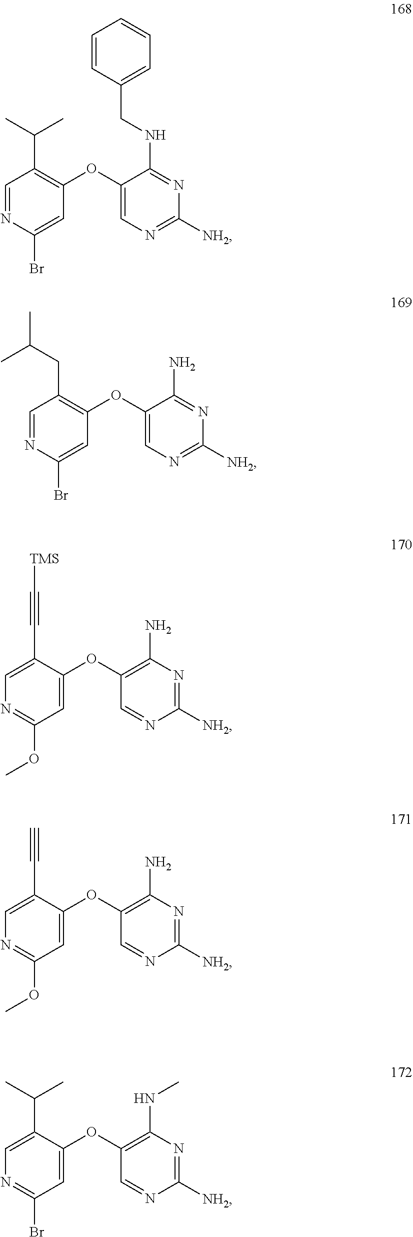

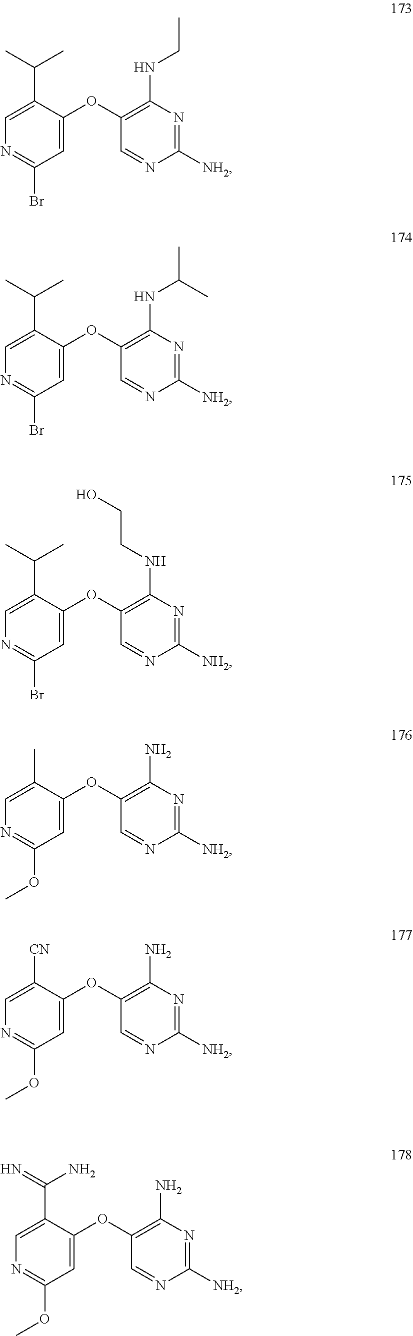 [SMILES: CCNc1nc(N)ncc1Oc1cc(Br)ncc1C(C)C ; CC(C)Nc1nc(N)ncc1Oc1cc(Br)ncc1C(C)C ; CC(C)c1cnc(Br)cc1Oc1cnc(N)nc1NCCO ; COc1cc(Oc2cnc(N)nc2N)c(C)cn1 ; COc1cc(Oc2cnc(N)nc2N)c(C#N)cn1 ; COc1cc(Oc2cnc(N)nc2N)c(C(=N)N)cn1]

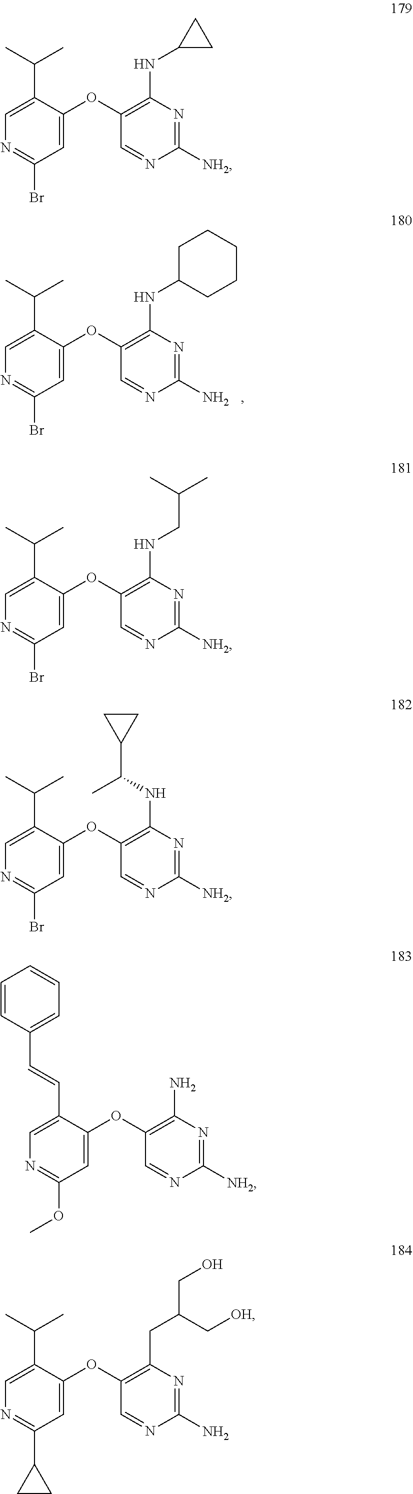

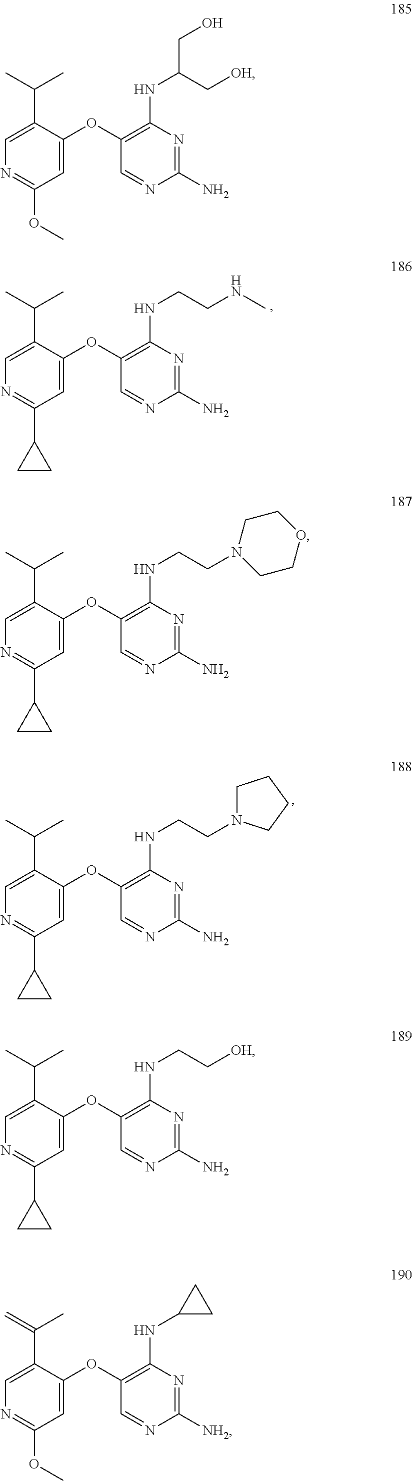 [SMILES: COc1cc(Oc2cnc(N)nc2NC(CO)CO)c(C(C)C)cn1 ; CNCCNc1nc(N)ncc1Oc1cc(C2CC2)ncc1C(C)C ; CC(C)c1cnc(C2CC2)cc1Oc1cnc(N)nc1NCCN1CCOCC1 ; CC(C)c1cnc(C2CC2)cc1Oc1cnc(N)nc1NCCN1CCCC1 ; CC(C)c1cnc(C2CC2)cc1Oc1cnc(N)nc1NCCO ; C=C(C)c1cnc(OC)cc1Oc1cnc(N)nc1NC1CC1]

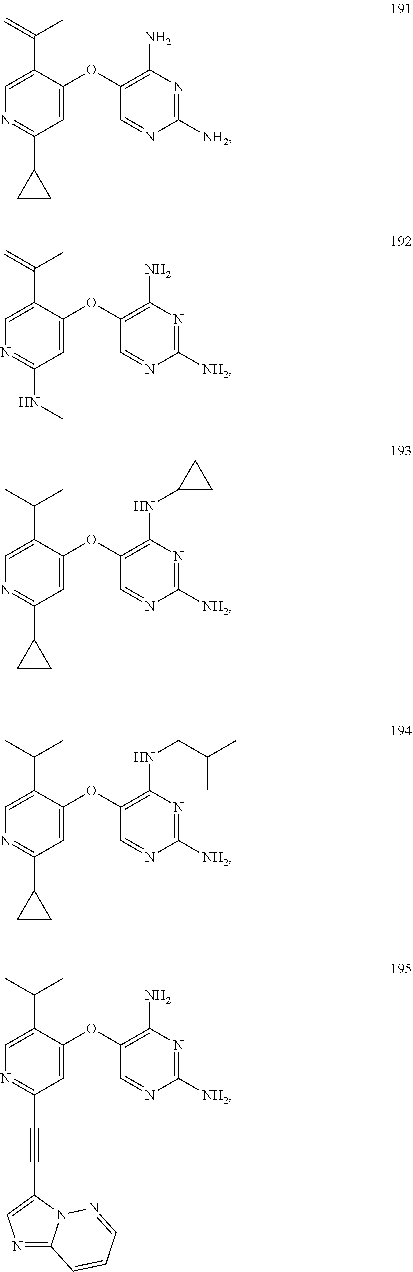 [SMILES: C=C(C)c1cnc(C2CC2)cc1Oc1cnc(N)nc1N ; C=C(C)c1cnc(NC)cc1Oc1cnc(N)nc1N ; CC(C)c1cnc(C2CC2)cc1Oc1cnc(N)nc1NC1CC1 ; CC(C)CNc1nc(N)ncc1Oc1cc(C2CC2)ncc1C(C)C ; CC(C)c1cnc(C#Cc2cnc3cccnn23)cc1Oc1cnc(N)nc1N]

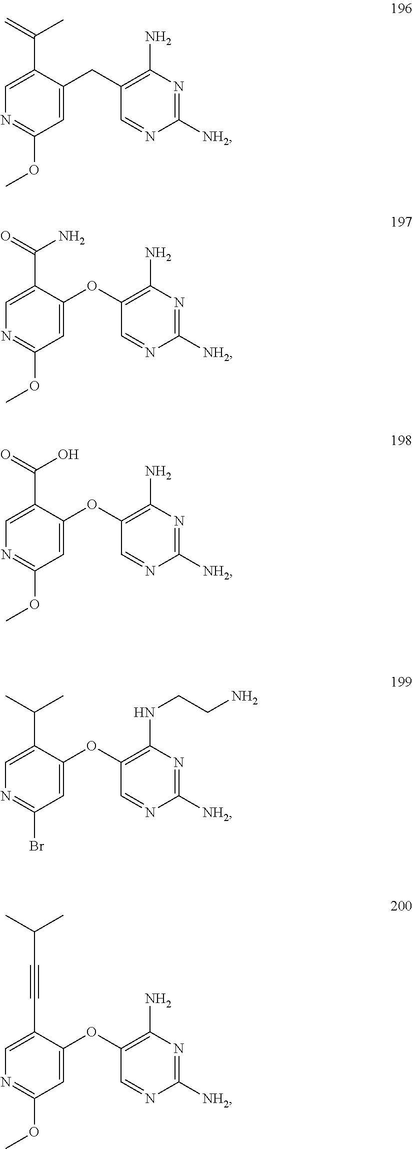 [SMILES: C=C(C)c1cnc(OC)cc1Cc1cnc(N)nc1N ; COc1cc(Oc2cnc(N)nc2N)c(C(N)=O)cn1 ; COc1cc(Oc2cnc(N)nc2N)c(C(=O)O)cn1 ; CC(C)c1cnc(Br)cc1Oc1cnc(N)nc1NCCN ; COc1cc(Oc2cnc(N)nc2N)c(C#CC(C)C)cn1]

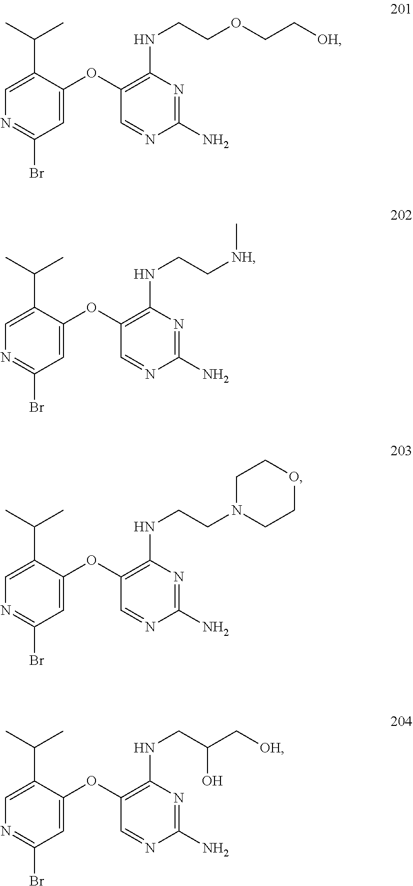 [SMILES: CC(C)c1cnc(Br)cc1Oc1cnc(N)nc1NCCOCCO ; CNCCNc1nc(N)ncc1Oc1cc(Br)ncc1C(C)C ; CC(C)c1cnc(Br)cc1Oc1cnc(N)nc1NCCN1CCOCC1 ; CC(C)c1cnc(Br)cc1Oc1cnc(N)nc1NCC(O)CO]

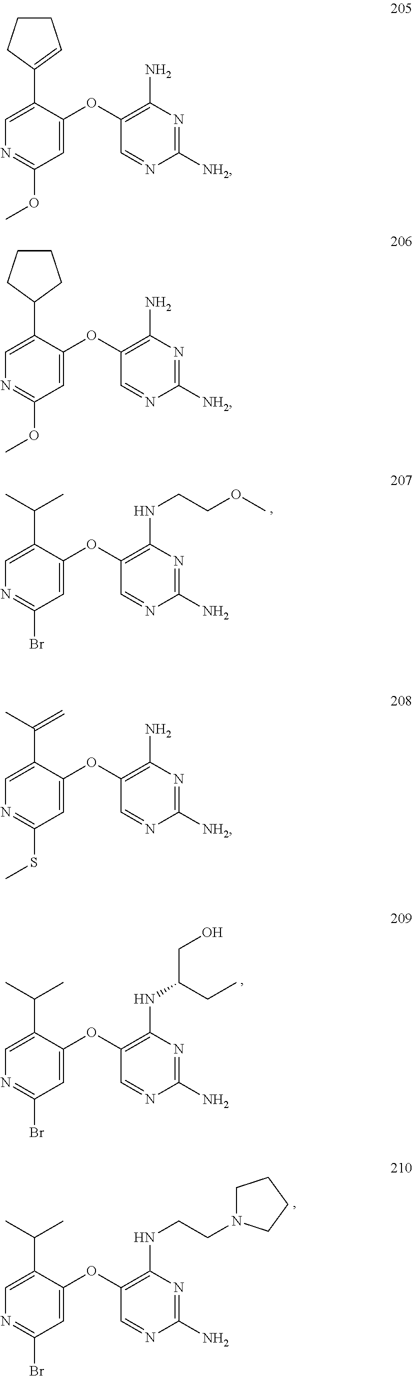

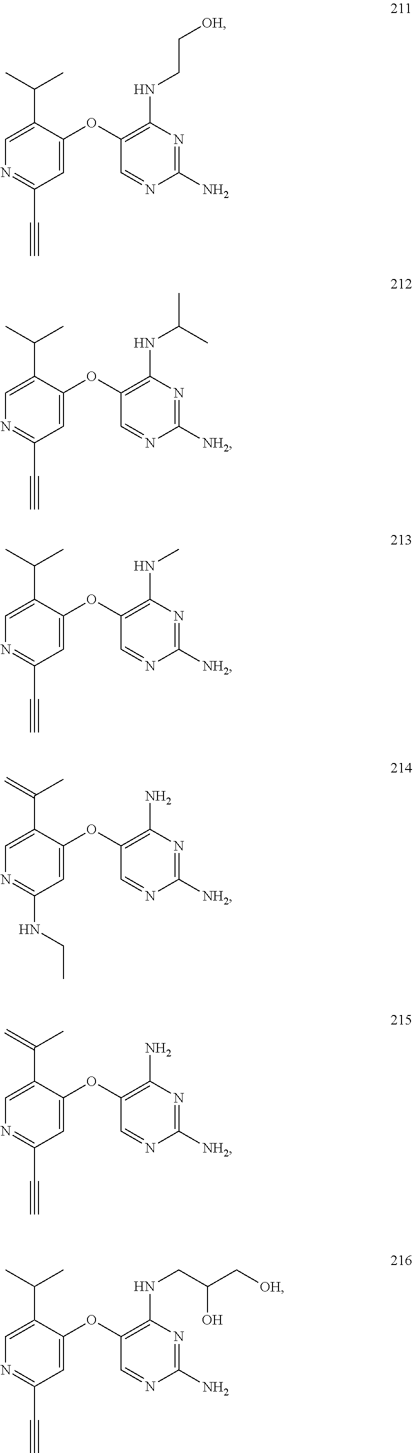 [SMILES: C#Cc1cc(Oc2cnc(N)nc2NCCO)c(C(C)C)cn1 ; C#Cc1cc(Oc2cnc(N)nc2NC(C)C)c(C(C)C)cn1 ; C#Cc1cc(Oc2cnc(N)nc2NC)c(C(C)C)cn1 ; C=C(C)c1cnc(NCC)cc1Oc1cnc(N)nc1N ; C#Cc1cc(Oc2cnc(N)nc2N)c(C(=C)C)cn1 ; C#Cc1cc(Oc2cnc(N)nc2NCC(O)CO)c(C(C)C)cn1]

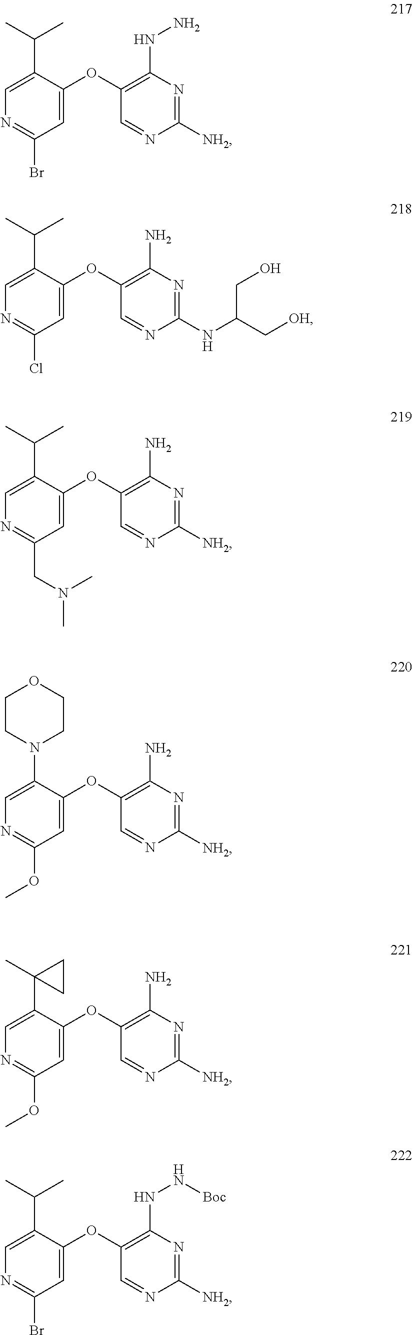 [SMILES: CC(C)c1cnc(Br)cc1Oc1cnc(N)nc1NN ; CC(C)c1cnc(Cl)cc1Oc1cnc(NC(CO)CO)nc1N ; CC(C)c1cnc(CN(C)C)cc1Oc1cnc(N)nc1N ; COc1cc(Oc2cnc(N)nc2N)c(N2CCOCC2)cn1 ; COc1cc(Oc2cnc(N)nc2N)c(C2(C)CC2)cn1 ; CC(C)c1cnc(Br)cc1Oc1cnc(N)nc1NNC(=O)OC(C)(C)C]

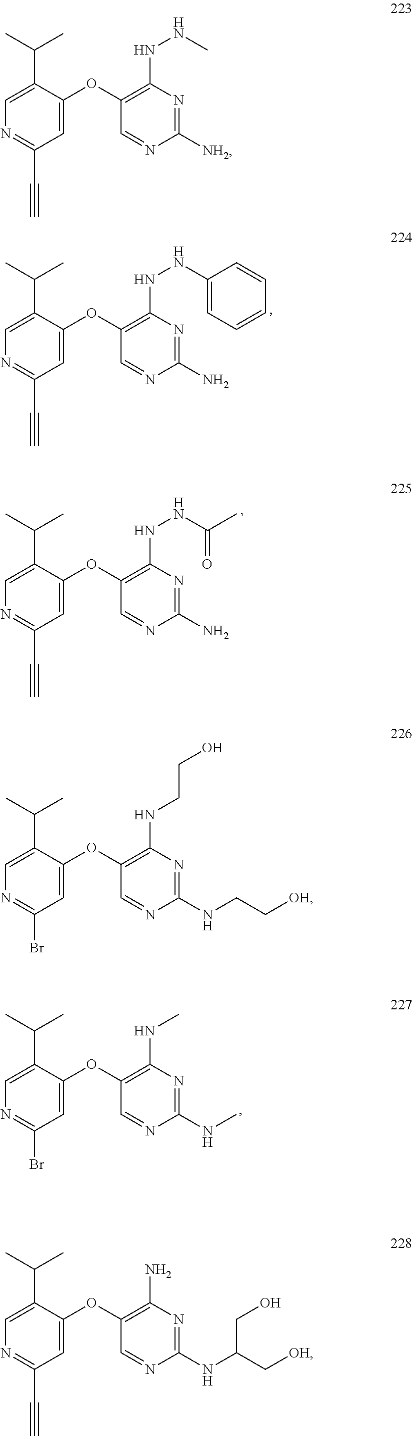 [SMILES: C#Cc1cc(Oc2cnc(N)nc2NNC)c(C(C)C)cn1 ; C#Cc1cc(Oc2cnc(N)nc2NNc2ccccc2)c(C(C)C)cn1 ; C#Cc1cc(Oc2cnc(N)nc2NNC(C)=O)c(C(C)C)cn1 ; CC(C)c1cnc(Br)cc1Oc1cnc(NCCO)nc1NCCO ; CNc1ncc(Oc2cc(Br)ncc2C(C)C)c(NC)n1 ; C#Cc1cc(Oc2cnc(NC(CO)CO)nc2N)c(C(C)C)cn1]

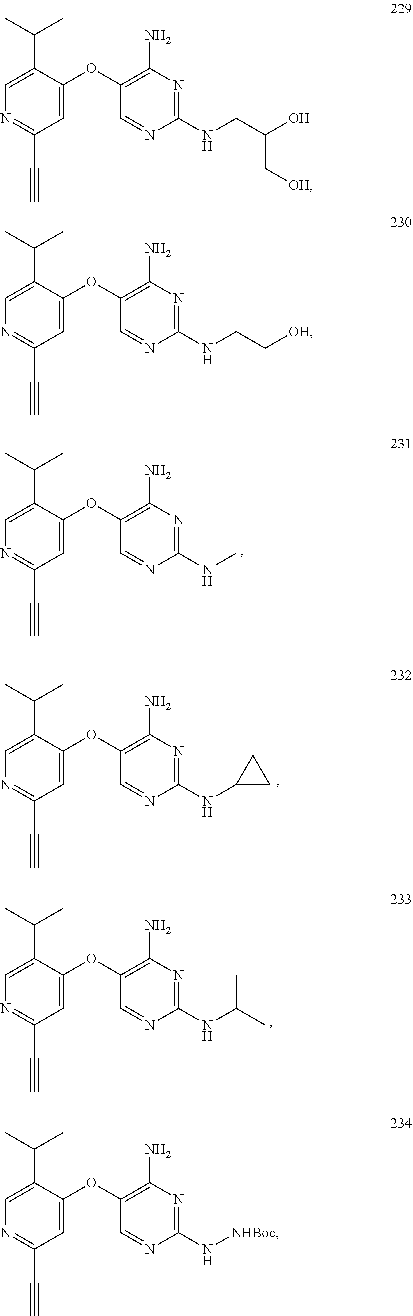

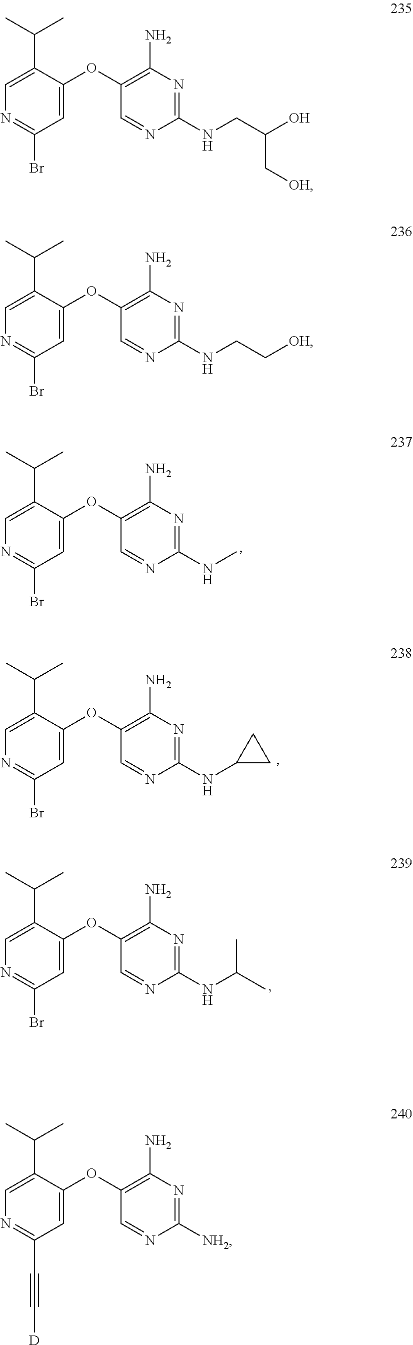 [SMILES: CC(C)c1cnc(Br)cc1Oc1cnc(NCC(O)CO)nc1N ; CC(C)c1cnc(Br)cc1Oc1cnc(NCCO)nc1N ; CNc1ncc(Oc2cc(Br)ncc2C(C)C)c(N)n1 ; CC(C)c1cnc(Br)cc1Oc1cnc(NC2CC2)nc1N ; CC(C)Nc1ncc(Oc2cc(Br)ncc2C(C)C)c(N)n1 ; [2H]C#Cc1cc(Oc2cnc(N)nc2N)c(C(C)C)cn1]

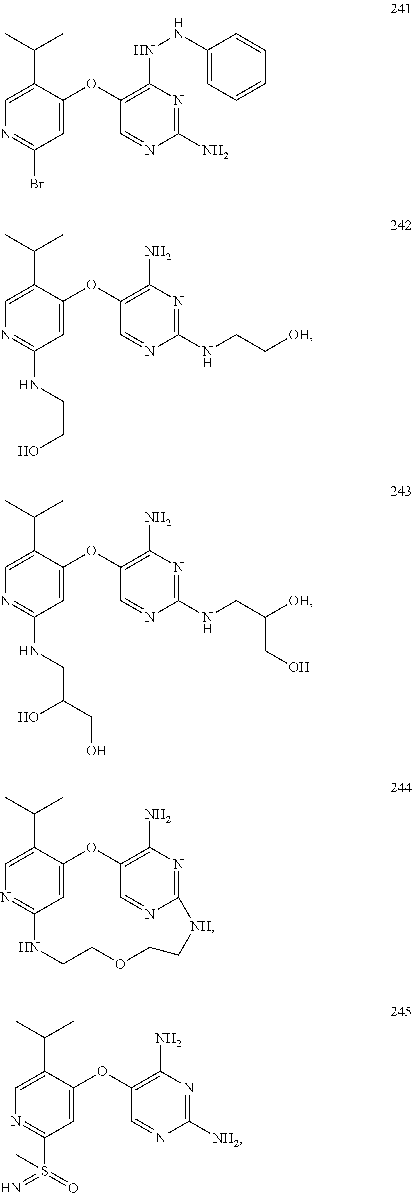 [SMILES: CC(C)c1cnc(Br)cc1Oc1cnc(N)nc1NNc1ccccc1 ; CC(C)c1cnc(NCCO)cc1Oc1cnc(NCCO)nc1N ; CC(C)c1cnc(NCC(O)CO)cc1Oc1cnc(NCC(O)CO)nc1N ; CC(C)c1cnc2cc1Oc1cnc(nc1N)NCCOCCN2 ; CC(C)c1cnc(S(C)(=N)=O)cc1Oc1cnc(N)nc1N]

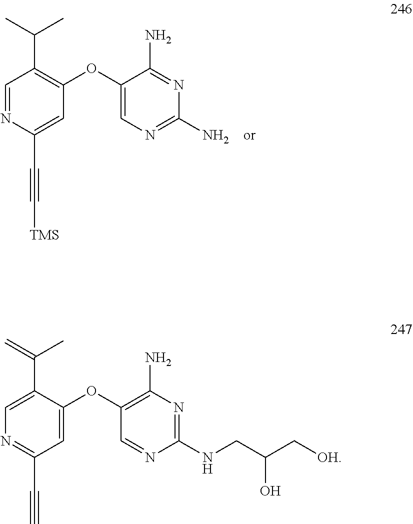 [SMILES: CC(C)c1cnc(C#C[Si](C)(C)C)cc1Oc1cnc(N)nc1N ; C#Cc1cc(Oc2cnc(NCC(O)CO)nc2N)c(C(=C)C)cn1]

In some embodiments, the compound of Formula (I) or a pharmaceutically acceptable salt, ester, stereoisomer, polymorph, solvate, N-oxide, isotopically labeled compound, metabolite or prodmg thereof is administered in an amount of about 0.005 mg/day to about 5000 mg/day, e.g., in an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

In some embodiments, the compound of Formula (I) or a pharmaceutically acceptable salt, ester, stereoisomer, polymorph, solvate, N-oxide, isotopically labeled compound, metabolite or prodmg thereof is administered in an amount of about 1 ng/kg to about 200 mg/kg, about 1 μg/kg to about 100 mg/kg or about 1 mg/kg to about 50 mg/kg per day, e.g., is administered in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg body weight per unit dose.

In some embodiments, the daily dose of the compound of Formula (I) or a pharmaceutically acceptable salt, ester, stereoisomer, polymorph, solvate, N-oxide, isotopically labeled compound, metabolite or prodrug thereof is administered at one time or is administered in two, three or four doses.

In some embodiments, the compound of Formula (I) or a pharmaceutically acceptable salt, ester, stereoisomer, polymorph, solvate, N-oxide, isotopically labeled compound, metabolite or prodmg thereof is administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, at least 50 days, at least half a year, at least 1 year, at least 2 years, at least 3 years, at least 4 years, at least 5 years or more years.

In some embodiments, the compound of Formula (I) or a pharmaceutically acceptable salt, ester, stereoisomer, polymorph, solvate, N-oxide, isotopically labeled compound, metabolite or prodrug thereof is administered for one or more (e.g., 1, 2, 3, 4, 5, 6, 7, 8, 9 or 10) courses of treatment, wherein each course of treatment lasts for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days or at least 50 days; and the interval between every two courses of treatment is 0, 1, 2, 3, 4, 5, 6, 7, 8, 9, 10 days, two weeks, three weeks, or four weeks.

In some embodiments, the compound of Formula (I) or a pharmaceutically acceptable salt, ester, stereoisomer, polymorph, solvate, N-oxide, isotopically labeled compound, metabolite or prodrug thereof is administered through injection (e.g., intravenous, intraarterial, subcutaneous, intraperitoneal, intramuscular injection, including dripping), or transdermal administration, or is administered via oral, buccal, nasal, transmucosal, or topical route, as an ophthalmic formulation, or via inhalation.

In some embodiments, the compound of Formula (I) or a pharmaceutically acceptable salt, ester, stereoisomer, polymorph, solvate, N-oxide, isotopically labeled compound, metabolite or prodrug thereof is administered in a dosage form selected from the group consisting of tablet, capsule, lozenge, hard candy, powder, spray, emulsion, cream, salve, suppository, gel, paste, lotion, injection, nanoformulation, patch, aqueous suspension, injectable solution, elixir, and syrup.

In some embodiments, “cough” is selected from the group consisting of acute cough, sub-acute cough, chronic cough, treatment-resistant cough, treatment-resistant chronic cough, idiopathic chronic cough, post-viral cough, iatrogenic cough, cough associated with post-nasal drip, cough associated with cold, upper respiratory infection, asthma, lung cancer and/or chronic obstructive pulmonary disease (COPD), cough associated with interstitial disease, cough associated with pulmonary fibrosis, cough associated with gastroesophageal reflux disease (GERD), cough associated with smoking or a form of bronchitis, and neuronal hypersensitivity underlying acute, sub-acute or chronic cough; preferably, the cough is selected from the group consisting of chronic cough, treatment-resistant cough, treatment-resistant chronic cough, and cough associated with pulmonary fibrosis.

The method of the present invention can also be applied to various diseases accompanied by cough, for example, various respiratory diseases such as cold (cold syndrome), acute bronchitis, chronic bronchitis, bronchiectasis, pneumonia, pulmonary tuberculosis, silicosis and silicotuberculosis, lung cancer, upper respiratory inflammation (pharyngitis, laryngitis and catarrhal rhinitis), asthmatic bronchitis, bronchial asthma, infantile asthma, (chronic) pulmonary emphysema, pneumoconiosis, pulmonary fibrosis, pulmonary suppuration, pleurisy, tonsillitis, cough hives, pertussis and the like; cough during bronchographic or bronchoscopic examinations or the like.

### EXAMPLE

In order to make the objects and technical solutions of the invention clearer, the invention will be further described below with reference to specific examples. It should be understood that the following examples are only intended for illustrating the invention and are not to be understood as limiting the scope of the invention. Further, specific experimental methods not mentioned in the following examples are carried out in accordance with conventional experimental methods.

Unless otherwise stated, the reagents employed in the following examples were purchased from companies such as Sigma, etc.

Compounds 2 and 66 of the present application were prepared according to the method described in PCT/CN2018/112829 (which is incorporated herein by reference in its entirety).

### Example 1

### Effect on the Guinea Pig Cough Model

In this test, dextromethorphan (purchased from Shanghai Send Pharmaceutical Technology Co., Ltd.) was used as a positive control compound. A clinical control compound having the following structure was employed, and it was prepared according to the method described in WO 2008/040652 A1:

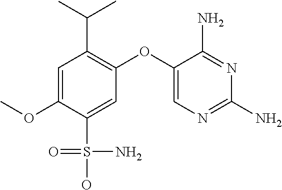

Test animals were 300-350 g Dunkin-Hartley guinea pigs (purchased from Shanghai Jiagan Biotechnology Co., Ltd). After being purchased and adaptively fed for one week, the animals were randomly divided into groups based on body weight, each test group comprising 10-15 test animals The vehicle (a 0.5% CMCNa solution), the test compounds, the positive control compound (dextromethorphan) or the clinical control compound were orally administrated (compounds at test dosages were respectively added to a 0.5% CMCNa solution, uniformly dispersed through thorough ultrasonic treatment; the solution was vortex mixed again before administration to each animal, and the test dosage ranged from 1 mg/kg to 100 mg/kg). Administration was performed 30 minutes to 1 hour before inducing cough.

Method of inducing cough: test animals were put in a body pneumotachograph (Buxco) and allowed to stand for several minutes. After the animals were stable, 250 uM ATP was nebulized for 2 minutes or 0.6 mM histamine was nebulized for 2 minutes. After 3 minutes, 0.5 M citric acid was nebulized for 5 minutes. Starting from the nebulization of citric acid, the number of coughs of the animals in 10 minutes were recorded by employing a small animal pulmonary function detector (Buxco).

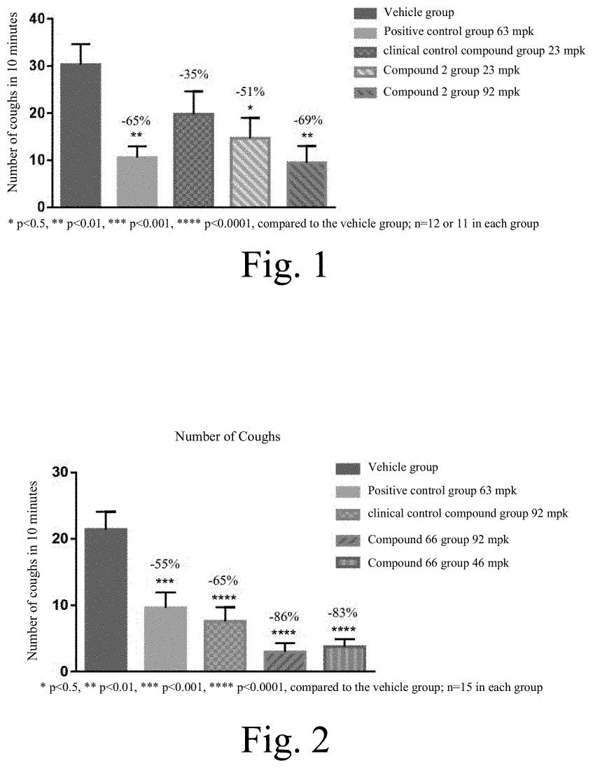

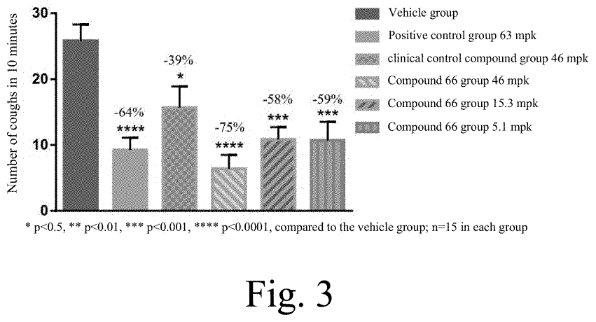

The data in FIG. 1 to FIG. 3 showed that compounds 22 and 66 of the present application had a significant antitussive effect, which was better than that of the clinical control compound.

### Example 2

### Inhibition on the P2X3-Mediated Current in 1321N1 Cell Line Stably Transfected with P2X3

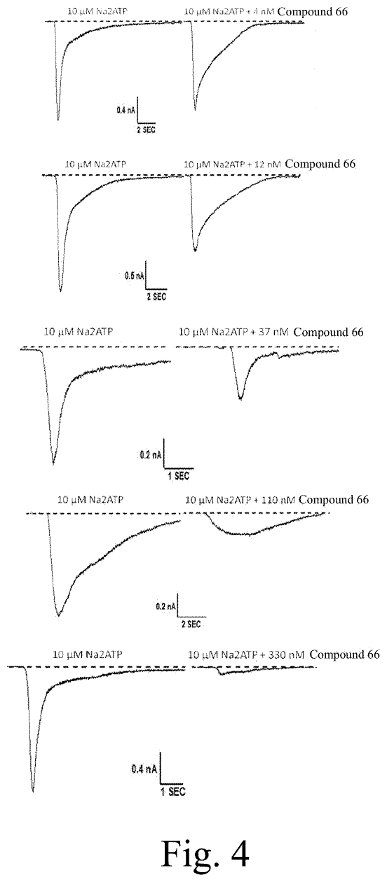

Membrane current was recorded by employing HEKA EPC-10 patch clamp amplifier and PATCHMASTER acquisition system. 1321N1 P2X3 stable transfected cells were transferred to an about 1 ml bath embedded in an inverted microscope platform, and an extracellular fluid (2 mM CaCl₂, 1 mM MgCl₂, 5 mM KCl, 155 mM NaCl, 12 mM glucose and 10 mM HEPES (pH=7.4)) was perfused by using a gravitational perfusion system. The P2X3-mediated current of a single cell was recorded in a whole cell recording mode. After formation of a gigaseal and rapture of the membrane, clamping potential was set at −60 mV. 10 μM Na2ATP was perfused for 5 seconds, and the P2X3-mediated current induced at this point was taken as a control current. The cells were then treated with a solution of compound 66 at a specific concentration (prepared with the extracellular fluid) for 5 minutes. The solution of compound 66 at this concentration and 10 μM Na2ATP were co-applied to induce a cell current (see FIG. 4 for the effect of the compound on the current), and an inhibition rate relative to the control current was calculated according to the following formula:

Inhibition Rate Relative to the Control Current=(1−I₂/I₁)*100%

wherein I₁ represents the control current, and I₂ represents the current after application of compound 66. The concentrations of the test compound 66 included 4, 12, 37, 110 and 330 nM, and at least three cells (n≥3) were tested at each concentration.

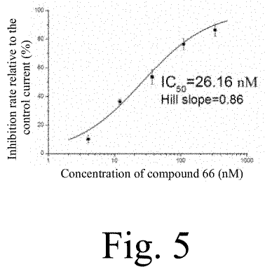

The concentration of compound 66 (as the horizontal axis) was plotted against the inhibition rate relative to the control current (as the vertical axis) (see FIG. 5), and the data were fitted with the Hill equation to obtain that the concentration required for compound 66 to inhibit the P2X3-mediated current induced by 10 μM Na2ATP by 50% (IC₅₀) was 26.16 nM.

### Example 3

### Inhibition on the P2X3-Mediated Current in ex vivo Cultured Rat Dorsal Root Ganglion (DRG)

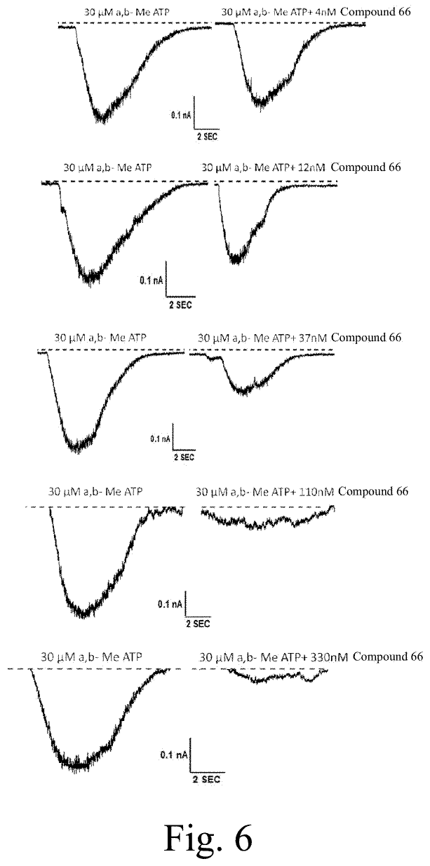

Membrane current was recorded by employing HEKA EPC-10 patch clamp amplifier and PATCHMASTER acquisition system. The primary cells of rat DRG were transferred to an about 1 ml bath embedded in an inverted microscope platform, and an extracellular fluid (2 mM CaCl₂, 1 mM MgCl₂, 5 l mM KCl, 155 mM NaCl, 12 mM glucose and 10 mM HEPES (pH=7.4)) was perfused by using a gravitational perfusion system. The P2X3-mediated current of a single cell was recorded in a whole cell recording mode. After formation of a gigaseal and rapture of the membrane, clamping potential was set at −60 mV. 30 μM a,b-Me ATP (also known as “α,β-meATP”) was perfused for 5 seconds, and the P2X3-mediated current induced at this point was taken as a control current. The cells were then treated with a solution of compound 66 at a specific concentration (prepared with the extracellular fluid) for 5 minutes. The solution of compound 66 at this concentration and 30 μM a,b-Me ATP were co-applied to induce a cell current (see FIG. 6 for the effect of the compound on the current), and an inhibition rate relative to the control current was calculated according to the following formula:

Inhibition Rate Relative to the Control Current=(1−I₂/I₁)*100%

wherein I₁ represents the control current, and I₂ represents the current after application of compound 66. The concentrations of the test compound 66 included 4, 12, 37, 110 and 330 nM, and at least three cells (n≥3) were tested at each concentration.

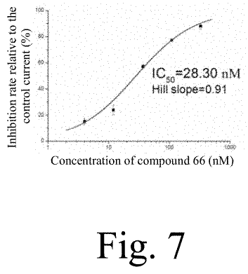

The concentration of compound 66 (as the horizontal axis) was plotted against the inhibition rate relative to the control current (as the vertical axis) (see FIG. 7), and the data were fitted with the Hill equation to obtain that the concentration required for compound 66 to inhibit the P2X3-mediated current induced by 30 μM a,b-Me ATP by 50% (IC₅₀) was 28.30 nM.

Various modifications to the invention in addition to those described herein will become apparent to those skilled in the art from the foregoing description. Such modifications are intended to fall within the scope of the appended claims Each reference, including all patents, applications, journal articles, books and any other disclosure, referred to herein is hereby incorporated by reference in its entirety.

1-11. (canceled)

12. A method for treating, suppressing or alleviating cough or cough impulse, comprising administering to a subject in need thereof a therapeutically effective amount of a compound of Formula (I) or a pharmaceutically acceptable salt, ester, stereoisomer, polymorph, solvate, N-oxide, isotopically labeled compound, metabolite or prodrug thereof: 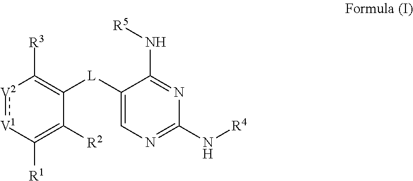
wherein:
L is selected from the group consisting of C(═O), CRR′, NR, O, S, S═O and S(═O)₂;
V¹ is selected from the group consisting of N, 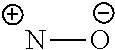
and NR;
V² is selected from the group consisting of CR⁶ and C(═O); [symbol]
represents either a single bond or a double bond, provided that when [symbol] is a single bond, V¹ is NR and V² is C(═O);
R and R′ are each independently selected from the group consisting of H, halogen, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl, and at most 2 ring members in the cyclic hydrocarbyl and heterocyclyl are C(═O);
R¹, R², R³ and R⁶ are each independently selected from the group consisting of H, halogen, —CN, —NO₂, —NH₂, —OH, —SH, —Se—R, —Si(R)₃, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl, C₆₋₁₂ aralkyl, C₁₋₆ haloalkyl, —C(═O)R^{a}, —OC(═O)R^{a}, —C(═O)OR^{a}, —OR^{a}, —SR^{a}, —S(═O)R^{a}, —S(═O)₂R^{a}, —S(═O)₂NR^{a}R^{b}, —S(═O)(═NR)R^{a}, —NR^{a}R^{b}, —C(═O)NR^{a}R^{b}, —C(═S)NR^{a}R^{b}, —C(═NR)NR^{a}R^{b}, —NR^{a}—C(═O)R^{b}, —NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-OR^{a}, —C₁₋₆ alkylene-C(═O)R, —C₁₋₆ alkenylene-OR^{a}, —O—C₁₋₆ alkylene-NR^{a}R^{b} and —P(═O)R^{a}R^{b};
R⁴ and R⁵ are each independently selected from the group consisting of H, —C(═O)OR^{a}, —NR^{a}R^{b}, —NR^{a}—C(═O)R^{b}, —NR^{a}—C(═O)OR^{b}, —C₁₋₆ alkylene-NR^{a}R^{b}, —C₁₋₆ alkylene-OR^{a}, —C₁₋₆ alkylene-O—C₁₋₆ alkylene-OR^{a}, C₁₋₆ alkyl, C₁₋₆ haloalkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl;
alternatively, R¹ and R⁴ together form —NH—(C₁₋₆ alkylene)-L-(C₁₋₆ alkylene)-, preferably —NHCH₂CH₂—O—CH₂CH₂—;
the above alkyl, alkylene, alkenyl, alkynyl, cyclic hydrocarbyl, heterocyclyl, aryl, heteroaryl and aralkyl, at each occurrence, are each optionally substituted with one or more substituents independently selected from the group consisting of halogen, hydroxyl, oxo, amino, cyano, nitro, —Si(R)₃, C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl, C₆₋₁₂ aralkyl, —C(═O)R^{a}, —OC(═O)R^{a}, —C(═O)OR^{a}, —OR^{a}, —SR^{a}, —S(═O)R^{a}, —S(═O)₂R^{a}, —S(═O)₂NR^{a}R^{b}, —NR^{a}R^{b}, —C(═O)NR^{a}R^{b}, —NR^{a}—C(═O)R^{b},
—NR^{a}—C(═O)OR^{b}, —NR^{a}—S(═O)₂—R^{b}, —NR^{a}—C(═O)—NR^{a}R^{b}, —C₁₋₆ alkylene-NR^{a}R^{b}, —C₁₋₆ alkylene-R^{a}, —C₁₋₆ alkenylene-OR^{a} and —O—C₁₋₆ alkylene-NR^{a}R^{b}, the alkyl, cyclic hydrocarbyl, heterocyclyl, aryl, heteroaryl and aralkyl are further optionally substituted with one or more substituents independently selected from the group consisting of halogen, hydroxyl, oxo, amino, cyano, nitro, —NR^{a}R^{b}, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl; and
R^{a} and R^{b}, at each occurrence, are each independently selected from the group consisting of H, —OH, C₁₋₆ alkyl, C₂₋₆ alkenyl, C₂₋₆ alkynyl, saturated or partially unsaturated C₃₋₁₀ cyclic hydrocarbyl, saturated or partially unsaturated 3- to 10-membered heterocyclyl, C₆₋₁₀ aryl, 5- to 14-membered heteroaryl and C₆₋₁₂ aralkyl; alternatively, R^{a} and R^{b} together with the atom to which they are attached form a 3- to 12-membered heterocycle or heteroaromatic ring, the above groups are further optionally substituted with one or more substituents independently selected from the group consisting of halogen, hydroxyl, oxo, amino, cyano, nitro, C₁₋₆ alkyl, —O—C₁₋₆ alkyl, saturated or partially unsaturated C₃₋₆ cyclic hydrocarbyl, saturated or partially unsaturated 3- to 10-membered heterocyclyl, C₆₋₁₀ aryl, 5- to 14-membered heteroaryl and C₆₋₁₂ aralkyl.

13. The method according to claim 12, wherein the compound has the structure of any of the following formulae: 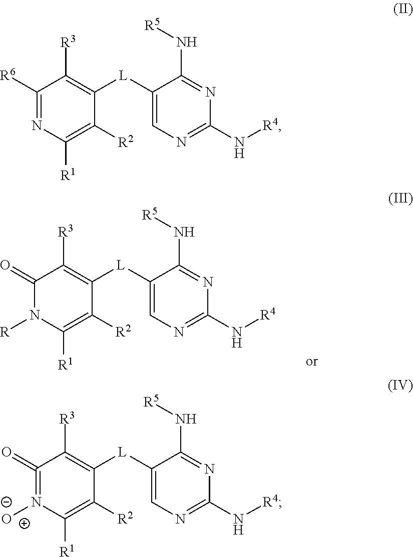
preferably has the structure of any of the following formulae: 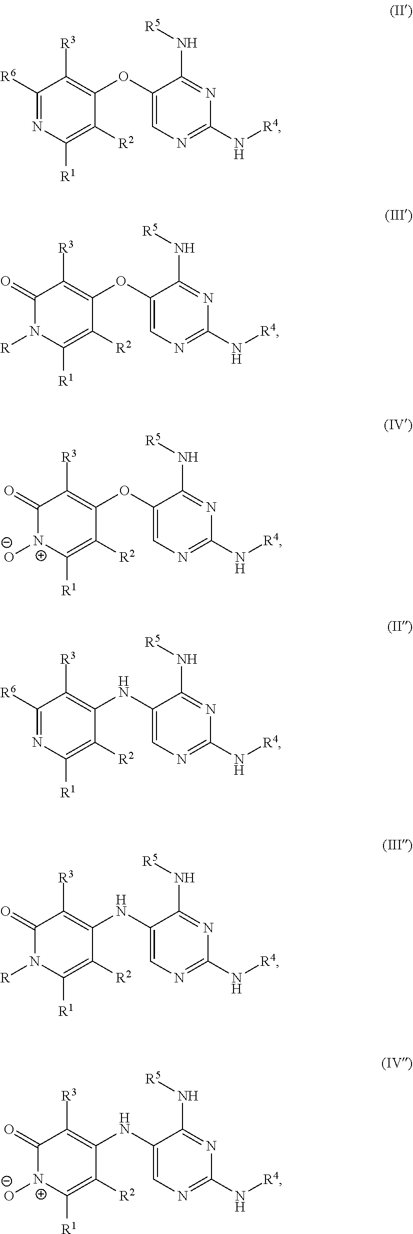 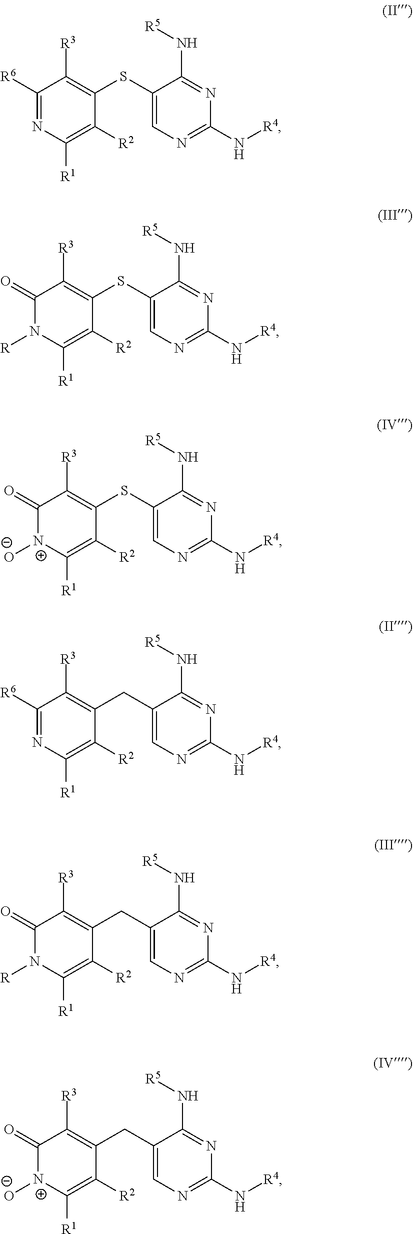
more preferably, the compound of Formula (I) has the structure of any of the following formulae: 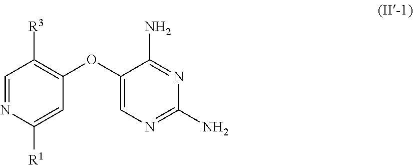
wherein:
R¹ is selected from the group consisting of F, Cl, Br, I and C₂₋₆ alkynyl, preferably Br or ethynyl; and
R³ is C₁₋₆ alkyl, preferably isopropyl.

14. The method according to claim 12, wherein the compound has the following structure:

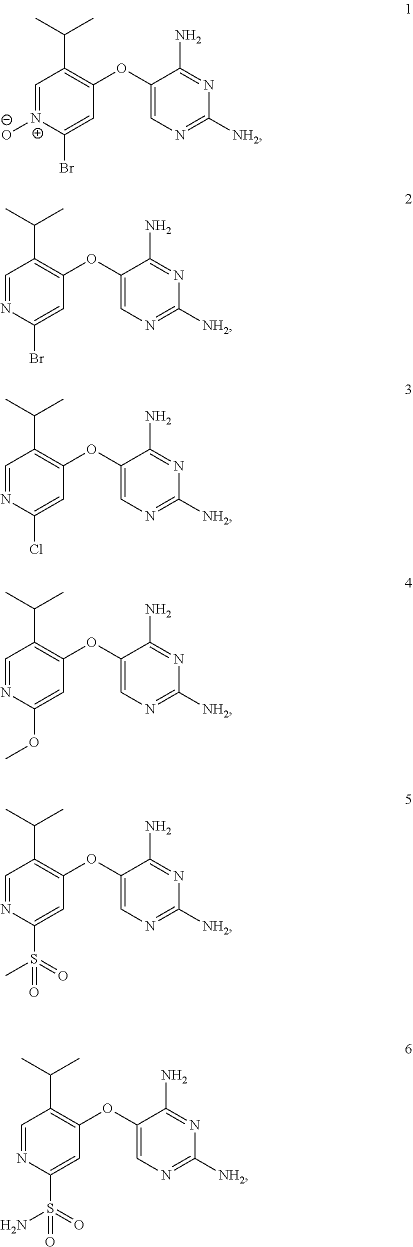

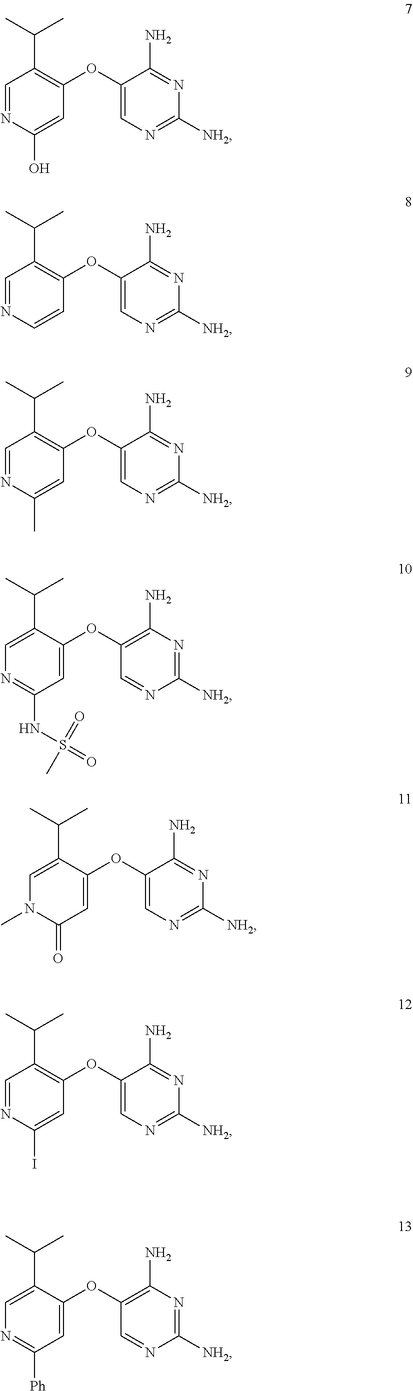

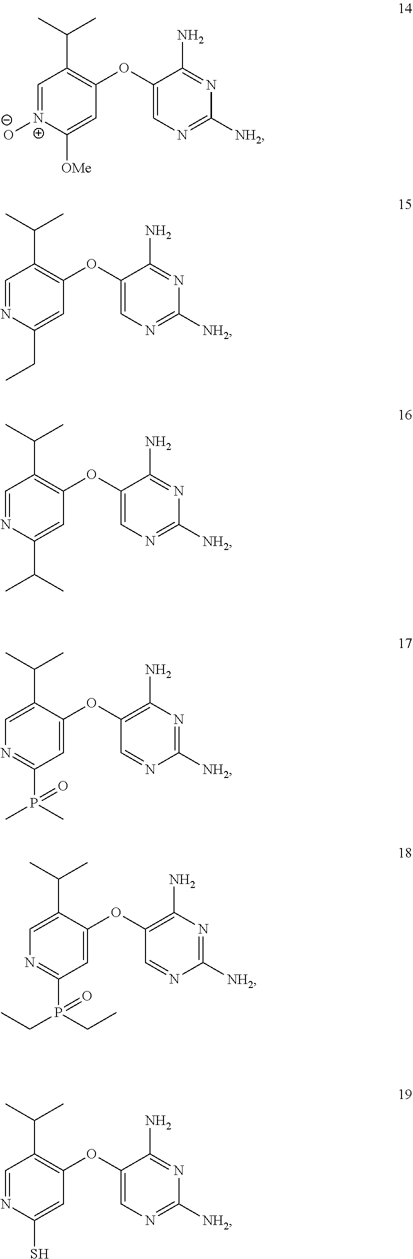

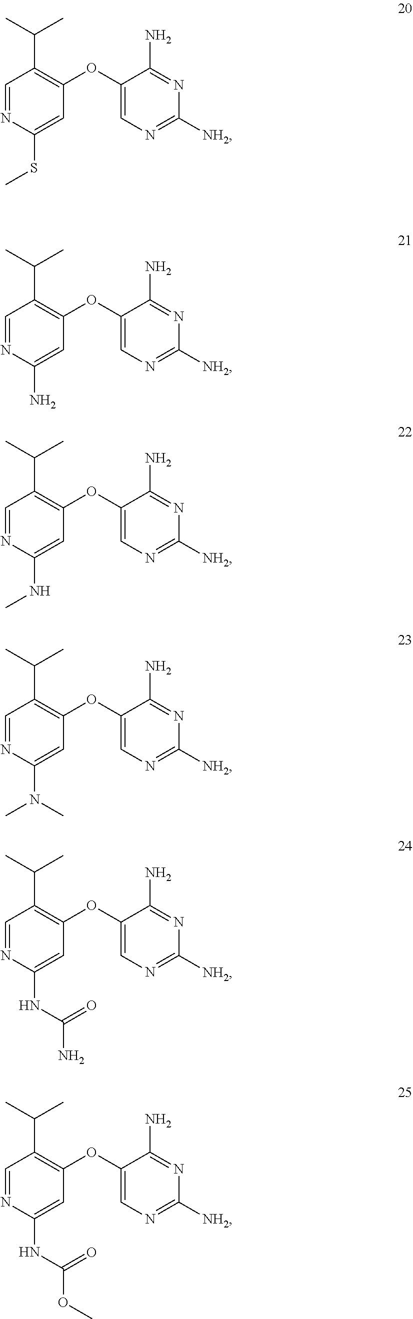

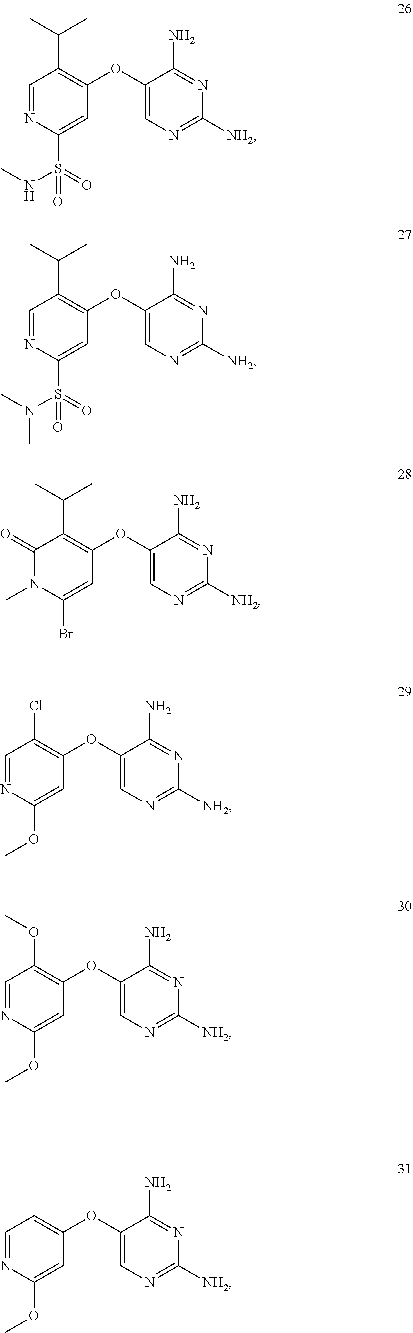

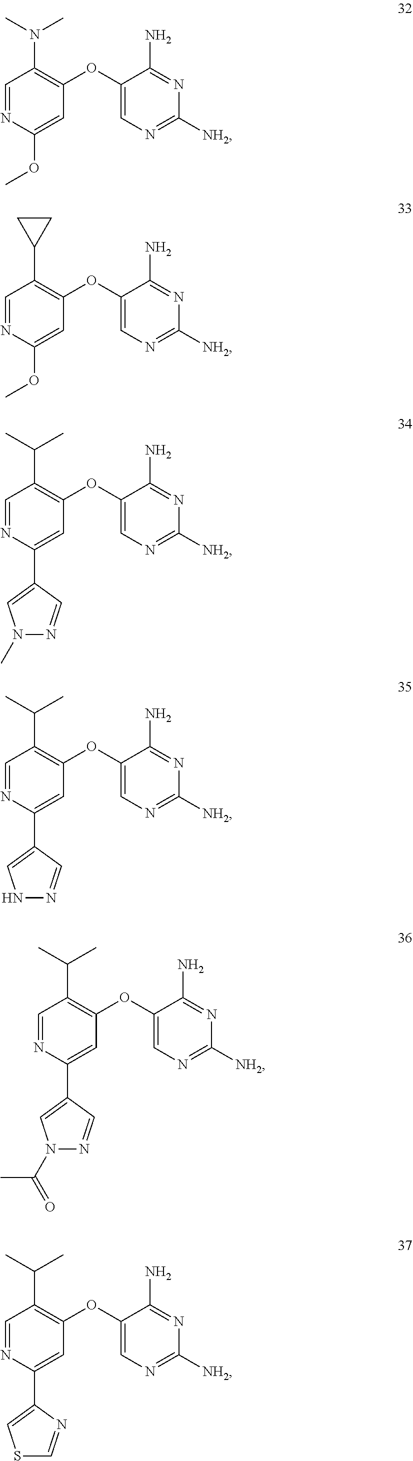

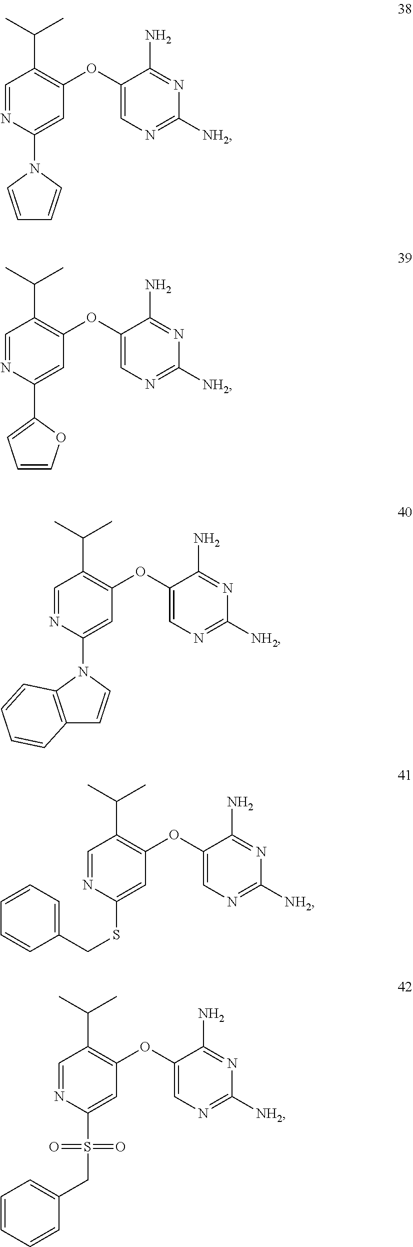 [SMILES: CC(C)c1cnc(-n2cccc2)cc1Oc1cnc(N)nc1N ; CC(C)c1cnc(-c2ccco2)cc1Oc1cnc(N)nc1N ; CC(C)c1cnc(-n2ccc3ccccc32)cc1Oc1cnc(N)nc1N ; CC(C)c1cnc(SCc2ccccc2)cc1Oc1cnc(N)nc1N ; CC(C)c1cnc(S(=O)(=O)Cc2ccccc2)cc1Oc1cnc(N)nc1N]

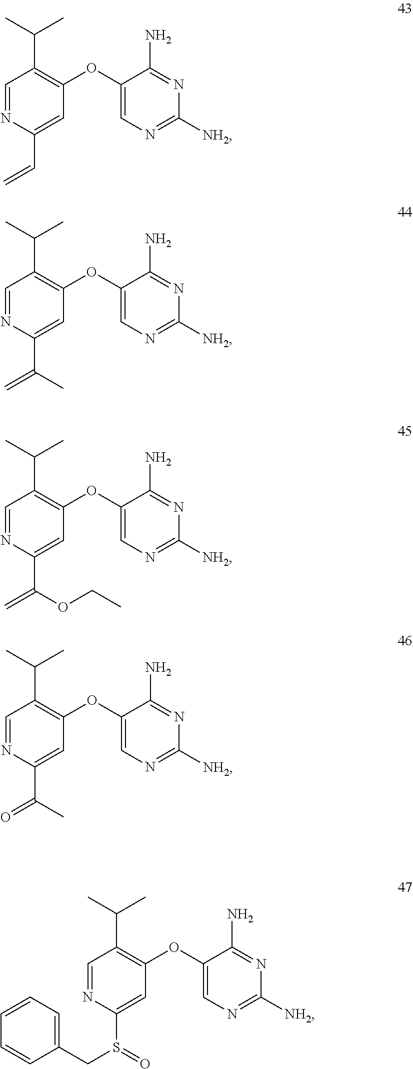

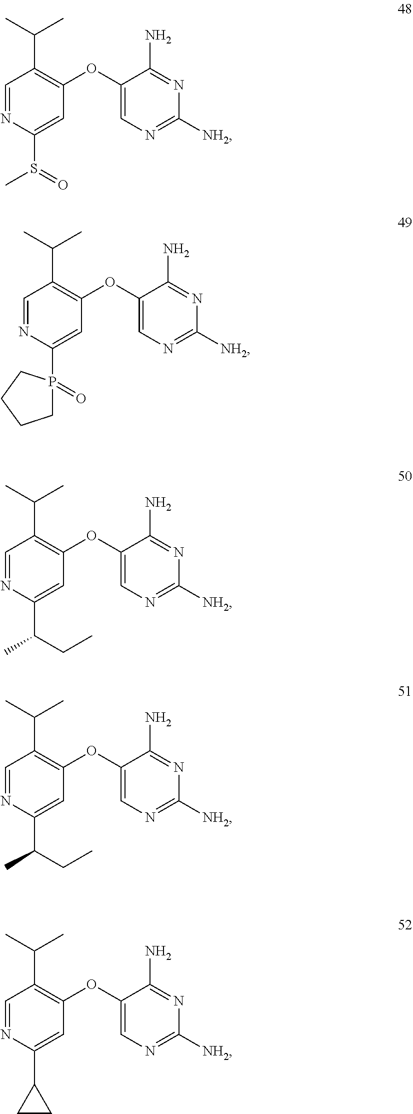 [SMILES: CC(C)c1cnc(S(C)=O)cc1Oc1cnc(N)nc1N ; CC(C)c1cnc(P2(=O)CCCC2)cc1Oc1cnc(N)nc1N ; CC[C@H](C)c1cc(Oc2cnc(N)nc2N)c(C(C)C)cn1 ; CC[C@@H](C)c1cc(Oc2cnc(N)nc2N)c(C(C)C)cn1 ; CC(C)c1cnc(C2CC2)cc1Oc1cnc(N)nc1N]

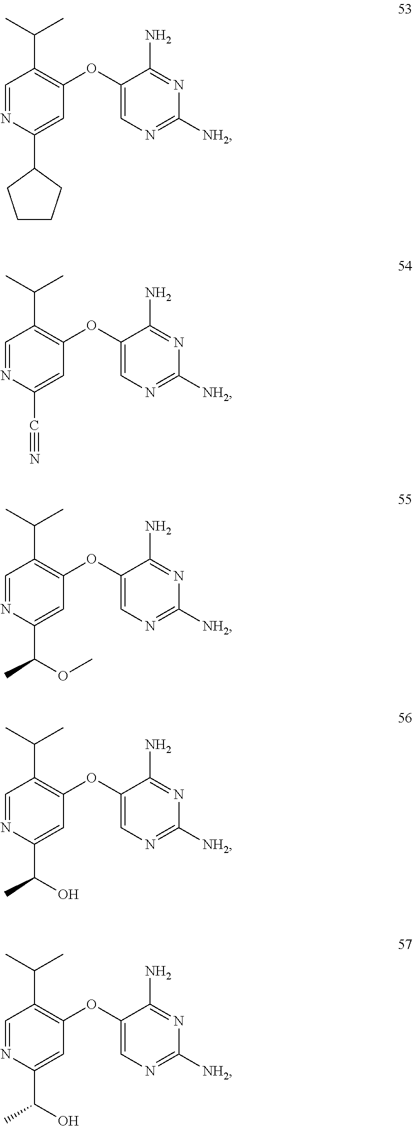 [SMILES: CC(C)c1cnc(C2CCCC2)cc1Oc1cnc(N)nc1N ; CC(C)c1cnc(C#N)cc1Oc1cnc(N)nc1N ; CO[C@@H](C)c1cc(Oc2cnc(N)nc2N)c(C(C)C)cn1 ; CC(C)c1cnc([C@H](C)O)cc1Oc1cnc(N)nc1N ; CC(C)c1cnc([C@@H](C)O)cc1Oc1cnc(N)nc1N]

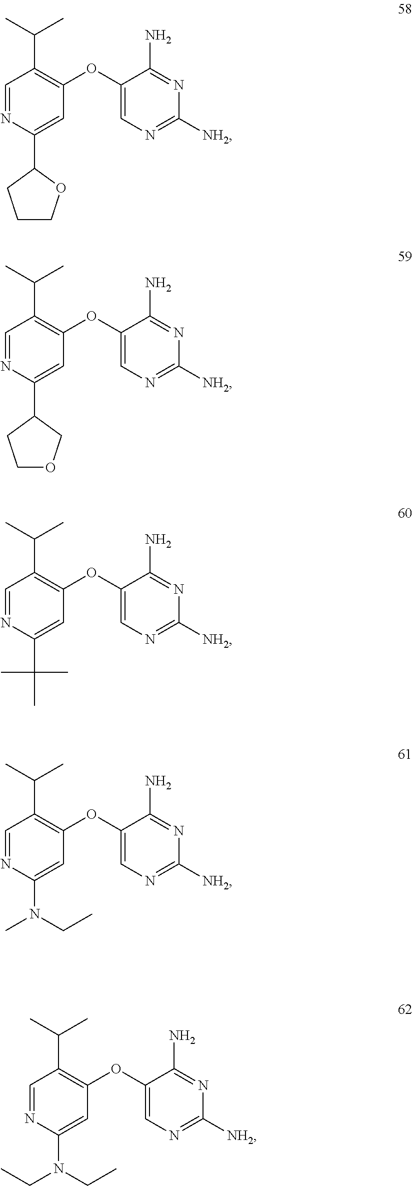 [SMILES: CC(C)c1cnc(C2CCCO2)cc1Oc1cnc(N)nc1N ; CC(C)c1cnc(C2CCOC2)cc1Oc1cnc(N)nc1N ; CC(C)c1cnc(C(C)(C)C)cc1Oc1cnc(N)nc1N ; CCN(C)c1cc(Oc2cnc(N)nc2N)c(C(C)C)cn1 ; CCN(CC)c1cc(Oc2cnc(N)nc2N)c(C(C)C)cn1]

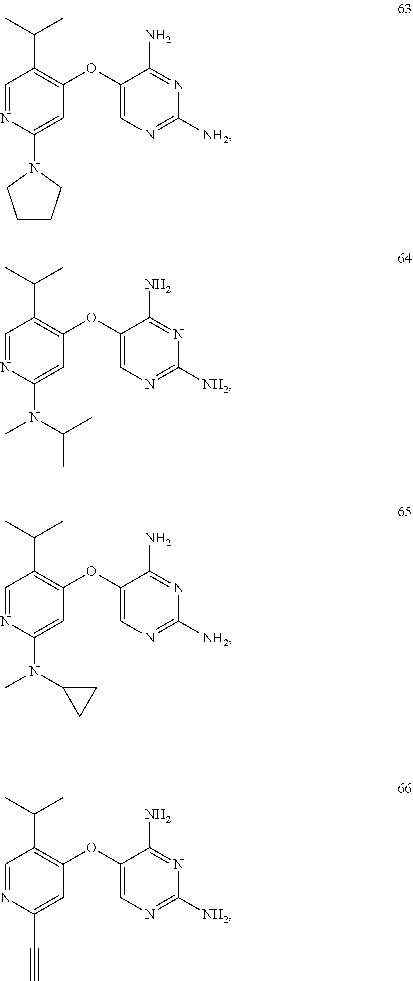 [SMILES: CC(C)c1cnc(N2CCCC2)cc1Oc1cnc(N)nc1N ; CC(C)c1cnc(N(C)C(C)C)cc1Oc1cnc(N)nc1N ; CC(C)c1cnc(N(C)C2CC2)cc1Oc1cnc(N)nc1N ; C#Cc1cc(Oc2cnc(N)nc2N)c(C(C)C)cn1]

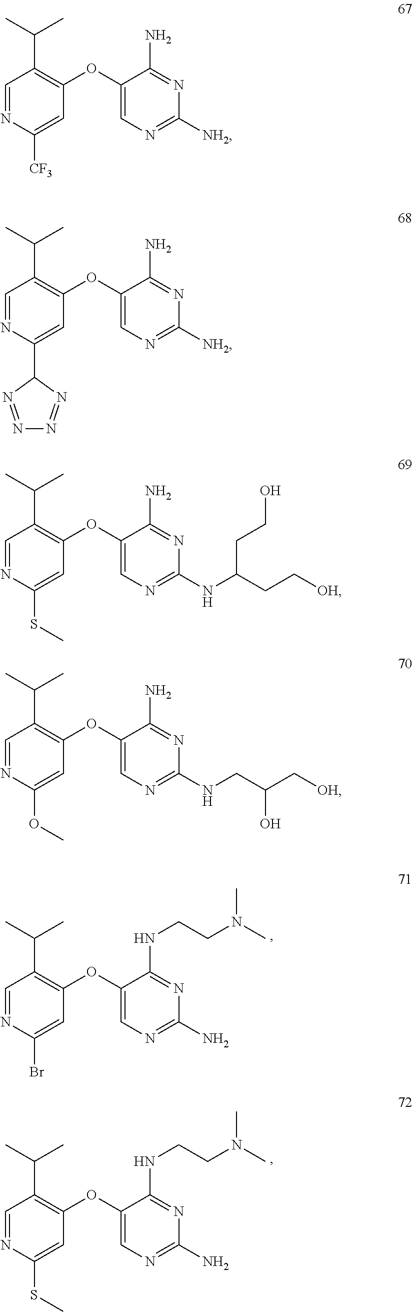

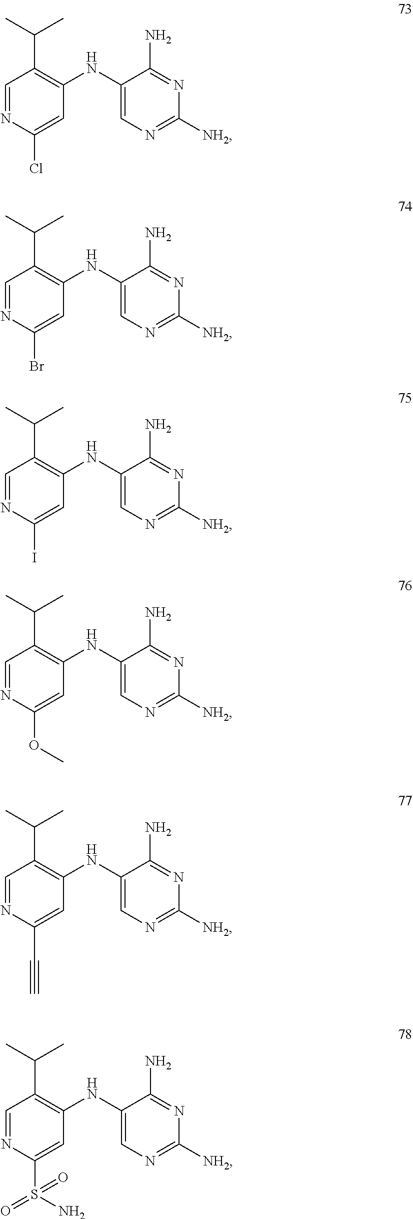 [SMILES: CC(C)c1cnc(Cl)cc1Nc1cnc(N)nc1N ; CC(C)c1cnc(Br)cc1Nc1cnc(N)nc1N ; CC(C)c1cnc(I)cc1Nc1cnc(N)nc1N ; COc1cc(Nc2cnc(N)nc2N)c(C(C)C)cn1 ; C#Cc1cc(Nc2cnc(N)nc2N)c(C(C)C)cn1 ; CC(C)c1cnc(S(N)(=O)=O)cc1Nc1cnc(N)nc1N]

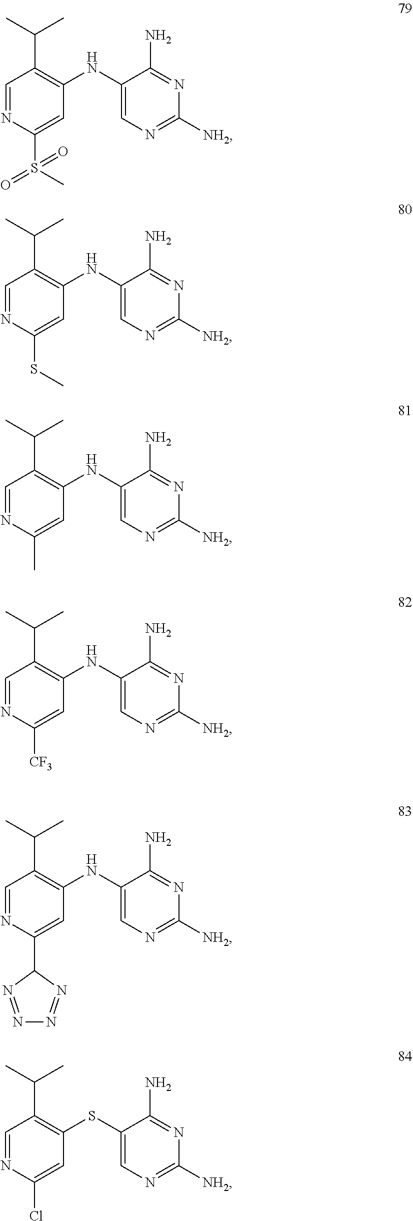

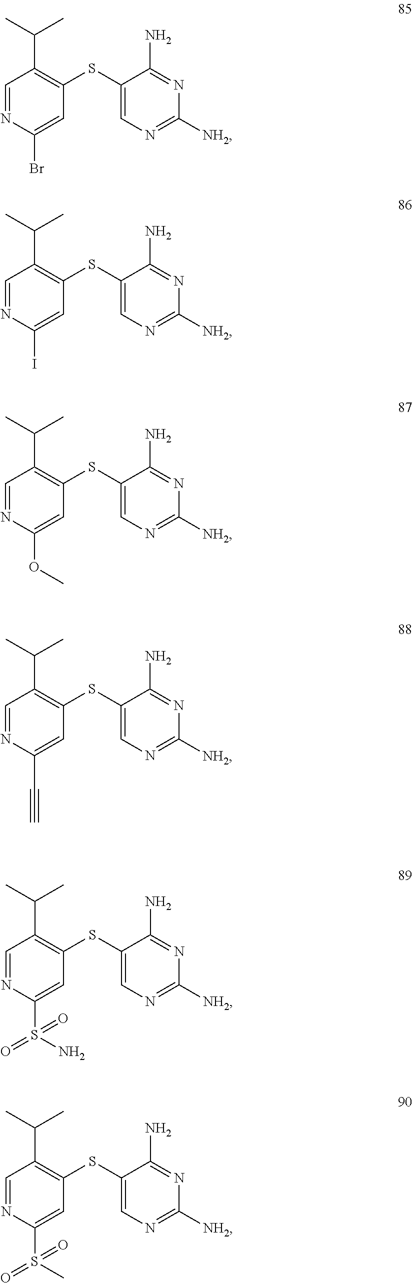 [SMILES: CC(C)c1cnc(Br)cc1Sc1cnc(N)nc1N ; CC(C)c1cnc(I)cc1Sc1cnc(N)nc1N ; COc1cc(Sc2cnc(N)nc2N)c(C(C)C)cn1 ; C#Cc1cc(Sc2cnc(N)nc2N)c(C(C)C)cn1 ; CC(C)c1cnc(S(N)(=O)=O)cc1Sc1cnc(N)nc1N ; CC(C)c1cnc(S(C)(=O)=O)cc1Sc1cnc(N)nc1N]

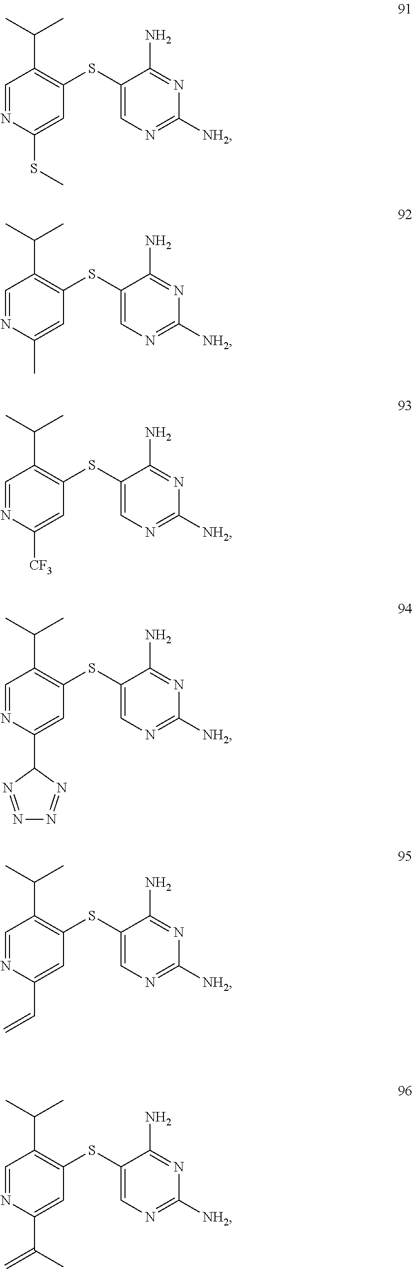

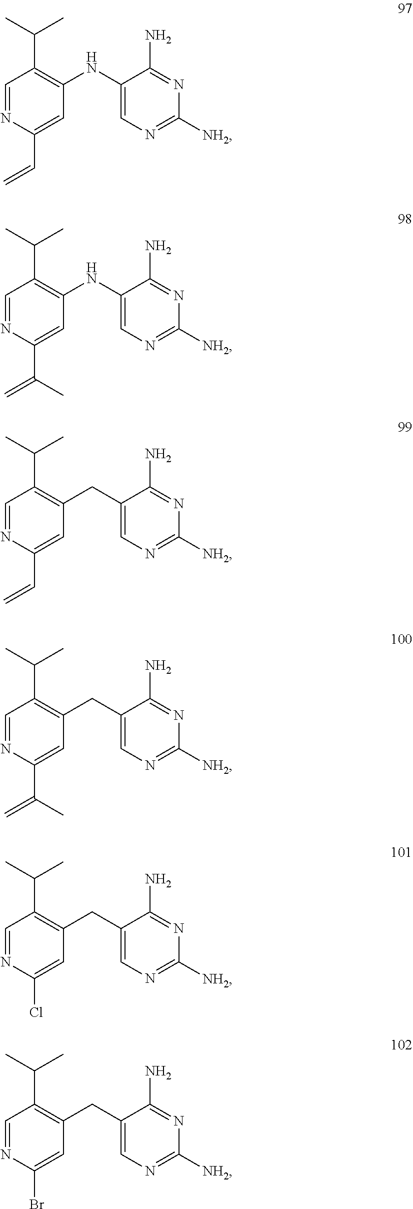

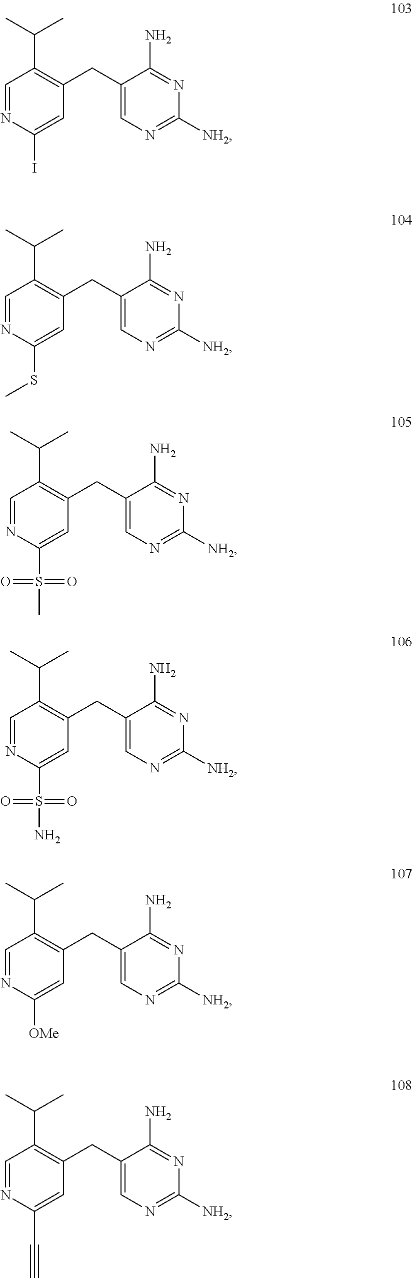

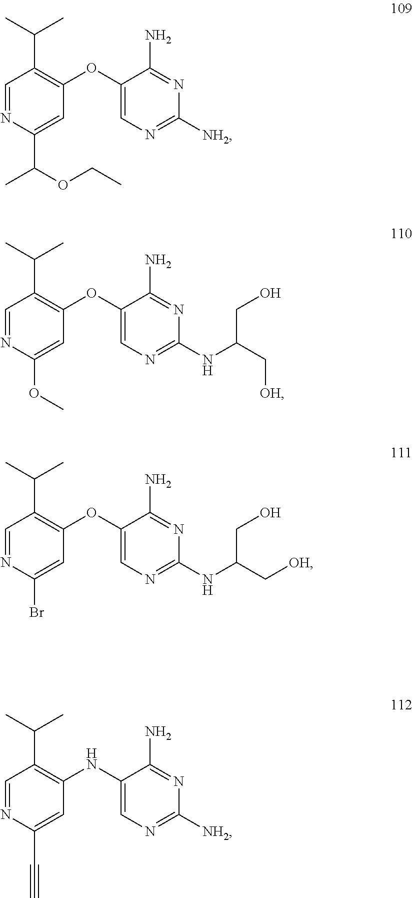 [SMILES: CCOC(C)c1cc(Oc2cnc(N)nc2N)c(C(C)C)cn1 ; COc1cc(Oc2cnc(NC(CO)CO)nc2N)c(C(C)C)cn1 ; CC(C)c1cnc(Br)cc1Oc1cnc(NC(CO)CO)nc1N ; C#Cc1cc(Nc2cnc(N)nc2N)c(C(C)C)cn1]

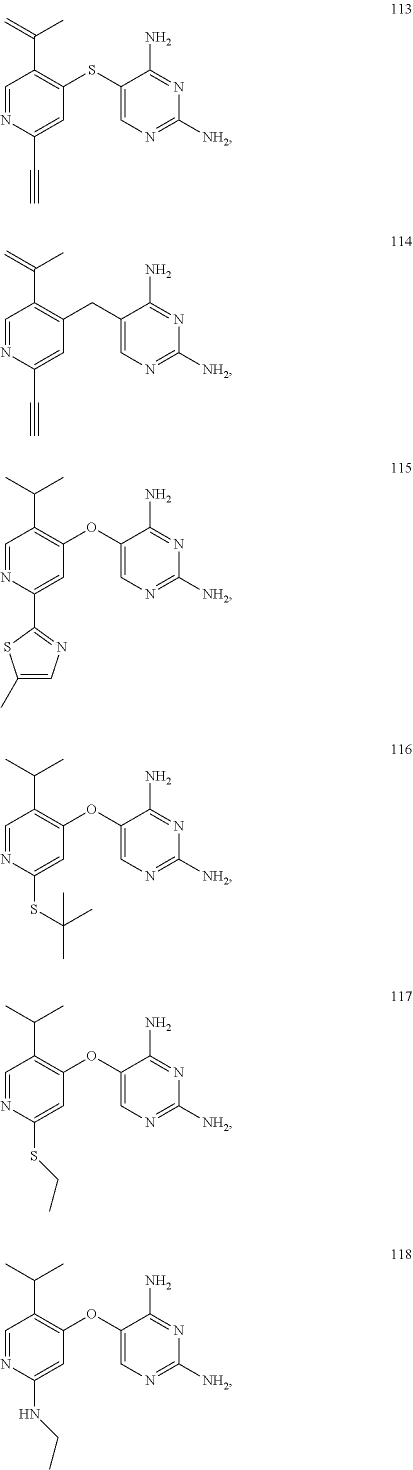

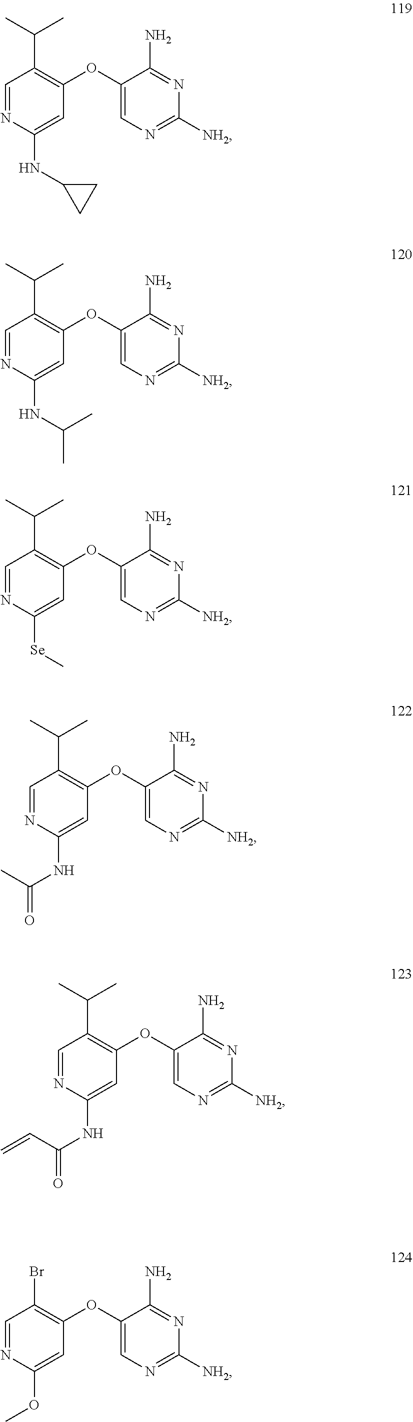

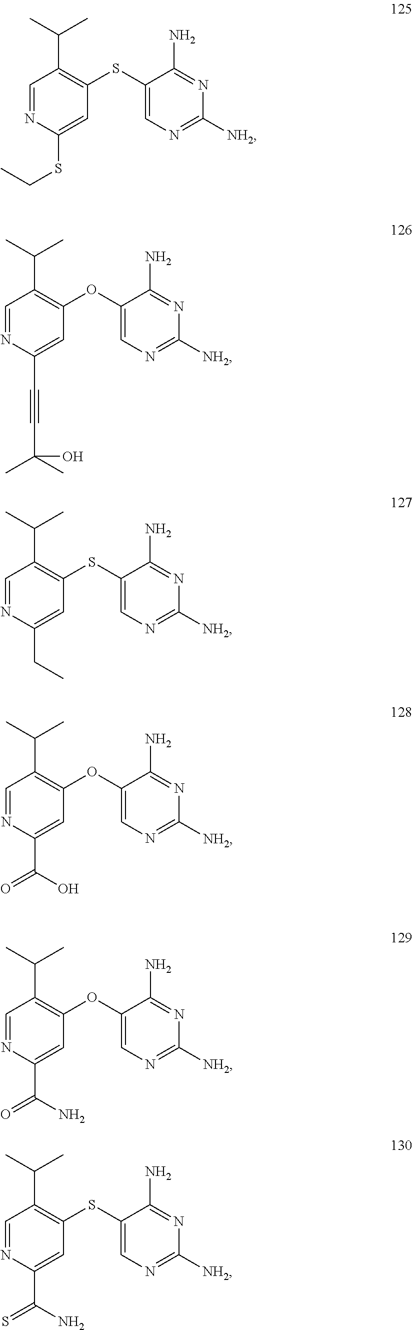

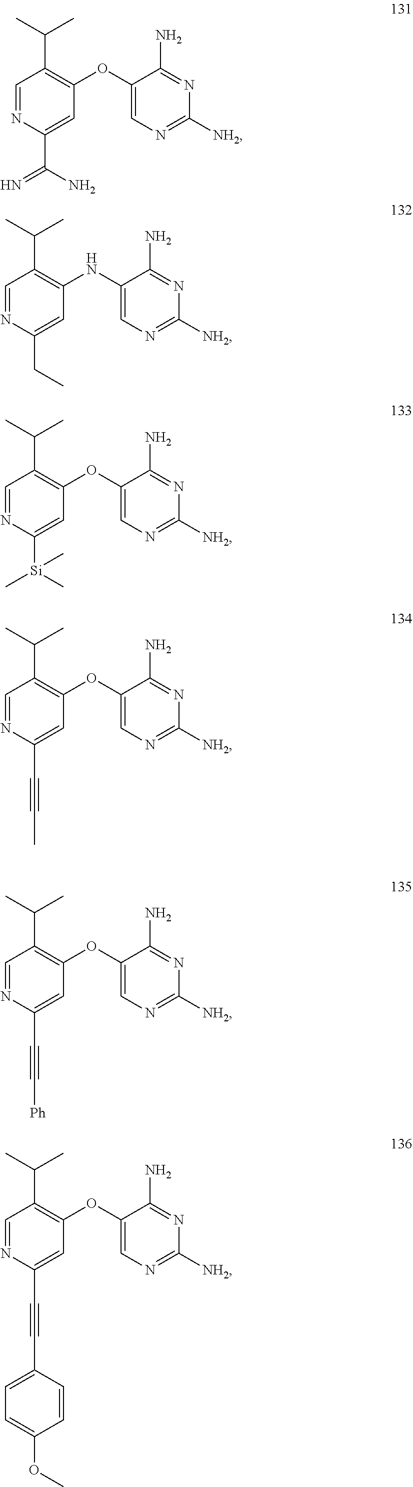

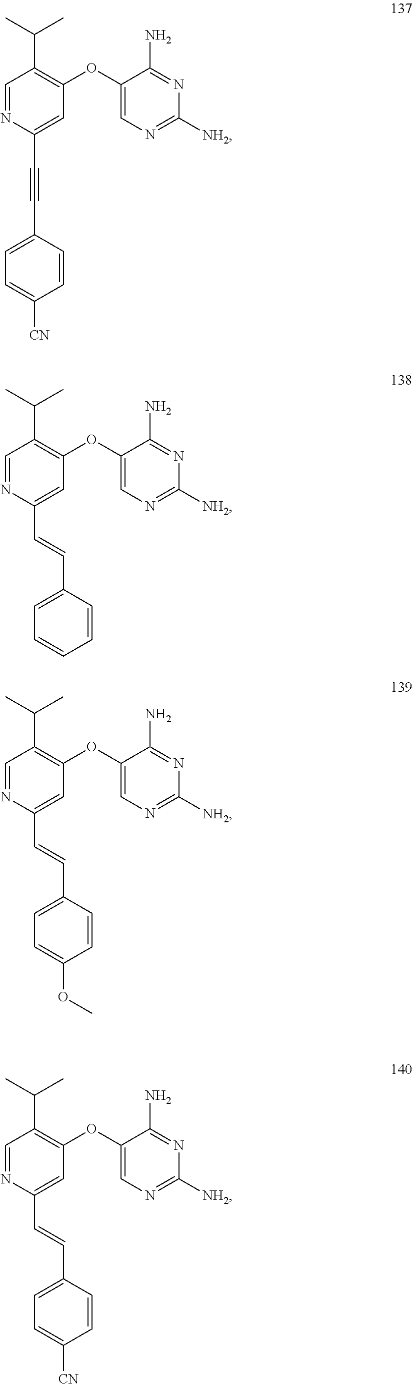

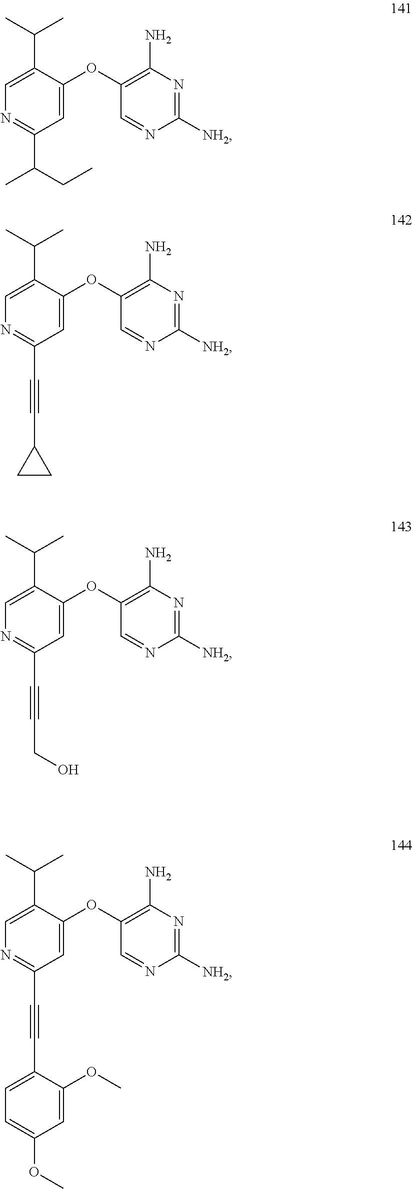

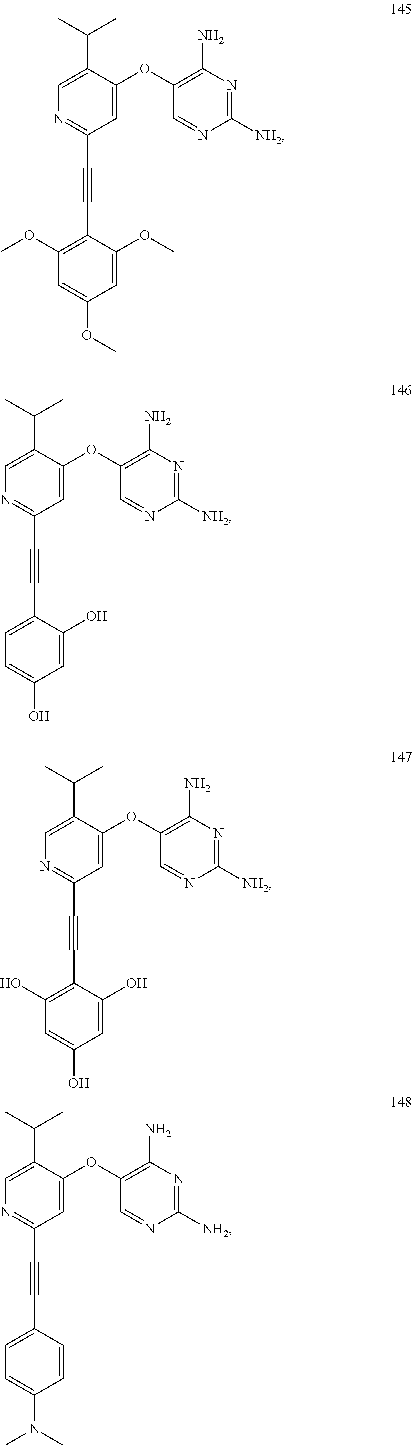

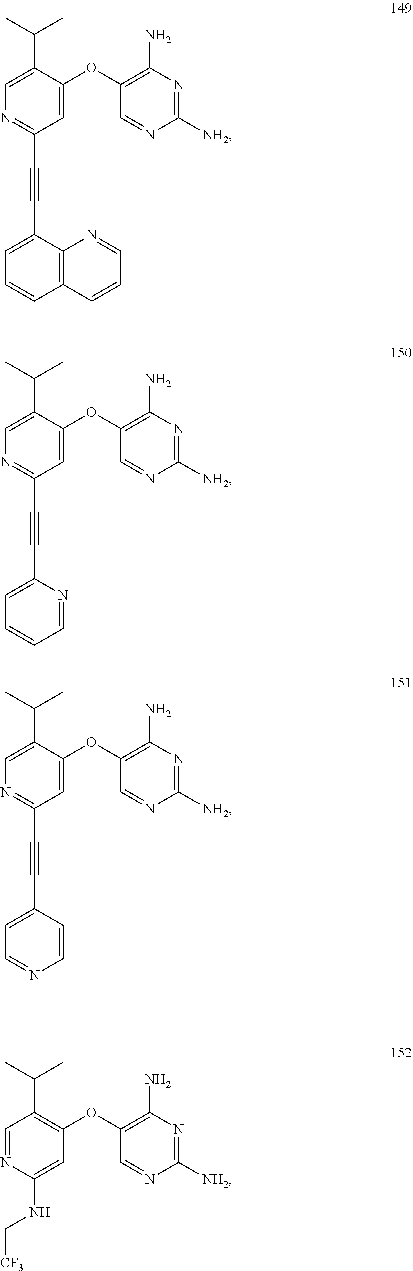 [SMILES: CC(C)c1cnc(C#Cc2cccc3cccnc23)cc1Oc1cnc(N)nc1N ; CC(C)c1cnc(C#Cc2ccccn2)cc1Oc1cnc(N)nc1N ; CC(C)c1cnc(C#Cc2ccncc2)cc1Oc1cnc(N)nc1N ; CC(C)c1cnc(NCC(F)(F)F)cc1Oc1cnc(N)nc1N]

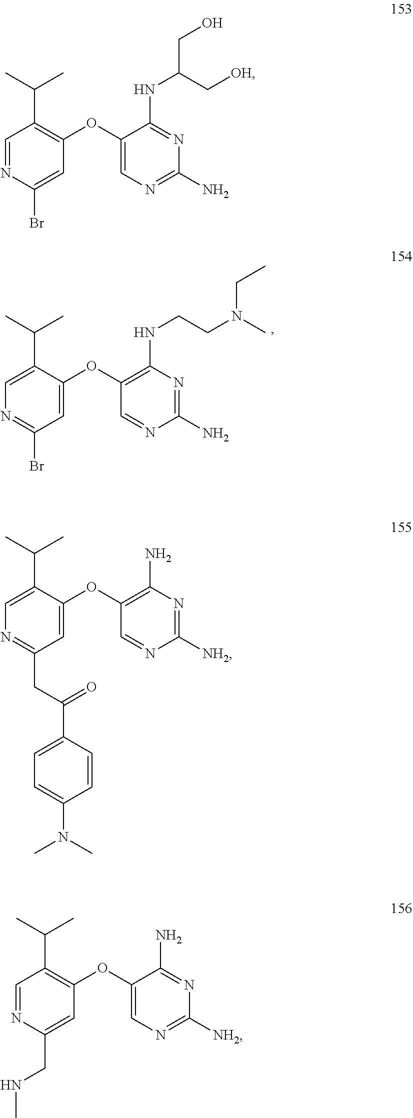 [SMILES: CC(C)c1cnc(Br)cc1Oc1cnc(N)nc1NC(CO)CO ; CCN(C)CCNc1nc(N)ncc1Oc1cc(Br)ncc1C(C)C ; CC(C)c1cnc(CC(=O)c2ccc(N(C)C)cc2)cc1Oc1cnc(N)nc1N ; CNCc1cc(Oc2cnc(N)nc2N)c(C(C)C)cn1]

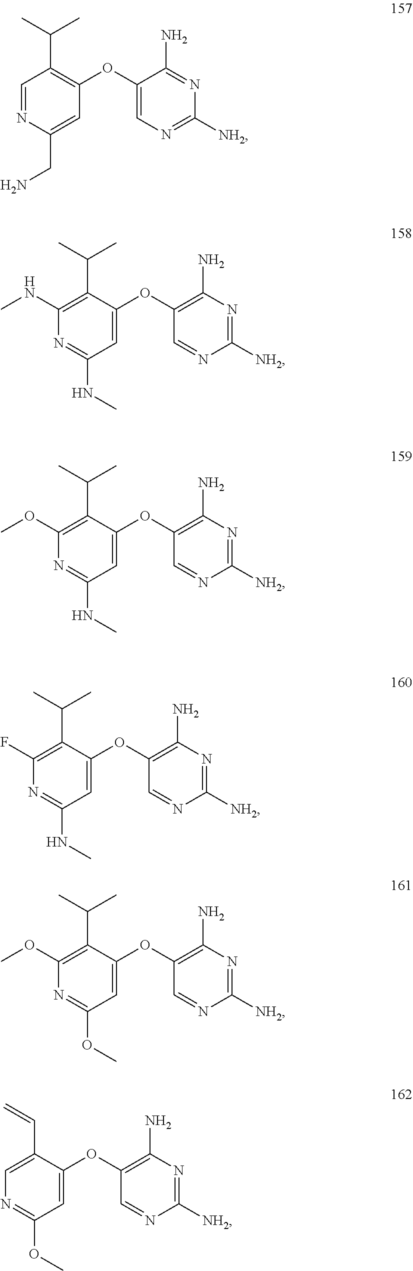

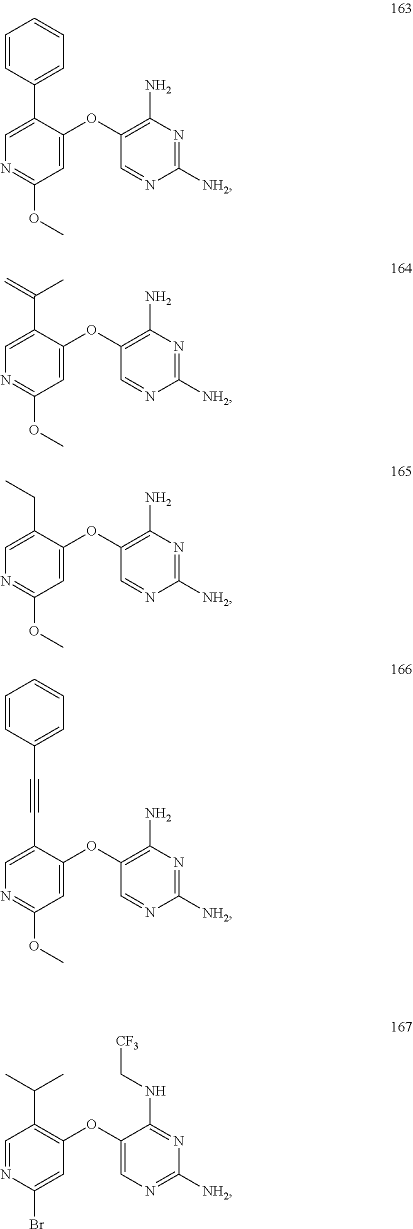

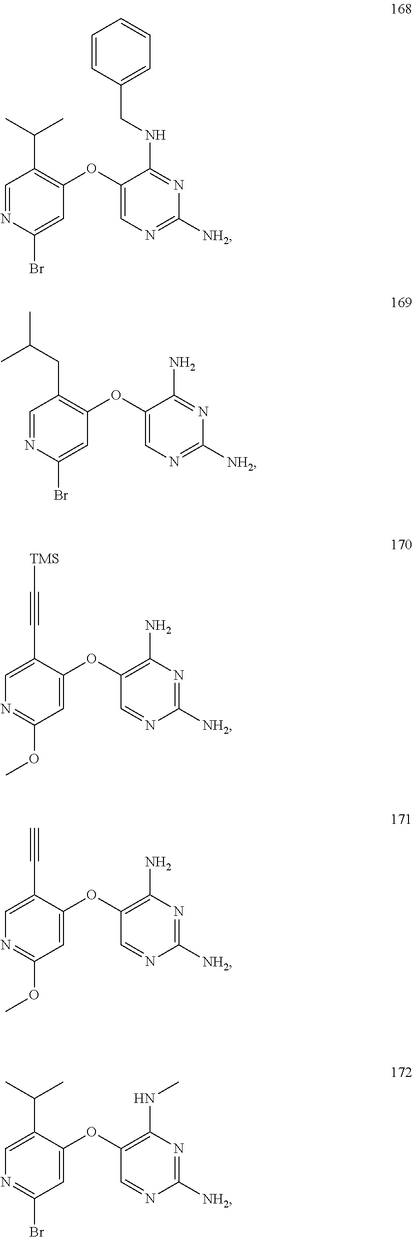

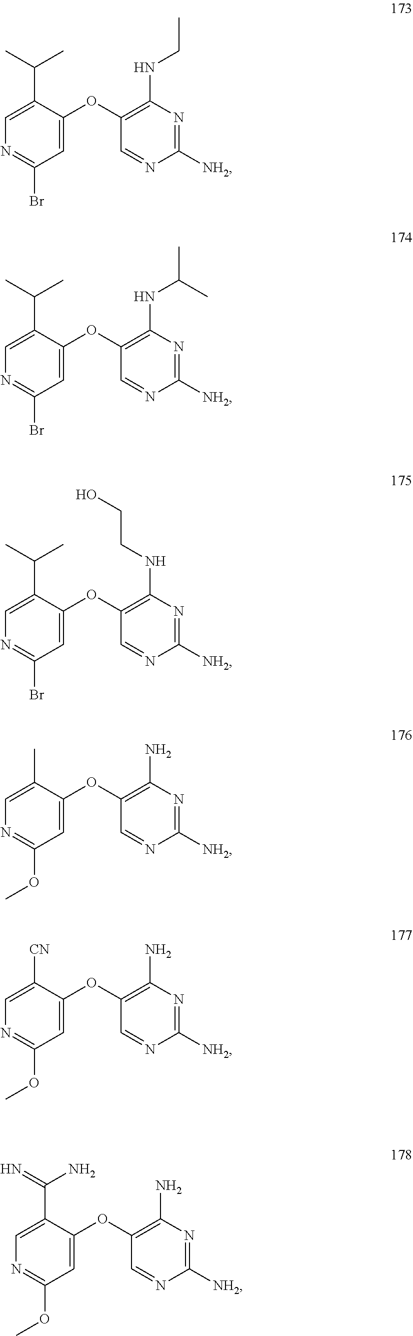 [SMILES: CCNc1nc(N)ncc1Oc1cc(Br)ncc1C(C)C ; CC(C)Nc1nc(N)ncc1Oc1cc(Br)ncc1C(C)C ; CC(C)c1cnc(Br)cc1Oc1cnc(N)nc1NCCO ; COc1cc(Oc2cnc(N)nc2N)c(C)cn1 ; COc1cc(Oc2cnc(N)nc2N)c(C#N)cn1 ; COc1cc(Oc2cnc(N)nc2N)c(C(=N)N)cn1]

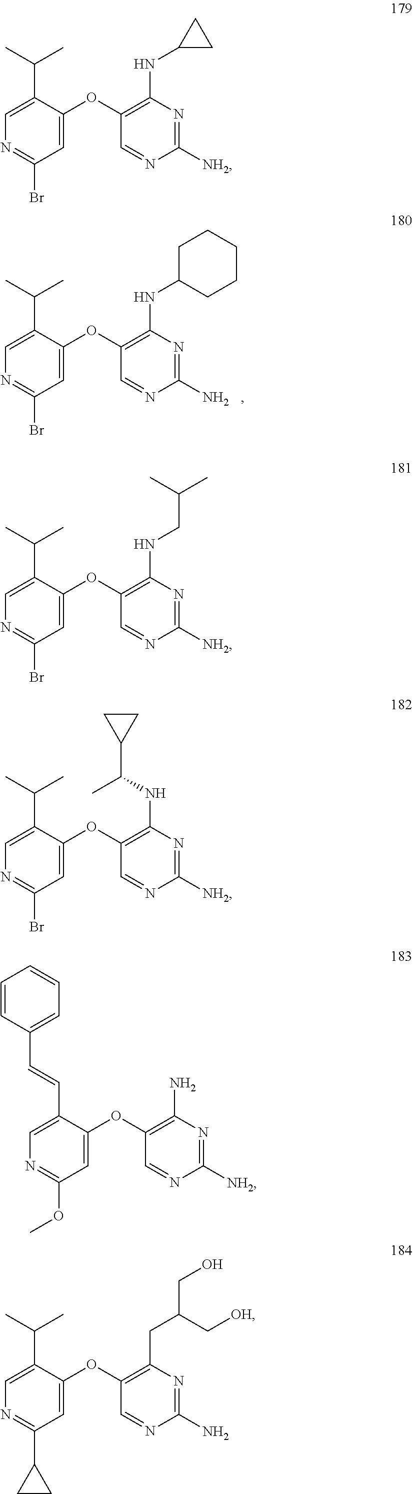

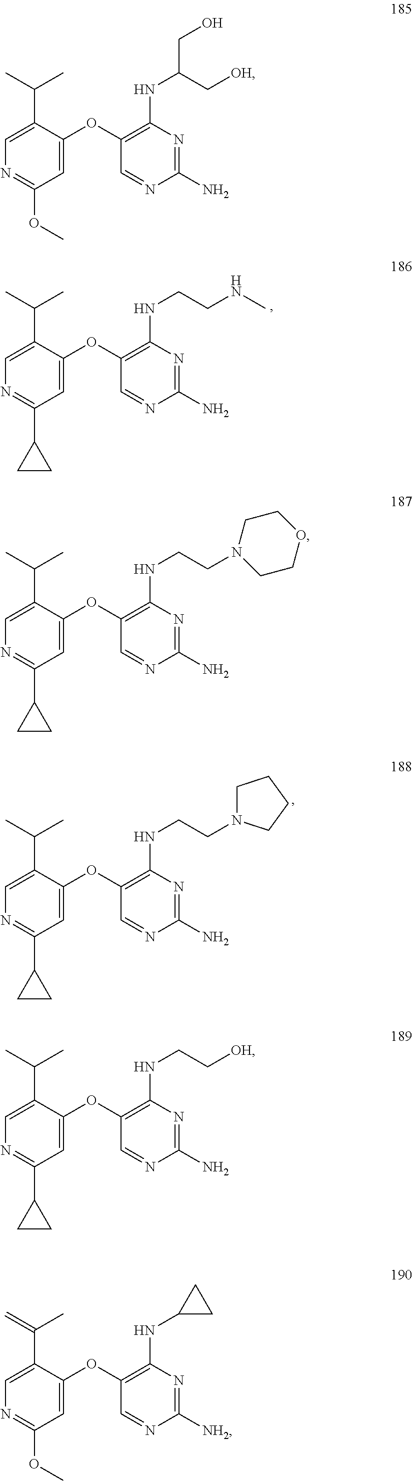 [SMILES: COc1cc(Oc2cnc(N)nc2NC(CO)CO)c(C(C)C)cn1 ; CNCCNc1nc(N)ncc1Oc1cc(C2CC2)ncc1C(C)C ; CC(C)c1cnc(C2CC2)cc1Oc1cnc(N)nc1NCCN1CCOCC1 ; CC(C)c1cnc(C2CC2)cc1Oc1cnc(N)nc1NCCN1CCCC1 ; CC(C)c1cnc(C2CC2)cc1Oc1cnc(N)nc1NCCO ; C=C(C)c1cnc(OC)cc1Oc1cnc(N)nc1NC1CC1]

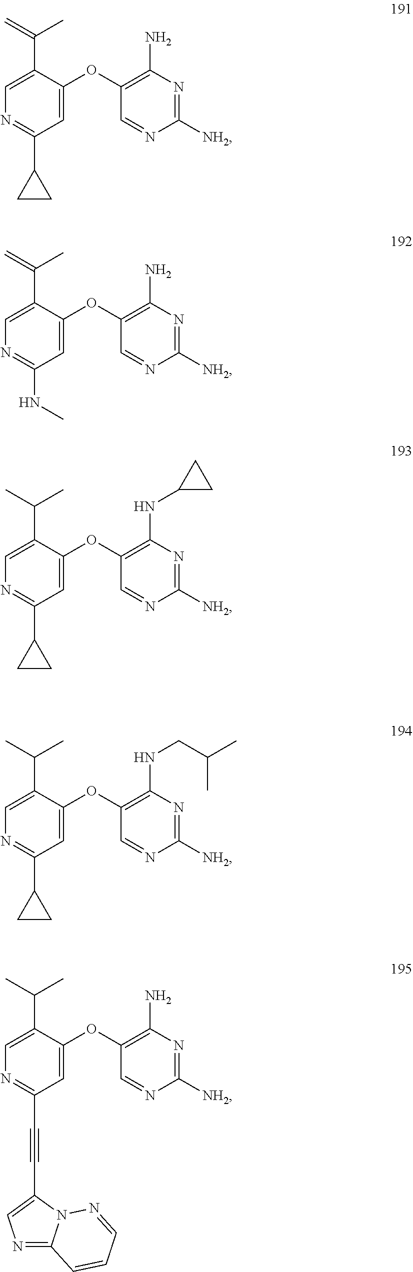 [SMILES: C=C(C)c1cnc(C2CC2)cc1Oc1cnc(N)nc1N ; C=C(C)c1cnc(NC)cc1Oc1cnc(N)nc1N ; CC(C)c1cnc(C2CC2)cc1Oc1cnc(N)nc1NC1CC1 ; CC(C)CNc1nc(N)ncc1Oc1cc(C2CC2)ncc1C(C)C ; CC(C)c1cnc(C#Cc2cnc3cccnn23)cc1Oc1cnc(N)nc1N]

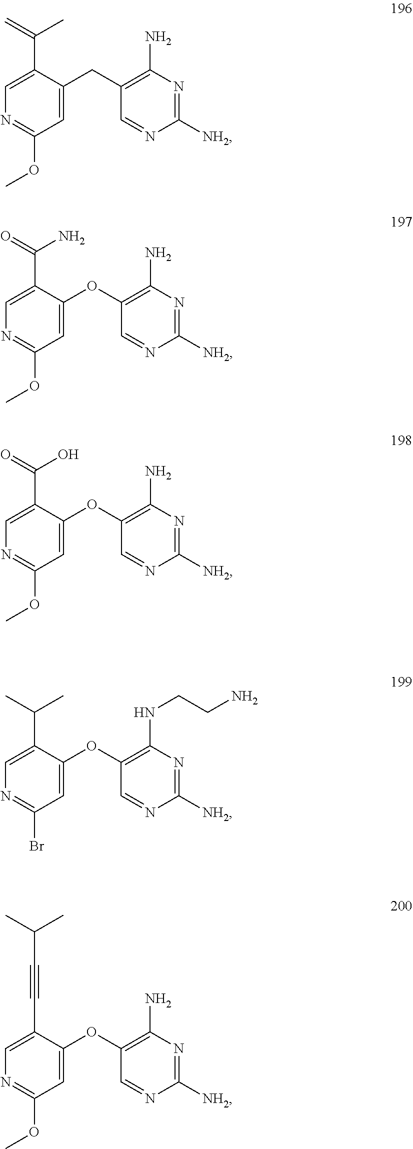 [SMILES: C=C(C)c1cnc(OC)cc1Cc1cnc(N)nc1N ; COc1cc(Oc2cnc(N)nc2N)c(C(N)=O)cn1 ; COc1cc(Oc2cnc(N)nc2N)c(C(=O)O)cn1 ; CC(C)c1cnc(Br)cc1Oc1cnc(N)nc1NCCN ; COc1cc(Oc2cnc(N)nc2N)c(C#CC(C)C)cn1]

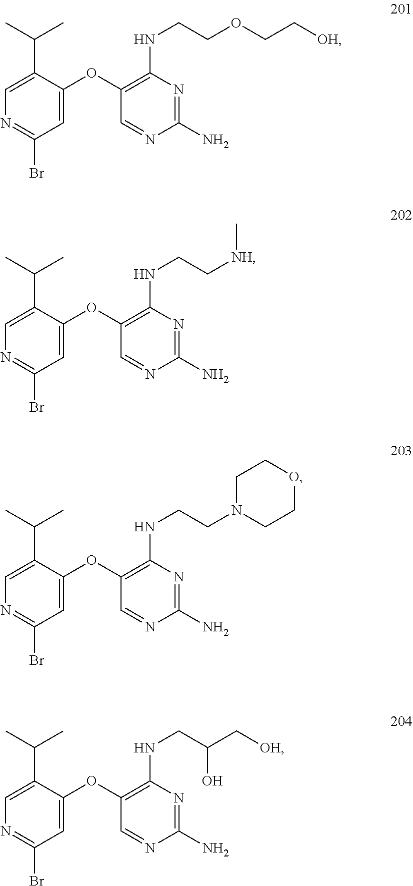 [SMILES: CC(C)c1cnc(Br)cc1Oc1cnc(N)nc1NCCOCCO ; CNCCNc1nc(N)ncc1Oc1cc(Br)ncc1C(C)C ; CC(C)c1cnc(Br)cc1Oc1cnc(N)nc1NCCN1CCOCC1 ; CC(C)c1cnc(Br)cc1Oc1cnc(N)nc1NCC(O)CO]

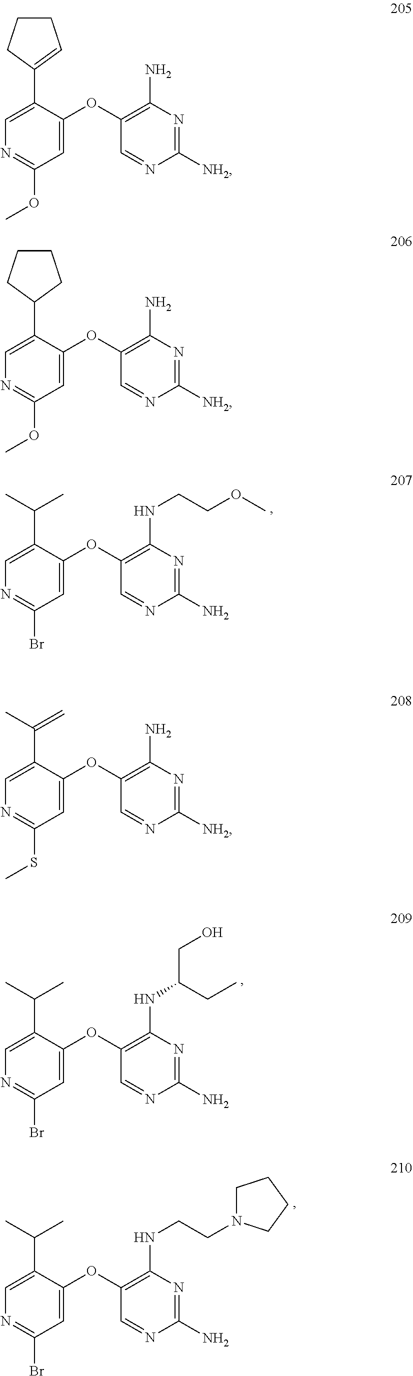

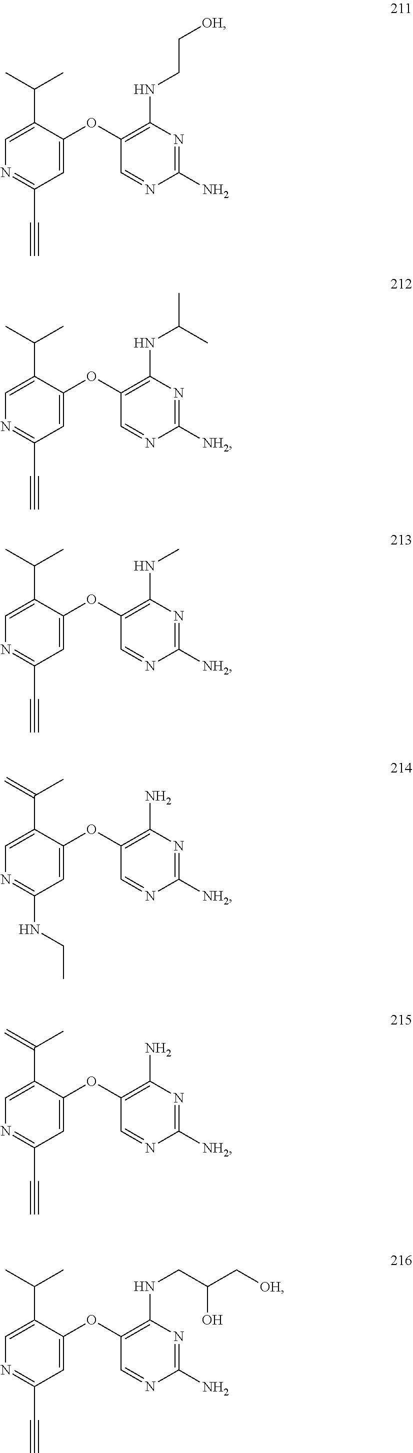 [SMILES: C#Cc1cc(Oc2cnc(N)nc2NCCO)c(C(C)C)cn1 ; C#Cc1cc(Oc2cnc(N)nc2NC(C)C)c(C(C)C)cn1 ; C#Cc1cc(Oc2cnc(N)nc2NC)c(C(C)C)cn1 ; C=C(C)c1cnc(NCC)cc1Oc1cnc(N)nc1N ; C#Cc1cc(Oc2cnc(N)nc2N)c(C(=C)C)cn1 ; C#Cc1cc(Oc2cnc(N)nc2NCC(O)CO)c(C(C)C)cn1]

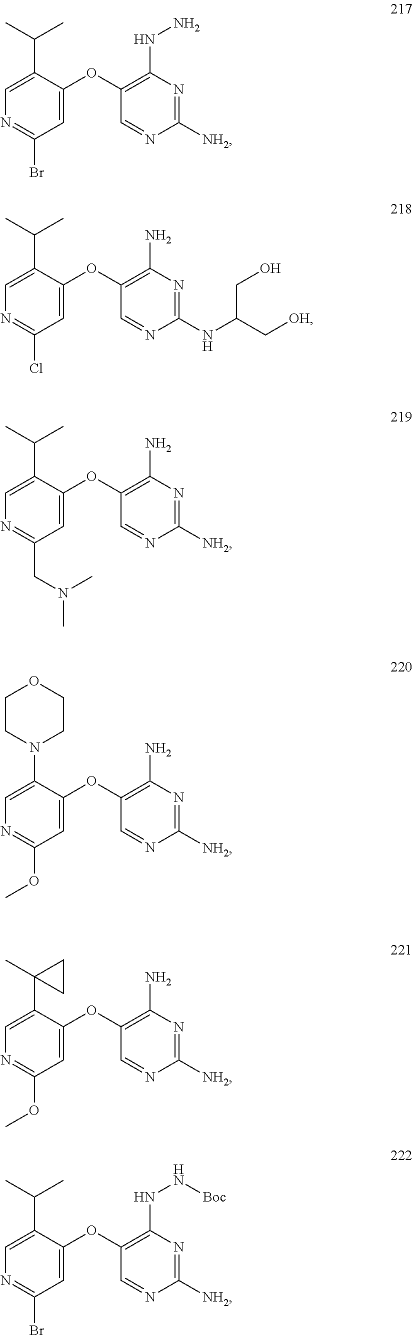 [SMILES: CC(C)c1cnc(Br)cc1Oc1cnc(N)nc1NN ; CC(C)c1cnc(Cl)cc1Oc1cnc(NC(CO)CO)nc1N ; CC(C)c1cnc(CN(C)C)cc1Oc1cnc(N)nc1N ; COc1cc(Oc2cnc(N)nc2N)c(N2CCOCC2)cn1 ; COc1cc(Oc2cnc(N)nc2N)c(C2(C)CC2)cn1 ; CC(C)c1cnc(Br)cc1Oc1cnc(N)nc1NNC(=O)OC(C)(C)C]

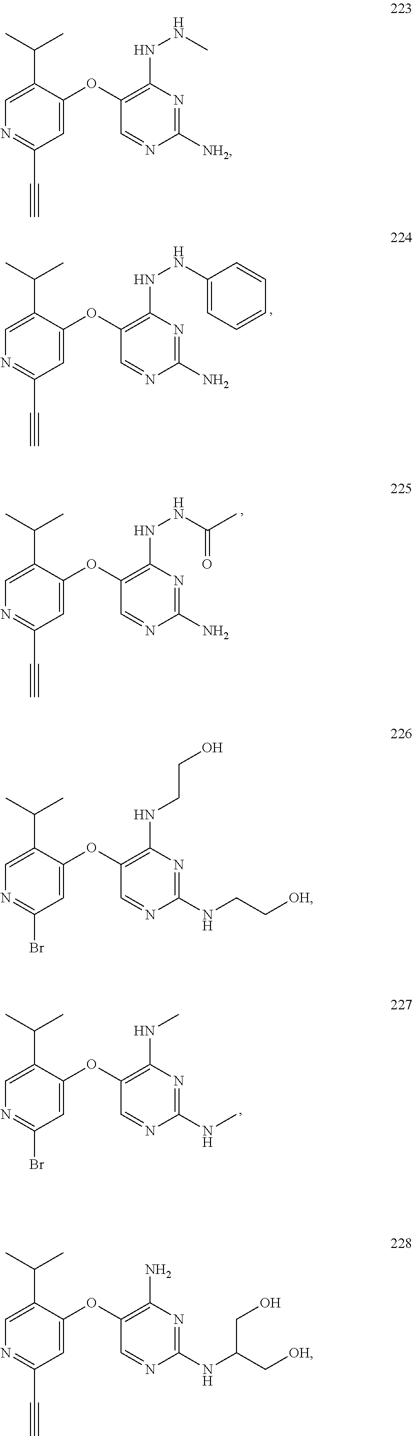 [SMILES: C#Cc1cc(Oc2cnc(N)nc2NNC)c(C(C)C)cn1 ; C#Cc1cc(Oc2cnc(N)nc2NNc2ccccc2)c(C(C)C)cn1 ; C#Cc1cc(Oc2cnc(N)nc2NNC(C)=O)c(C(C)C)cn1 ; CC(C)c1cnc(Br)cc1Oc1cnc(NCCO)nc1NCCO ; CNc1ncc(Oc2cc(Br)ncc2C(C)C)c(NC)n1 ; C#Cc1cc(Oc2cnc(NC(CO)CO)nc2N)c(C(C)C)cn1]

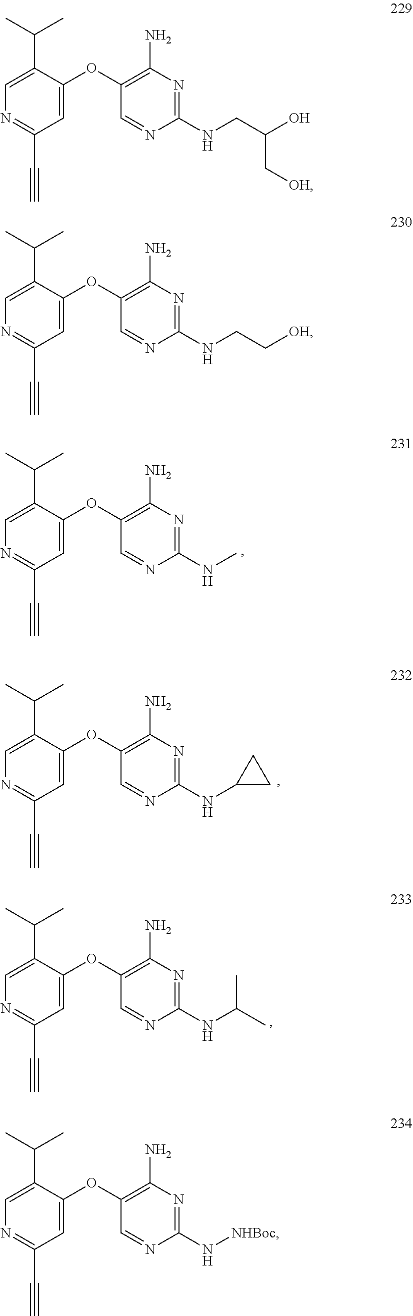

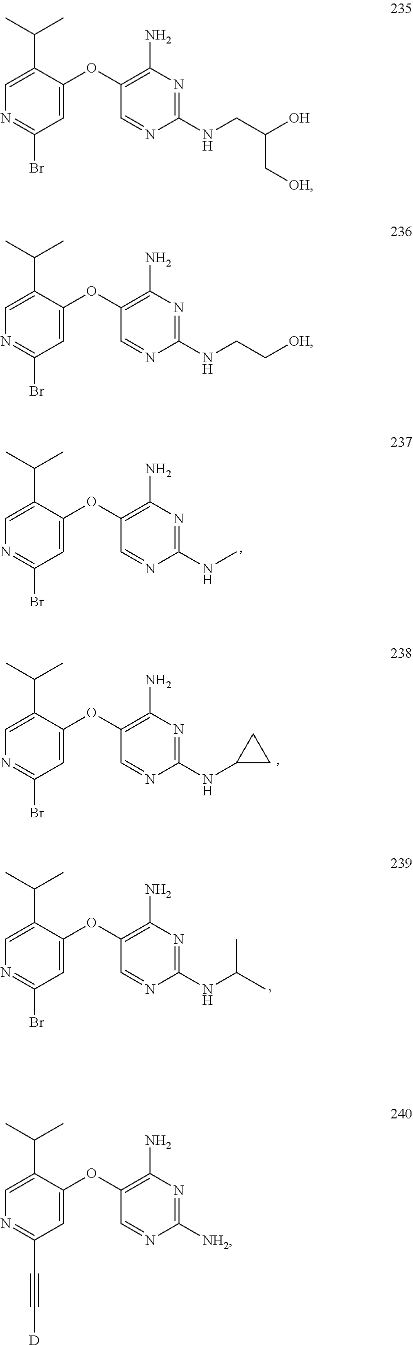 [SMILES: CC(C)c1cnc(Br)cc1Oc1cnc(NCC(O)CO)nc1N ; CC(C)c1cnc(Br)cc1Oc1cnc(NCCO)nc1N ; CNc1ncc(Oc2cc(Br)ncc2C(C)C)c(N)n1 ; CC(C)c1cnc(Br)cc1Oc1cnc(NC2CC2)nc1N ; CC(C)Nc1ncc(Oc2cc(Br)ncc2C(C)C)c(N)n1 ; [2H]C#Cc1cc(Oc2cnc(N)nc2N)c(C(C)C)cn1]

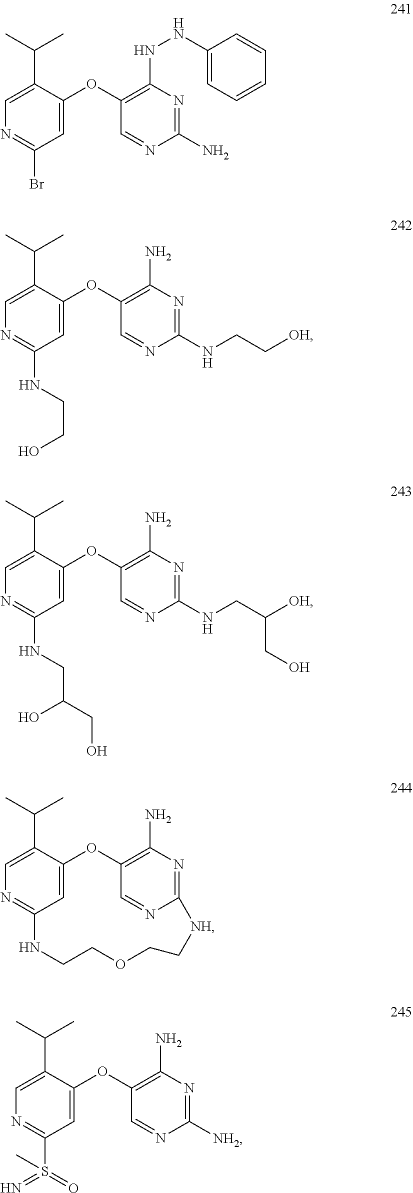 [SMILES: CC(C)c1cnc(Br)cc1Oc1cnc(N)nc1NNc1ccccc1 ; CC(C)c1cnc(NCCO)cc1Oc1cnc(NCCO)nc1N ; CC(C)c1cnc(NCC(O)CO)cc1Oc1cnc(NCC(O)CO)nc1N ; CC(C)c1cnc2cc1Oc1cnc(nc1N)NCCOCCN2 ; CC(C)c1cnc(S(C)(=N)=O)cc1Oc1cnc(N)nc1N]

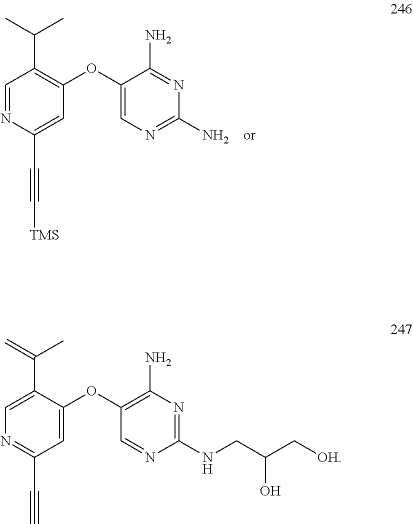 [SMILES: CC(C)c1cnc(C#C[Si](C)(C)C)cc1Oc1cnc(N)nc1N ; C#Cc1cc(Oc2cnc(NCC(O)CO)nc2N)c(C(=C)C)cn1]

15. The method according to claim 12, wherein the compound of Formula (I) or a pharmaceutically acceptable salt, ester, stereoisomer, polymorph, solvate, N-oxide, isotopically labeled compound, metabolite or prodmg thereof is administered in an amount of about 0.005 mg/day to about 5000 mg/day, e.g., in an amount of about 0.005, 0.05, 0.5, 5, 10, 20, 30, 40, 50, 100, 150, 200, 250, 300, 350, 400, 450, 500, 550, 600, 650, 700, 750, 800, 850, 900, 950, 1000, 1500, 2000, 2500, 3000, 3500, 4000, 4500 or 5000 mg/day.

16. The method according to claim 12, wherein the compound of Formula (I) or a pharmaceutically acceptable salt, ester, stereoisomer, polymorph, solvate, N-oxide, isotopically labeled compound, metabolite or prodrug thereof is administered in an amount of about 1 ng/kg to about 200 mg/kg, about 1 μg/kg to about 100 mg/kg or about 1 mg/kg to about 50 mg/kg per day, e.g., is administered in an amount of about 1 μg/kg, about 10 μg/kg, about 25 μg/kg, about 50 μg/kg, about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 525 μg/kg, about 550 μg/kg, about 575 μg/kg, about 600 μg/kg, about 625 μg/kg, about 650 μg/kg, about 675 μg/kg, about 700 μg/kg, about 725 μg/kg, about 750 μg/kg, about 775 μg/kg, about 800 μg/kg, about 825 μg/kg, about 850 μg/kg, about 875 μg/kg, about 900 μg/kg, about 925 μg/kg, about 950 μg/kg, about 975 μg/kg, about 1 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg, about 90 mg/kg, about 100 mg/kg, about 125 mg/kg, about 150 mg/kg, about 175 mg/kg, about 200 mg/kg body weight per unit dose.

17. The method according to claim 12, wherein the daily dose of the compound of Formula (I) or a pharmaceutically acceptable salt, ester, stereoisomer, polymorph, solvate, N-oxide, isotopically labeled compound, metabolite or prodmg thereof is administered at one time or is administered in two, three or four doses.

18. The method according to claim 12, wherein the compound of Formula (I) or a pharmaceutically acceptable salt, ester, stereoisomer, polymorph, solvate, N-oxide, isotopically labeled compound, metabolite or prodrug thereof is administered continuously for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days, at least 50 days, at least half a year, at least 1 year, at least 2 years, at least 3 years, at least 4 years, or at least 5 years.

19. The method according to claim 12, wherein the compound of Formula (I) or a pharmaceutically acceptable salt, ester, stereoisomer, polymorph, solvate, N-oxide, isotopically labeled compound, metabolite or prodrug thereof is administered for one or more (e.g., 1, 2, 3, 4, 5, 6, 7, 8, 9 or 10) courses of treatment, wherein each course of treatment lasts for at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 30 days, at least 35 days, at least 40 days, at least 45 days or at least 50 days; and the interval between every two courses of treatment is 0, 1, 2, 3, 4, 5, 6, 7, 8, 9, 10 days, two weeks, three weeks, or four weeks.

20. The method according to claim 12, wherein the compound of Formula (I) or a pharmaceutically acceptable salt, ester, stereoisomer, polymorph, solvate, N-oxide, isotopically labeled compound, metabolite or prodrug thereof is administered through injection (e.g., intravenous, intraarterial, subcutaneous, intraperitoneal, intramuscular injection, including dripping), or transdermal administration, or is administered via oral, buccal, nasal, transmucosal, or topical route, as an ophthalmic formulation, or via inhalation.

21. The method according to claim 12, wherein the compound of Formula (I) or a pharmaceutically acceptable salt, ester, stereoisomer, polymorph, solvate, N-oxide, isotopically labeled compound, metabolite or prodrug thereof is administered in a dosage form selected from the group consisting of tablet, capsule, lozenge, hard candy, powder, spray, emulsion, cream, salve, suppository, gel, paste, lotion, injection, nanoformulation, patch, aqueous suspension, solution, elixir, and syrup.

22. The method according to claim 12, wherein the cough is selected from the group consisting of acute cough, sub-acute cough, chronic cough, treatment-resistant cough, treatment-resistant chronic cough, idiopathic chronic cough, post-viral cough, iatrogenic cough, cough associated with post-nasal drip, cough associated with cold, upper respiratory infection, asthma, lung cancer and/or chronic obstructive pulmonary disease (COPD), cough associated with interstitial disease, cough associated with pulmonary fibrosis, cough associated with gastroesophageal reflux disease (GERD), cough associated with smoking or a form of bronchitis, and neuronal hypersensitivity underlying acute, sub-acute or chronic cough; preferably, the cough is selected from the group consisting of chronic cough, treatment-resistant cough, treatment-resistant chronic cough, and cough associated with pulmonary fibrosis.
